(19) United States
(12) Patent Application Publication
(10) Pub. No.: US 2026/0349337 A1
(43) Pub. Date: Nov. 5, 2026

(54) ACTINIC RAY-SENSITIVE OR RADIATION-SENSITIVE RESIN COMPOSITION, RESIST FILM, PATTERN FORMING METHOD, AND METHOD FOR PRODUCING ELECTRONIC DEVICE

(71) Applicant: FUJIFILM Corporation, Tokyo (JP)

(72) Inventors: Ryosuke Kato, Shizuoka (JP); Hokuto Yamazaki, Shizuoka (JP); Masafumi Kojima, Shizuoka (JP); Akiyoshi Goto, Shizuoka (JP)

(21) Appl. No.: 19/559,155

(22) Filed: Mar. 6, 2026

Related U.S. Application Data
(63) Continuation of application No. PCT/JP2024/030066, filed on Aug. 23, 2024.

(30) Foreign Application Priority Data
Sep. 23, 2023 (JP) 2023-148780

(51) Int. Cl. G03F 7/004; G03F 7/038; G03F 7/039

(52) U.S. Cl. CPC G03F 7/0045; G03F 7/038; G03F 7/039

(57) ABSTRACT

Provided are an actinic ray-sensitive or radiation-sensitive resin composition that includes a resin and a compound having a specific structure and having an iodine atom, a resist film formed using the actinic ray-sensitive or radiation-sensitive resin composition, and a pattern forming method and a method for producing an electronic device that use the actinic ray-sensitive or radiation-sensitive resin composition.

### CROSS REFERENCE TO RELATED APPLICATION

This is a continuation of International Application No. PCT/JP2024/030066 filed on Aug. 23, 2024, and claims priority from Japanese Patent Application No. 2023-148780 filed on Sep. 13, 2023, the entire disclosure of which is incorporated herein by reference.

### BACKGROUND OF THE INVENTION

### 1. Field of the Invention

The present invention relates to an actinic ray-sensitive or radiation-sensitive resin composition, a resist film, a pattern forming method, and a method for producing an electronic device. More specifically, the present invention relates to an actinic ray-sensitive or radiation-sensitive resin composition that can be suitably used in ultra-microlithography processes applicable to, for example, processes for producing ultra-LSIs (Large Scale Integrations) and high-capacity microchips, processes for preparing nanoimprint molds, and processes for producing high-density information recording media, and other photofabrication processes and relates to a resist film, a pattern forming method, and a method for producing an electronic device.

### 2. Description of the Related Art

In processes for producing semiconductor devices such as ICs (Integrated Circuits) or LSIs (Large Scale Integrations), fine processing by lithography using a resist composition has been conventionally performed. In recent years, along with the high integration of integrated circuits, formation of ultrafine patterns in the submicron range or quarter-micron range has been required. Accordingly, exposure wavelengths also tend to be shortened from the g-line to the i-line and further to KrF excimer laser light, and exposure apparatuses using ArF excimer laser having a wavelength of 193 nm as light sources have currently been developed. In addition, as a technique for further increasing the resolving power, a so-called immersion method has been developed in which a gap between a projection lens and a sample is filled with a liquid having a high refractive index (hereinafter, also referred to as an “immersion liquid”).

At present, lithography using electron beams (EB), X-rays, extreme ultraviolet rays (EUV), or the like instead of excimer laser light is also being developed. Accordingly, resist compositions that are effectively sensitive to various types of actinic rays or radiation have been developed.

JP2011-95700A discloses an actinic ray-sensitive or radiation-sensitive resin composition including a compound that generates an acid having a specific structure upon irradiation with actinic rays or radiation.

### SUMMARY OF THE INVENTION

In recent years, performance required for resist compositions has become increasingly higher. In particular, it is required to improve the sensitivity and pattern shape (rectangularity of a pattern) when a fine pattern (for example, a 1:1 line-and-space pattern with a line width of 30 nm) is formed.

An object of the present invention is to provide an actinic ray-sensitive or radiation-sensitive resin composition that has high sensitivity and that provides a pattern having a good shape.

Another object of the present invention is to provide a resist film formed using the actinic ray-sensitive or radiation-sensitive resin composition, and a pattern forming method and a method for producing an electronic device that use the actinic ray-sensitive or radiation-sensitive resin composition.

The inventors of the present invention have found that the above objects can be achieved by the following configurations.

[1]

An actinic ray-sensitive or radiation-sensitive resin composition including a compound (N) represented by a formula (a-1) below and a resin.

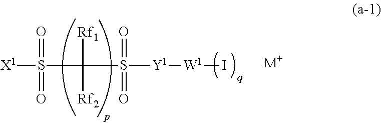

In the formula (a-1),
X¹ represents —O⁻ or —N⁻—SO₂—Rf₃.
Rf₁, Rf₂, and Rf₃ each independently represent a fluorine atom or an alkyl group having at least one fluorine atom. When a plurality of Rf₁'s and a plurality of Rf₂'s are present, the plurality of Rf₁'s may be the same or different and the plurality of Rf₂'s may be the same or different.
Y¹ represents a single bond, —O—, or —S—.
W¹ represents an organic group.
p represents an integer of 1 or more.
q represents an integer of 1 or more.
M⁺ represents a cation.
   [2]

The actinic ray-sensitive or radiation-sensitive resin composition according to [1], wherein the compound (N) is represented by a formula (a-2) below.

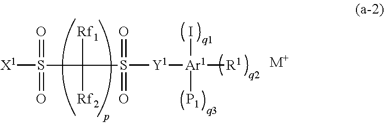

In the formula (a-2),
X¹ represents —O⁻ or —N⁻—SO₂—Rf₃.
Rf₁, Rf₂, and Rf₃ each independently represent a fluorine atom or an alkyl group having at least one fluorine atom. When a plurality of Rf₁'s and a plurality of Rf₂'s are present, the plurality of Rf₁'s may be the same or different and the plurality of Rf₂'s may be the same or different.
Y¹ represents a single bond, —O—, or —S—.
Ar¹ represents an aromatic group.
R¹ represents an organic group having an iodine atom.
p represents an integer of 1 or more.
q1 and q2 each independently represent an integer of 0 or more, provided that at least one of q1 or q2 represents an integer of 1 or more.
P¹ represents an organic group having no iodine atom.
q3 represents an integer of 0 or more.
M⁺ represents a cation.
   [3]

The actinic ray-sensitive or radiation-sensitive resin composition according to [1] or [2], wherein X¹ in the formula (a-1) or the formula (a-2) represents —O⁻.

[4]

The actinic ray-sensitive or radiation-sensitive resin composition according to [2], wherein q1 in the formula (a-2) represents an integer of 1 or more.

[5]

The actinic ray-sensitive or radiation-sensitive resin composition according to [2] or [4], wherein q2 in the formula (a-2) represents an integer of 1 or more.

[6]

The actinic ray-sensitive or radiation-sensitive resin composition according to any one of [1] to [5], wherein p in the formula (a-1) or the formula (a-2) represents an integer of 1 to 3.

[7]

The actinic ray-sensitive or radiation-sensitive resin composition according to [2], [4], or [5], wherein at least one R¹ in the formula (a-2) represents —CO—O—R², —O—CO—R², —O—CO—O—R², —SO₂—R², or —SO₃—R², where R² represents an organic group having an iodine atom.

[8]

The actinic ray-sensitive or radiation-sensitive resin composition according to [2], [4], [5], or [7], wherein q3 in the formula (a-2) represents an integer of 1 or more, and at least one P¹ represents —CO—O—P², —O—CO—P², —O—CO—O—P², —SO₂—P², or —SO₃—P², where P² represents an organic group having no iodine atom.

[9]

The actinic ray-sensitive or radiation-sensitive resin composition according to any one of [1] to [8], wherein M⁺ in the formula (a-1) or the formula (a-2) represents a sulfonium cation or an iodonium cation.

[10]

The actinic ray-sensitive or radiation-sensitive resin composition according to [1], [3], [6], or [9], wherein q in the formula (a-1) or the formula (a-2) represents an integer of 1 to 20.

[11]

The actinic ray-sensitive or radiation-sensitive resin composition according to [2], [4], [5], [7], or [8], wherein R¹ in the formula (a-2) has no aromatic ring.

[12]

A resist film formed using the actinic ray-sensitive or radiation-sensitive resin composition according to any one of [1] to [11].

[13]

A pattern forming method including forming a resist film on a substrate using the actinic ray-sensitive or radiation-sensitive resin composition according to any one of [1] to [11]; exposing the resist film; and developing the exposed resist film using a developer.

[14]

A method for producing an electronic device, the method including the pattern forming method according to [13].

The present invention can provide an actinic ray-sensitive or radiation-sensitive resin composition that has high sensitivity and that provides a pattern having a good shape.

Furthermore, the present invention can provide a resist film formed using the actinic ray-sensitive or radiation-sensitive resin composition, and a pattern forming method and a method for producing an electronic device that use the actinic ray-sensitive or radiation-sensitive resin composition.

### DESCRIPTION OF THE PREFERRED EMBODIMENTS

The present invention will be described in detail below.

Constituent features may be described below on the basis of representative embodiments of the present invention; however, the present invention is not limited to such embodiments.

In the present specification, “actinic rays” or “radiation” means, for example, a bright line spectrum of a mercury lamp, far ultraviolet rays typified by excimer laser, extreme ultraviolet rays (EUV: Extreme Ultraviolet), X-rays, soft X rays, and an electron beam (EB: Electron Beam).

In the present specification, “light” means actinic rays or radiation.

In the present specification, unless otherwise specified, the “exposure” includes not only exposure to a bright line spectrum of a mercury lamp, far ultraviolet rays typified by excimer laser, extreme ultraviolet rays, X-rays, or the like, but also patterning using an electron beam or a particle beam such as an ion beam.

In the present specification, a range of numerical values expressed with “to” means a range that includes a numerical value before “to” as a lower limit value and a numerical value after “to” as an upper limit value.

In the present specification, (meth)acrylate represents at least one of acrylate or methacrylate. (Meth)acrylic acid represents at least one of acrylic acid or methacrylic acid.

In the present specification, the weight-average molecular weight (Mw), the number-average molecular weight (Mn), and the dispersity (also referred to as molecular weight distribution) (Mw/Mn) of a resin are defined as values in terms of polystyrene determined, using a GPC (Gel Permeation Chromatography) apparatus (HLC-8120GPC manufactured by Tosoh Corporation), by GPC measurement (solvent: tetrahydrofuran, amount of flow (amount of sample injected): 10 μL, column: TSK gel Multipore HXL-M manufactured by Tosoh Corporation, column temperature: 40 °C, flow rate: 1.0 mL/min, detector: differential refractive index detector (Refractive Index Detector)).

Regarding notations of groups (atomic groups) in the present specification, a notation without specifying whether substituted or unsubstituted also encompasses a group including a substituent as well as a group having no substituent without departing from the spirit and scope of the present invention. For example, an “alkyl group” encompasses not only an alkyl group having no substituent (an unsubstituted alkyl group) but also an alkyl group having a substituent (a substituted alkyl group). In the present specification, an “organic group” refers to a group including at least one carbon atom.

As the substituent, a monovalent substituent is preferable unless otherwise specified. Examples of the substituent include monovalent non-metallic atomic groups excluding a hydrogen atom, and, for example, the substituent can be selected from the group consisting of the following substituents T.

### (Substituents T)

Examples of the substituents T include halogen atoms such as a fluorine atom, a chlorine atom, a bromine atom, and an iodine atom; alkoxy groups such as a methoxy group, an ethoxy group, and a tert-butoxy group; cycloalkyloxy groups; aryloxy groups such as a phenoxy group and a p-tolyloxy group; alkoxycarbonyl groups such as a methoxycarbonyl group and a butoxycarbonyl group; cycloalkyloxycarbonyl groups; aryloxycarbonyl groups such as a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; a sulfanyl group; alkylsulfanyl groups such as a methylsulfanyl group and a tert-butylsulfanyl group; arylsulfanyl groups such as a phenylsulfanyl group and a p-tolylsulfanyl group; alkyl groups; alkenyl groups; cycloalkyl groups; aryl groups; aromatic heterocyclic groups; a hydroxy group; a carboxyl group; a formyl group; a sulfo group; a cyano group; alkylaminocarbonyl groups; arylaminocarbonyl groups; a sulfonamide group; silyl groups; an amino group; and a carbamoyl group. When these substituents can further have one or more substituents, groups having, as the additional substituents, one or more substituents selected from the group consisting of the aforementioned substituents (for example, monoalkylamino groups, dialkylamino groups, arylamino groups, a trifluoromethyl group, and the like) are also included in the examples of the substituents T.

The bonding direction of a divalent group expressed in the present specification is not limited unless otherwise specified. For example, in a compound represented by a formula “X—Y—Z” where Y is —COO—, Y may be —CO—O— or —O—CO—. The compound may be “X—CO—O—Z” or “X—O—CO—Z”.

In the present specification, the acid dissociation constant (pKa) represents pKa in an aqueous solution, and specifically, is a value determined by calculation using the following software package 1 on the basis of the Hammett substituent constant and a database of values in publicly known documents. All the values of pKa described in the present specification are values determined by calculation using this software package.

Software package 1: Advanced Chemistry Development (ACD/Labs) Software V8.14 for Solaris (1994-2007 ACD/Labs)

Alternatively, the pKa can be determined by molecular orbital calculation methods. A specific method thereof may be a method of calculating the pKa by calculating H⁺ dissociation free energy in an aqueous solution on the basis of a thermodynamic cycle. With regard to the method of calculating the H⁺ dissociation free energy, the calculation can be performed by, for example, DFT (density functional theory); however, various other methods have been reported in documents etc., and the method is not limited to this. There are a plurality of pieces of software capable of performing DFT, and an example thereof is Gaussian 16.

In the present specification, as described above, the pKa refers to a value determined by calculation using the software package 1 on the basis of the Hammett substituent constant and the database of values in publicly known documents; however, when the pKa cannot be calculated by this method, a value determined using Gaussian16 based on DFT (density functional theory) is employed.

In the present specification, the pKa refers to “pKa in an aqueous solution” as described above; however when the pKa in an aqueous solution cannot be calculated, “pKa in a dimethyl sulfoxide (DMSO) solution” is employed.

In the present specification, the “solid contents” mean components that form a film (preferably, a resist film) formed using an actinic ray-sensitive or radiation-sensitive resin composition, and do not include a solvent. In addition, even if a component that forms a film (preferably, a resist film) formed using an actinic ray-sensitive or radiation-sensitive resin composition is in a liquid state, the component is considered as a solid content.

### <Actinic Ray-Sensitive or Radiation-Sensitive Resin Composition>

An actinic ray-sensitive or radiation-sensitive resin composition of the present invention (also referred to as a “composition of the present invention”) is an actinic ray-sensitive or radiation-sensitive resin composition including a compound (N) represented by a formula (a-1) below and a resin.

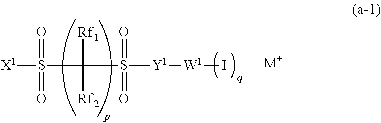

In the formula (a-1),
X¹ represents —O⁻ or —N⁻—SO₂—Rf₃.
Rf₁, Rf₂, and Rf₃ each independently represent a fluorine atom or an alkyl group having at least one fluorine atom. When a plurality of Rf₁'s and a plurality of Rf₂'s are present, the plurality of Rf₁'s may be the same or different and the plurality of Rf₂'s may be the same or different.
Y¹ represents a single bond, —O—, or —S—.
W¹ represents an organic group.
p represents an integer of 1 or more.
q represents an integer of 1 or more.
M⁺ represents a cation.

The mechanism by which the composition of the present invention has high sensitivity and provides a pattern having a good shape is not clear, but the inventors of the present invention presume as follows. However, the present invention is not limited at all by the following presumed mechanism.

The compound (N) included in the composition of the present invention can generate a strong acid and has an iodine atom, which has a strong interaction with an acid (has a strong proton acceptor property). Thus, it is considered that the combination of the strong acid and the iodine atom suppresses the diffusion of the acid and improves the pattern shape (makes the pattern shape more rectangular). In addition, it is considered that since the compound (N) has an iodine atom, the absorption efficiency of exposure light (for example, EUV or the like) increases, and the sensitivity is improved.

The composition of the present invention is typically a resist composition, and may be either a positive resist composition or a negative resist composition. The composition of the present invention may be a resist composition for alkali development or a resist composition for organic solvent development.

The composition of the present invention may be a chemical amplification resist composition or a non-chemical amplification resist composition. The composition of the present invention is preferably a chemical amplification resist composition.

The composition of the present invention can be used to form an actinic ray-sensitive or radiation-sensitive film. The actinic ray-sensitive or radiation-sensitive film formed using the composition of the present invention is typically a resist film.

First, various components of the composition of the present invention will be described in detail below.

### [Compound (N)]

The composition of the present invention includes a compound (N) represented by the formula (a-1) (also simply referred to as a “compound (N)”).

The compound (N) is preferably a compound that generates an acid upon irradiation with actinic rays or radiation (photoacid generator).

When the compound (N) is a photoacid generator, the pKa of the acid generated from the compound (N) upon irradiation with actinic rays or radiation (generated acid) is preferably −2 or less, more preferably −3 or less, still more preferably −3.4 or less. The pKa of the generated acid is preferably −12 or more, more preferably −11 or more, still more preferably −10 or more. In the formula (a-1), X¹ represents —O⁻ or —N⁻—SO₂—Rf₃.

Rf₃ represents a fluorine atom or an alkyl group having at least one fluorine atom.

The alkyl group having at least one fluorine atom (fluoroalkyl group) represented by Rf₃ may be linear or branched. The number of carbon atoms of the fluoroalkyl group is not particularly limited. For example, a fluoroalkyl group having 1 to 20 carbon atoms is preferred, a fluoroalkyl group having 1 to 15 carbon atoms is more preferred, a fluoroalkyl group having 1 to 10 carbon atoms is still more preferred, and a fluoroalkyl group having 1 to 6 carbon atoms is particularly preferred. The fluoroalkyl group represented by Rf₃ is preferably a perfluoroalkyl group. The fluoroalkyl group represented by Rf₃ may have a substituent other than a fluorine atom. Rf₃ preferably represents a fluoroalkyl group and particularly preferably represents a trifluoromethyl group.

X¹ preferably represents —O⁻.

In the formula (a-1), Rf₁ and Rf₂ each independently represent a fluorine atom or an alkyl group having at least one fluorine atom. When a plurality of Rf₁'s and a plurality of Rf₂'s are present, the plurality of Rf₁'s may be the same or different and the plurality of Rf₂'s may be the same or different.

The description, specific examples, and preferred ranges of the alkyl group having at least one fluorine atom (fluoroalkyl group) represented by Rf₁ and Rf₂ are respectively the same as those of the fluoroalkyl group represented by Rf₃.

Rf₁ and Rf₂ each preferably represent a fluorine atom.

In the formula (a-1), Y¹ represents a single bond, —O—, or —S—.

Y¹ preferably represents a single bond or —O—, and more preferably represents —O—.

In the formula (a-1), W¹ represents an organic group.

The organic group represented by W¹ is not particularly limited. The number of carbon atoms of the organic group represented by W¹ is preferably 1 to 100, more preferably 3 to 70, still more preferably 5 to 50.

The organic group represented by W¹ is preferably a group having at least one aromatic ring. The aromatic ring may be an aromatic hydrocarbon ring or an aromatic heterocycle, but is preferably an aromatic hydrocarbon ring. The number of carbon atoms of the aromatic hydrocarbon ring is preferably 6 to 20, more preferably 6 to 15, still more preferably 6 to 10. The aromatic heterocycle preferably includes at least one heteroatom selected from the group consisting of a nitrogen atom, an oxygen atom, and a sulfur atom as a ring member. The number of ring member atoms of the aromatic heterocycle is not particularly limited, but is preferably 3 to 30, more preferably 4 to 20. The number of carbon atoms of the aromatic heterocycle is not particularly limited, but is preferably 2 to 20, more preferably 3 to 15.

The organic group represented by W¹ may have a substituent other than an iodine atom.

When the organic group represented by W¹ is a group having an aromatic ring, an iodine atom may be bonded to the aromatic ring, or an iodine atom may be bonded to a substituent bonded to the aromatic ring.

In the formula (a-1), p represents an integer of 1 or more, preferably represents an integer of 1 to 10, more preferably represents an integer of 1 to 6, and still more preferably represents an integer of 1 to 3.

In the formula (a-1), q represents an integer of 1 or more, preferably represents an integer of 1 to 20, more preferably represents an integer of 1 to 10, and still more preferably represents an integer of 1 to 6.

In the formula (a-1), M⁺ represents a cation, preferably represents an organic cation, and more preferably represents a sulfonium cation or an iodonium cation.

The cation represented by M⁺ is not particularly limited. The valence of the cation may be mono-, di-, or higher valent. The cation is preferably a cation represented by a formula (ZaI) below (hereinafter, also referred to as a “cation (ZaI)”) or a cation represented by a formula (ZaII) below (hereinafter, also referred to as a “cation (ZaII)”).

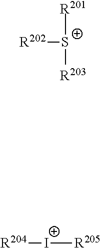

In the formula (ZaI), R²⁰¹, R²⁰², and R²⁰³ each independently represent an organic group.

The number of carbon atoms of each of the organic groups serving as R²⁰¹, R²⁰², and R²⁰³ is preferably 1 to 30, more preferably 1 to 20. Two of R²⁰¹ to R²⁰³ may be bonded together to form a ring structure, and the ring may include an oxygen atom, a sulfur atom, an ester group, an amide group, or a carbonyl group. Examples of the group formed by bonding two of R²⁰¹ to R²⁰³ together include alkylene groups (such as a butylene group and a pentylene group) and —CH₂—CH₂—O—CH₂—CH₂—.

Preferred embodiments of the organic cation in the formula (ZaI) include a cation (ZaI-1), a cation (ZaI-2), a cation (ZaI-3b), and a cation (ZaI-4b) described below. First, the cation (ZaI-1) will be described.

The cation (ZaI-1) is an arylsulfonium cation in which at least one of R²⁰¹ to R²⁰³ in the formula (ZaI) is an aryl group.

In the arylsulfonium cation, all of R²⁰¹ to R²⁰³ may be aryl groups, or some of R²⁰¹ to R²⁰³ may be an aryl group and the remainder may be an alkyl group or a cycloalkyl group.

One of R²⁰¹ to R²⁰³ may be an aryl group and the other two of R²⁰¹ to R²⁰³ may be bonded together to form a ring structure, and the ring may include an oxygen atom, a sulfur atom, an ester group, an amide group, or a carbonyl group. Examples of the group formed by bonding two of R²⁰¹ to R²⁰³ together include alkylene groups in which one or more methylene groups may be substituted with an oxygen atom, a sulfur atom, an ester group, an amide group, and/or a carbonyl group (such as a butylene group, a pentylene group, and —CH₂—CH₂—O—CH₂—CH₂—).

Examples of the arylsulfonium cation include triarylsulfonium cations, diarylalkylsulfonium cations, aryldialkylsulfonium cations, diarylcycloalkylsulfonium cations, and aryldicycloalkylsulfonium cations.

The aryl group included in the arylsulfonium cation is preferably a phenyl group or a naphthyl group, more preferably a phenyl group. The aryl group may be an aryl group having a heterocyclic structure having, for example, an oxygen atom, a nitrogen atom, or a sulfur atom. Examples of the heterocyclic structure include a pyrrole residue, a furan residue, a thiophene residue, an indole residue, a benzofuran residue, and a benzothiophene residue. When the arylsulfonium cation has two or more aryl groups, the two or more aryl groups may be the same or different.

The alkyl group or the cycloalkyl group that the arylsulfonium cation optionally has is preferably a linear alkyl group having 1 to 15 carbon atoms, a branched alkyl group having 3 to 15 carbon atoms, or a cycloalkyl group having 3 to 15 carbon atoms, more preferably a methyl group, an ethyl group, a propyl group, a n-butyl group, a sec-butyl group, a t-butyl group, a cyclopropyl group, a cyclobutyl group, or a cyclohexyl group.

Substituents that the aryl group, the alkyl group, and the cycloalkyl group in R²⁰¹ to R²⁰³ may have are each preferably an alkyl group (having, for example, 1 to 15 carbon atoms), a cycloalkyl group (having, for example, 3 to 15 carbon atoms), an aryl group (having, for example, 6 to 14 carbon atoms), an alkoxy group (having, for example, 1 to 15 carbon atoms), a cycloalkylalkoxy group (having, for example, 1 to 15 carbon atoms), a halogen atom other than a fluorine atom (for example, a chlorine atom, a bromine atom, or an iodine atom), a hydroxy group, a carboxyl group, an ester group, a sulfinyl group, a sulfonyl group, an alkylthio group, or a phenylthio group.

The substituent may further have a substituent, if possible.

It is also preferable that any combination of the above substituents form an acid-decomposable group.

Next, the cation (ZaI-2) will be described.

The cation (ZaI-2) is a cation in which R²⁰¹ to R²⁰³ in the formula (ZaI) each independently represent an organic group having no aromatic ring. The aromatic ring also encompasses an aromatic ring including a heteroatom.

The number of carbon atoms of each of the organic groups having no aromatic ring and serving as R²⁰¹ to R²⁰³ is preferably 1 to 30, more preferably 1 to 20.

R²⁰¹ to R²⁰³ are each independently preferably an alkyl group, a cycloalkyl group, an allyl group, or a vinyl group, more preferably a linear or branched 2-oxoalkyl group, a 2-oxocycloalkyl group, or an alkoxycarbonylmethyl group, still more preferably a linear or branched 2-oxoalkyl group.

Examples of the alkyl group and the cycloalkyl group in R²⁰¹ to R²⁰³ include linear alkyl groups having 1 to 10 carbon atoms and branched alkyl groups having 3 to 10 carbon atoms (such as a methyl group, an ethyl group, a propyl group, a butyl group, and a pentyl group), and cycloalkyl groups having 3 to 10 carbon atoms (such as a cyclopentyl group, a cyclohexyl group, and a norbornyl group).

R²⁰¹ to R²⁰³ may be further substituted with a halogen atom other than a fluorine atom, an alkoxy group (having, for example, 1 to 5 carbon atoms), a hydroxy group, a cyano group, or a nitro group.

It is also preferable that the substituents in R²⁰¹ to R²⁰³ each independently form an acid-decomposable group by any combination of substituents.

Next, the cation (ZaI-3b) will be described.

The cation (ZaI-3b) is a cation represented by a formula (ZaI-3b) below.

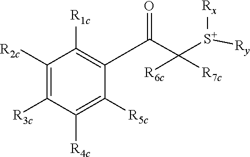

In the formula (ZaI-3b), R_{1c} to R_{5c} each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom other than a fluorine atom, a hydroxy group, a nitro group, an alkylthio group, or an arylthio group.

R_{6c} and R_{7c} each independently represent a hydrogen atom, an alkyl group (such as a t-butyl group), a cycloalkyl group, a halogen atom other than a fluorine atom, a cyano group, or an aryl group.

Rₓ and R_{y} each independently represent an alkyl group, a cycloalkyl group, a 2-oxoalkyl group, a 2-oxocycloalkyl group, an alkoxycarbonylalkyl group, an allyl group, or a vinyl group.

It is also preferable that substituents in R_{1c} to R_{7c} and Rₓ and R_{y} each independently form an acid-decomposable group by any combination of substituents.

Any two or more of R_{1c} to R_{5c}, R_{5c} and R_{6c}, R_{6c} and R_{7c}, R_{5c} and Rₓ, and Rₓ and R_{y} may be individually bonded together to form rings, and these rings may each independently include an oxygen atom, a sulfur atom, a ketone group, an ester bond, or an amide bond.

The rings each may be an aromatic or non-aromatic hydrocarbon ring, an aromatic or non-aromatic heterocycle, or a polycyclic fused ring formed by a combination of two or more of these rings. The ring may be a three- to ten-membered ring, and is preferably a four- to eight-membered ring, more preferably a five- or six-membered ring.

Examples of the groups formed by bonding together any two or more of R_{1c} to R_{5c}, R_{6c} and R_{7c}, and Rₓ and R_{y} include alkylene groups such as a butylene group and a pentylene group. A methylene group in such an alkylene group may be substituted with a heteroatom such as an oxygen atom.

The groups formed by bonding together R_{5c} and R_{6c}, and R_{5c} and Rₓ are each preferably a single bond or an alkylene group. Examples of the alkylene group include a methylene group and an ethylene group.

R_{1c} to R_{5c}, R_{6c}, R_{7c}, Rₓ, R_{y}, and the rings formed by individually bonding together any two or more of R_{1c} to R_{5c}, R_{5c} and R_{6c}, R_{6c} and R_{7c}, R_{5c} and Rₓ, and Rₓ and R_{y} may have a substituent.

Next, the cation (ZaI-4b) will be described.

The cation (ZaI-4b) is a cation represented by a formula (ZaI-4b) below.

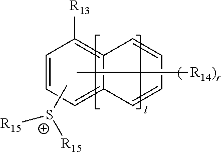

In the formula (ZaI-4b), 1 represents an integer of 0 to 2, and r represents an integer of 0 to 8.

R₁₃ represents a hydrogen atom, a halogen atom other than a fluorine atom (for example, a chlorine atom, a bromine atom, or an iodine atom), a hydroxy group, an alkyl group, an alkyl group substituted with a halogen atom other than a fluorine atom, an alkoxy group, a carboxyl group, an alkoxycarbonyl group, or a group including a cycloalkyl group (which may be a cycloalkyl group itself or a group including a cycloalkyl group as a part thereof). These groups may have a substituent.

R₁₄ represents a hydroxy group, a halogen atom other than a fluorine atom (for example, a chlorine atom, a bromine atom, or an iodine atom), an alkyl group, an alkyl group substituted with a halogen atom other than a fluorine atom, an alkoxy group, an alkoxycarbonyl group, an alkylcarbonyl group, an alkylsulfonyl group, a cycloalkylsulfonyl group, or a group including a cycloalkyl group (which may be a cycloalkyl group itself or a group including a cycloalkyl group as a part thereof). These groups may have a substituent. When a plurality of R₁₄'s are present, the plurality of R₁₄'s each independently represent the above-mentioned group, for example, a hydroxy group.

R₁₅'s each independently represent an alkyl group, a cycloalkyl group, or a naphthyl group. Two R₁₅'s may be bonded together to form a ring. When two R₁₅'s are bonded together to form a ring, the ring skeleton may include a heteroatom such as an oxygen atom or a nitrogen atom.

In one embodiment, two R₁₅'s are preferably alkylene groups and bonded together to form a ring structure. Note that the alkyl group, the cycloalkyl group, the naphthyl group, and the ring formed by bonding two R₁₅'s together may have a substituent.

In the formula (ZaI-4b), the alkyl groups in R₁₃, R₁₄, and R₁₅'s may be linear or branched. The number of carbon atoms of each of the alkyl groups is preferably 1 to 10. The alkyl group is preferably a methyl group, an ethyl group, a n-butyl group, a t-butyl group, or the like.

It is also preferable that substituents in R₁₃ to R₁₅ each independently form an acid-decomposable group by any combination of substituents.

Next, the formula (ZaII) will be described.

In the formula (ZaII), R²⁰⁴ and R²⁰⁵ each independently represent an aryl group, an alkyl group, or a cycloalkyl group.

The aryl group in R²⁰⁴ and R²⁰⁵ is preferably a phenyl group or a naphthyl group, more preferably a phenyl group. The aryl group in R²⁰⁴ and R²⁰⁵ may be an aryl group having a heterocycle having an oxygen atom, a nitrogen atom, a sulfur atom, or the like. Examples of the skeleton of the aryl group having a heterocycle include pyrrole, furan, thiophene, indole, benzofuran, and benzothiophene.

The alkyl group and the cycloalkyl group in R²⁰⁴ and R²⁰⁵ are preferably a linear alkyl group having 1 to 10 carbon atoms or a branched alkyl group having 3 to 10 carbon atoms (for example, a methyl group, an ethyl group, a propyl group, a butyl group, or a pentyl group), or a cycloalkyl group having 3 to 10 carbon atoms (for example, a cyclopentyl group, a cyclohexyl group, or a norbornyl group).

The aryl group, the alkyl group, and the cycloalkyl group in R²⁰⁴ and R²⁰⁵ may each independently have a substituent. Examples of the substituent that the aryl group, the alkyl group, and the cycloalkyl group in R²⁰⁴ and R²⁰⁵ may have include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms other than a fluorine atom, a hydroxy group, and a phenylthio group. It is also preferable that the substituents in R²⁰⁴ and R²⁰⁵ each independently form an acid-decomposable group by any combination of substituents.

Specific examples of the cation represented by M⁺ are shown below, but are not limited thereto.

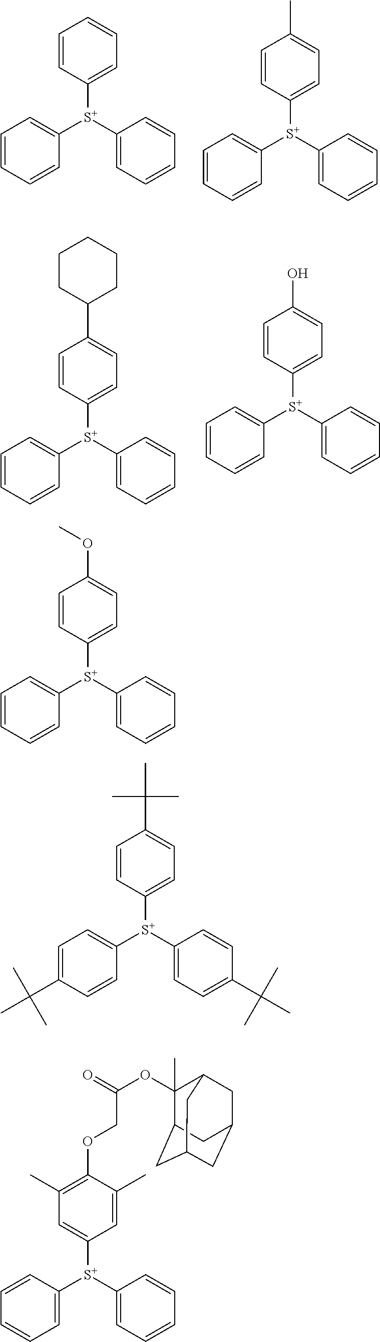

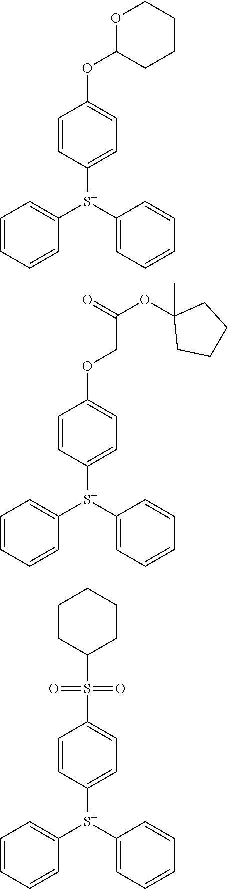 [SMILES: c1ccc([S+](c2ccccc2)c2ccc(OC3CCCCO3)cc2)cc1 ; CC1(OC(=O)COc2ccc([S+](c3ccccc3)c3ccccc3)cc2)CCCC1 ; O=S(=O)(c1ccc([S+](c2ccccc2)c2ccccc2)cc1)C1CCCCC1]

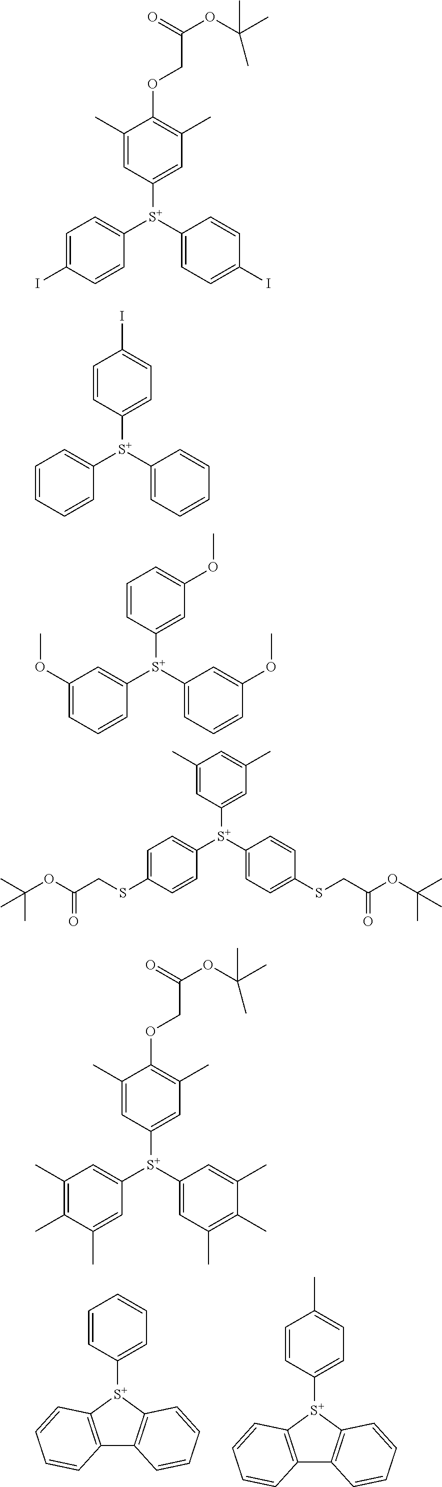

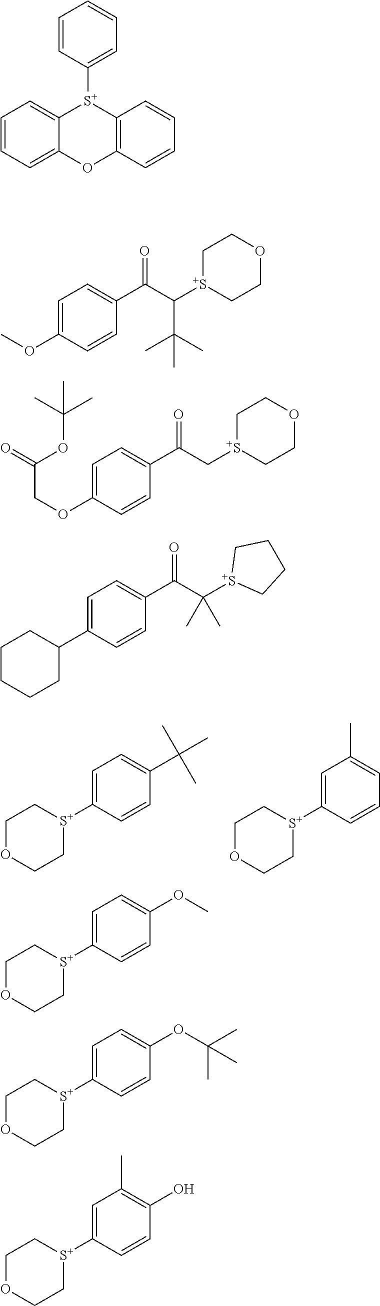

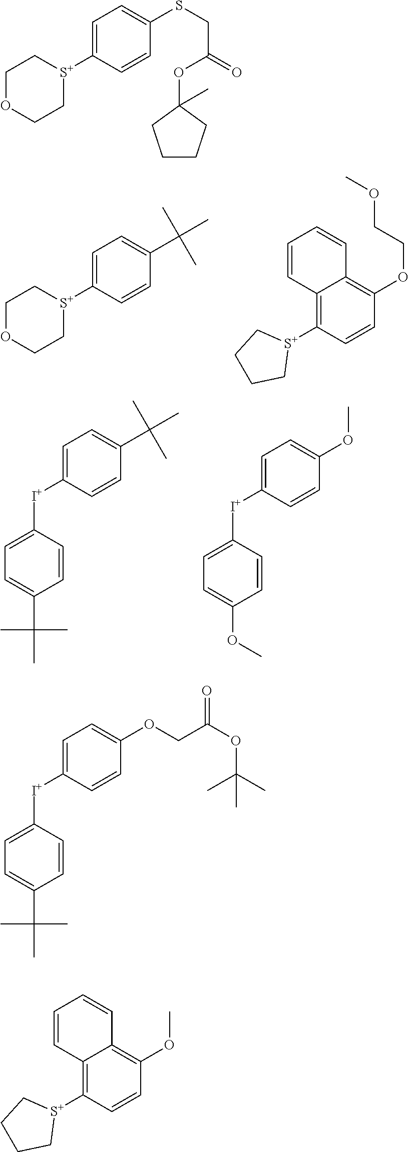

The compound (N) is preferably represented by a formula (a-2) below.

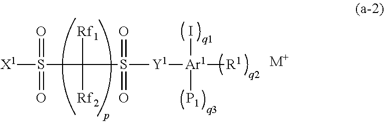

In the formula (a-2),
X¹ represents —O⁻ or —N⁻—SO₂—Rf₃.
Rf₁, Rf₂, and Rf₃ each independently represent a fluorine atom or an alkyl group having at least one fluorine atom. When a plurality of Rf₁'s and a plurality of Rf₂'s are present, the plurality of Rf₁'s may be the same or different and the plurality of Rf₂'s may be the same or different.
Y¹ represents a single bond, —O—, or —S—.
Ar¹ represents an aromatic group.
R¹ represents an organic group having an iodine atom.
p represents an integer of 1 or more.
q1 and q2 each independently represent an integer of 0 or more, provided that at least one of q1 or q2 represents an integer of 1 or more.
P¹ represents an organic group having no iodine atom.
q3 represents an integer of 0 or more.
M⁺ represents a cation.

The descriptions, specific examples, and preferred ranges of X¹, Rf₁, Rf₂, Rf₃, Y¹, p, and M⁺ in the formula (a-2) are the same as those of X¹, Rf₁, Rf₂, Rf₃, Y¹, p, and M⁺ in the above formula (a-1), respectively.

In the formula (a-2), Ar¹ represents an aromatic group.

The aromatic group represented by Ar¹ may be an aromatic hydrocarbon group (aryl group) or an aromatic heterocyclic group.

The aromatic group represented by Ar¹ may have a substituent other than an iodine atom and an organic group.

When Ar¹ represents an aryl group, the aryl group is preferably an aryl group having 6 to 30 carbon atoms, more preferably an aryl group having 6 to 20 carbon atoms, still more preferably a phenyl group, a naphthyl group, or an anthryl group, particularly preferably a phenyl group or a naphthyl group, most preferably a phenyl group.

When Ar¹ represents an aromatic heterocyclic group, the aromatic heterocyclic group preferably includes at least one heteroatom selected from the group consisting of a nitrogen atom, an oxygen atom, and a sulfur atom as a ring member. The number of ring member atoms of the aromatic heterocyclic group is not particularly limited, but is preferably 3 to 30, more preferably 4 to 20. The number of carbon atoms of the aromatic heterocyclic group is not particularly limited, but is preferably 2 to 20, more preferably 3 to 15.

Ar¹ preferably represents an aryl group.

In the formula (a-2), R¹ represents an organic group having an iodine atom.

The organic group represented by R¹ is not particularly limited except that it has an iodine atom (i.e., is substituted with an iodine atom). The number of carbon atoms of the organic group represented by R¹ is preferably 1 to 50, more preferably 1 to 40, still more preferably 1 to 30.

The organic group represented by R¹ may have a substituent other than an iodine atom.

The organic group represented by R¹ may or may not have an aromatic ring. The aromatic ring may be an aromatic hydrocarbon ring or an aromatic heterocycle, but is preferably an aromatic hydrocarbon ring. The number of carbon atoms of the aromatic hydrocarbon ring is preferably 6 to 20, more preferably 6 to 15, still more preferably 6 to 10. The aromatic heterocycle preferably includes at least one heteroatom selected from the group consisting of a nitrogen atom, an oxygen atom, and a sulfur atom as a ring member. The number of ring member atoms of the aromatic heterocycle is not particularly limited, but is preferably 3 to 30, more preferably 4 to 20. The number of carbon atoms of the aromatic heterocycle is not particularly limited, but is preferably 2 to 20, more preferably 3 to 15.

A preferred embodiment of the organic group represented by R¹ is one in which the organic group represented by R¹ has no aromatic ring.

The number of iodine atoms in the organic group represented by R¹ is 1 or more, preferably 1 to 8, more preferably 1 to 5, still more preferably 1 to 3.

When q2 in the formula (a-2) represents an integer of 1 or more, at least one R¹ preferably represents —O—R², —CO—O—R², —O—CO—R², —O—CO—O—R², —SO₂—R², or —SO₃—R², more preferably represents —CO—O—R², —O—CO—R², —O—CO—O—R², —SO₂—R², or —SO₃—R², still more preferably represents —CO—O—R², —O—CO—R², or —O—CO—O—R².

R² represents an organic group having an iodine atom.

The organic group represented by R² is not particularly limited except that it has an iodine atom (i.e., is substituted with an iodine atom). The number of carbon atoms of the organic group represented by R² is preferably 1 to 40, more preferably 1 to 30, still more preferably 1 to 20.

Examples of the organic group represented by R² include alkyl groups, alkenyl groups, alkynyl groups, cycloalkyl groups, aryl groups, and groups provided by combining two or more of these groups.

The organic group represented by R² is preferably an alkyl group, an alkenyl group, an alkynyl group, a cycloalkyl group, or a group provided by combining two or more of these groups, more preferably an alkyl group, a cycloalkyl group, or a group provided by combining two or more of these groups.

When the organic group represented by R² is an alkyl group and when the organic group represented by R² includes an alkyl group, the alkyl group may be linear or branched. The number of carbon atoms of the alkyl group is not particularly limited, and is, for example, preferably 1 to 20, more preferably 1 to 15, still more preferably 1 to 10. The alkyl group may have a substituent other than an iodine atom. The alkyl group may include an ether bond (—O—) in the chain. Examples of the alkyl group include a methyl group, an ethyl group, a propyl group, an isopropyl group, a n-butyl group, an isobutyl group, a sec-butyl group, a t-butyl group, a pentyl group, a neopentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group.

When the organic group represented by R² is an alkenyl group and when the organic group represented by R² includes an alkenyl group, the alkenyl group may be linear or branched. The number of carbon atoms of the alkenyl group is not particularly limited, and is, for example, preferably 2 to 20, more preferably 2 to 15, still more preferably 2 to 10. The alkenyl group may have a substituent other than an iodine atom. The alkenyl group may include an ether bond in the chain. Examples of the alkenyl group include a vinyl group and an allyl group.

When the organic group represented by R² is an alkynyl group and when the organic group represented by R² includes an alkynyl group, the alkynyl group may be linear or branched. The number of carbon atoms of the alkynyl group is not particularly limited, and is, for example, preferably 2 to 20, more preferably 2 to 15, still more preferably 2 to 10. The alkynyl group may have a substituent other than an iodine atom. The alkynyl group may include an ether bond in the chain. Examples of the alkynyl group include an ethynyl group.

When the organic group represented by R² is a cycloalkyl group and when the organic group represented by R² includes a cycloalkyl group, the cycloalkyl group may be monocyclic or polycyclic. The number of carbon atoms of the cycloalkyl group is not particularly limited, and is, for example, preferably 3 to 20, more preferably 4 to 15, still more preferably 5 to 10. The cycloalkyl group may have a substituent other than an iodine atom. In the cycloalkyl group, for example, one of methylene groups constituting the ring may be replaced by a heteroatom, such as an oxygen atom, a group having a heteroatom, such as a carbonyl group, or a vinylidene group. In the cycloalkyl group, one or more ethylene groups constituting the cycloalkane ring may each be replaced by a vinylene group. Examples of the cycloalkyl group include a cyclopentyl group, a cyclohexyl group, a norbornyl group, a tetracyclodecanyl group, a tetracyclododecanyl group, and an adamantyl group.

When the organic group represented by R² is an aryl group and when the organic group represented by R² includes an aryl group, the number of carbon atoms of the aryl group is not particularly limited, and is, for example, preferably 6 to 20, more preferably 6 to 15, still more preferably 6 to 10. The aryl group may have a substituent other than an iodine atom. Examples of the aryl group include a phenyl group, a naphthyl group, and an anthryl group.

The number of iodine atoms in the organic group represented by R² is 1 or more, preferably 1 to 8, more preferably 1 to 5, still more preferably 1 to 3.

In the formula (a-2), q1 and q2 each independently represent an integer of 0 or more, provided that at least one of q1 or q2 represents an integer of 1 or more.

q1 preferably represents an integer of 1 or more, more preferably represents an integer of 1 to 8, still more preferably represents an integer of 1 to 5, and particularly preferably represents an integer of 1 to 3.

q2 preferably represents an integer of 1 or more, more preferably represents an integer of 1 to 8, still more preferably represents an integer of 1 to 5, and particularly preferably represents an integer of 1 to 3.

In the formula (a-2), P¹ represents an organic group having no iodine atom.

The number of carbon atoms of the organic group represented by P¹ is preferably 1 to 50, more preferably 1 to 40, still more preferably 1 to 30.

The organic group represented by P¹ may have a substituent other than an iodine atom.

The organic group represented by P¹ may or may not have an aromatic ring. The aromatic ring may be an aromatic hydrocarbon ring or an aromatic heterocycle, but is preferably an aromatic hydrocarbon ring. The number of carbon atoms of the aromatic hydrocarbon ring is preferably 6 to 20, more preferably 6 to 15, still more preferably 6 to 10. The aromatic heterocycle preferably includes at least one heteroatom selected from the group consisting of a nitrogen atom, an oxygen atom, and a sulfur atom as a ring member. The number of ring member atoms of the aromatic heterocycle is not particularly limited, but is preferably 3 to 30, more preferably 4 to 20. The number of carbon atoms of the aromatic heterocycle is not particularly limited, but is preferably 2 to 20, more preferably 3 to 15.

The organic group represented by P¹ preferably does not have an aromatic ring.

When q3 in the formula (a-2) represents an integer of 1 or more, at least one P¹ preferably represents —O—P², —CO—O—P², —O—CO—P², —O—CO—O—P², —SO₂—P², or —SO₃—P², more preferably represents —CO—O—P², —O—CO—P², —O—CO—O—P², —SO₂—P², or —SO₃—P², and still more preferably represents —CO—O—P², —O—CO—P², or —O—CO—O—P².

P² represents an organic group having no iodine atom.

The number of carbon atoms of the organic group represented by P² is preferably 1 to 40, more preferably 1 to 30, still more preferably 1 to 20.

Examples of the organic group represented by P² include alkyl groups, alkenyl groups, alkynyl groups, cycloalkyl groups, aryl groups, and groups provided by combining two or more of these groups.

The organic group represented by P² is preferably an alkyl group, an alkenyl group, an alkynyl group, a cycloalkyl group, or a group provided by combining two or more of these groups, more preferably an alkyl group, a cycloalkyl group, or a group provided by combining two or more of these groups.

When the organic group represented by P² is an alkyl group and when the organic group represented by P² includes an alkyl group, the alkyl group may be linear or branched. The number of carbon atoms of the alkyl group is not particularly limited, and is, for example, preferably 1 to 20, more preferably 1 to 15, still more preferably 1 to 10. The alkyl group may have a substituent other than an iodine atom. The alkyl group may include an ether bond (—O—) in the chain. Examples of the alkyl group include a methyl group, an ethyl group, a propyl group, an isopropyl group, a n-butyl group, an isobutyl group, a sec-butyl group, a t-butyl group, a pentyl group, a neopentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group.

When the organic group represented by P² is an alkenyl group and when the organic group represented by P² includes an alkenyl group, the alkenyl group may be linear or branched. The number of carbon atoms of the alkenyl group is not particularly limited, and is, for example, preferably 2 to 20, more preferably 2 to 15, still more preferably 2 to 10. The alkenyl group may have a substituent other than an iodine atom. The alkenyl group may include an ether bond in the chain. Examples of the alkenyl group include a vinyl group and an allyl group.

When the organic group represented by P² is an alkynyl group and when the organic group represented by P² includes an alkynyl group, the alkynyl group may be linear or branched. The number of carbon atoms of the alkynyl group is not particularly limited, and is, for example, preferably 2 to 20, more preferably 2 to 15, still more preferably 2 to 10. The alkynyl group may have a substituent other than an iodine atom. The alkynyl group may include an ether bond in the chain. Examples of the alkynyl group include an ethynyl group.

When the organic group represented by P² is a cycloalkyl group and when the organic group represented by P² includes a cycloalkyl group, the cycloalkyl group may be monocyclic or polycyclic. The number of carbon atoms of the cycloalkyl group is not particularly limited, and is, for example, preferably 3 to 20, more preferably 4 to 15, still more preferably 5 to 10. The cycloalkyl group may have a substituent other than an iodine atom. In the cycloalkyl group, for example, one of methylene groups constituting the ring may be replaced by a heteroatom, such as an oxygen atom, a group having a heteroatom, such as a carbonyl group, or a vinylidene group. In the cycloalkyl group, one or more ethylene groups constituting the cycloalkane ring may each be replaced by a vinylene group. Examples of the cycloalkyl group include a cyclopentyl group, a cyclohexyl group, a norbornyl group, a tetracyclodecanyl group, a tetracyclododecanyl group, and an adamantyl group.

When the organic group represented by P² is an aryl group and when the organic group represented by P² includes an aryl group, the number of carbon atoms of the aryl group is not particularly limited, and is, for example, preferably 6 to 20, more preferably 6 to 15, still more preferably 6 to 10. The aryl group may have a substituent other than an iodine atom. Examples of the aryl group include a phenyl group, a naphthyl group, and an anthryl group.

In the formula (a-2), q3 represents an integer of 0 or more, more preferably represents an integer of 0 to 8, still more preferably represents an integer of 0 to 5, particularly preferably represents an integer of 0 to 3.

The molecular weight of the compound (N) is preferably 600 or more, more preferably 700 or more, still more preferably 800 or more. The molecular weight of the compound (N) is preferably 2,000 or less, more preferably 1,500 or less, still more preferably 1,300 or less.

The compound (N) can be synthesized on the basis of a publicly known method. A synthesis example of the compound (N) will be described in Examples described later.

Specific examples of the compound (N) include, but are not limited to, (I)-1 to (I)-22 used in Examples described later.

The content of the compound (N) in the composition of the present invention is not particularly limited, but is preferably 1 mass % or more, more preferably 5 mass % or more, still more preferably 10 mass % or more relative to the total solid contents of the composition of the present invention. The content of the compound (N) is preferably 50 mass % or less, more preferably 45 mass % or less, still more preferably 40 mass % or less relative to the total solid contents of the composition of the present invention.

Such compounds (N) may be used alone or in combination of two or more thereof. When two or more compounds (N) are used, the total content thereof is preferably within the above preferred content ranges.

### [Resin]

The composition of the present invention includes a resin.

The composition of the present invention preferably includes a resin whose polarity is increased by the action of an acid (also referred to as a “resin (A)”).

The resin (A) includes a group that is decomposed by the action of an acid to undergo an increase in polarity (also referred to as an “acid-decomposable group”), and preferably includes a repeating unit having an acid-decomposable group. In the case where the resin (A) has an acid-decomposable group, in the pattern forming method using the composition of the present invention, typically, when an alkali developer is used as the developer, a positive-type pattern is suitably formed, and when an organic-based developer is used as the developer, a negative-type pattern is suitably formed.

The repeating unit having an acid-decomposable group is preferably, in addition to the repeating unit having an acid-decomposable group, a repeating unit having an acid-decomposable group including an unsaturated bond.

### (Repeating Unit Having Acid-Decomposable Group)

The acid-decomposable group refers to a group that is decomposed by the action of an acid to generate a polar group. The acid-decomposable group preferably has a structure in which the polar group is protected with a group (leaving group) that leaves due to the action of an acid. That is, the resin (A) has a repeating unit having a group that is decomposed by the action of an acid to generate a polar group. In the resin having this repeating unit, the polarity is increased by the action of an acid, and thus the degree of solubility in an alkali developer increases, and the degree of solubility in an organic solvent decreases.

The polar group is preferably an alkali-soluble group, and examples thereof include acidic groups such as a carboxyl group, phenolic hydroxy groups, fluorinated alcohol groups, a sulfonic group, a phosphate group, a sulfonamide group, a sulfonylimide group, (alkylsulfonyl)(alkylcarbonyl)methylene groups, (alkylsulfonyl)(alkylcarbonyl)imide groups, bis(alkylcarbonyl)methylene groups, bis(alkylcarbonyl)imide groups, bis(alkylsulfonyl)methylene groups, bis(alkylsulfonyl)imide groups, tris(alkylcarbonyl)methylene groups, and tris(alkylsulfonyl)methylene groups, and alcoholic hydroxy groups.

Of these, the polar group is preferably a carboxyl group, a phenolic hydroxy group, a fluorinated alcohol group (preferably a hexafluoroisopropanol group), or a sulfonic group.

Examples of the group that leaves due to the action of an acid include groups represented by formulae (Y1) to (Y4).

—C(Rx₁)(Rx₂)(Rx₃) Formula (Y1):

—C(═O)OC(Rx₁)(Rx₂)(Rx₃) Formula (Y2):

—C(R₃₆)(R₃₇)(OR₃₈) Formula (Y3):

—C(Rn)(H)(Ar) Formula (Y4):

In the formula (Y1) and the formula (Y2), Rx₁ to Rx₃ each independently represent an alkyl group (linear or branched), a cycloalkyl group (monocyclic or polycyclic), an alkenyl group (linear or branched), or an aryl group (monocyclic or polycyclic). Note that, when Rx₁ to Rx₃ are all alkyl groups (linear or branched), at least two of Rx₁ to Rx₃ are preferably methyl groups.

In particular, it is preferable that Rx₁ to Rx₃ each independently represent a linear or branched alkyl group, and it is more preferable that Rx₁ to Rx₃ each independently represent a linear alkyl group.

Two of Rx₁ to Rx₃ may be bonded together to form a monocycle or a polycycle.

The alkyl group in Rx₁ to Rx₃ is preferably an alkyl group having 1 to 5 carbon atoms, such as a methyl group, an ethyl group, a n-propyl group, an isopropyl group, a n-butyl group, an isobutyl group, or a t-butyl group.

The cycloalkyl group in Rx₁ to Rx₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The aryl group in Rx₁ to Rx₃ is preferably an aryl group having 6 to 10 carbon atoms and may be, for example, a phenyl group, a naphthyl group, or an anthryl group.

The alkenyl group in Rx₁ to Rx₃ is preferably a vinyl group.

The ring formed by bonding two of Rx₁ to Rx₃ together is preferably a cycloalkyl group. The cycloalkyl group formed by bonding two of Rx₁ to Rx₃ together is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group, more preferably a monocyclic cycloalkyl group having 5 or 6 carbon atoms.

In the cycloalkyl group formed by bonding two of Rx₁ to Rx₃ together, one of methylene groups constituting the ring may be replaced by a heteroatom such as an oxygen atom, a group including a heteroatom, such as a carbonyl group, or a vinylidene group. In any of these cycloalkyl groups, one or more ethylene groups constituting the cycloalkane ring may each be replaced by a vinylene group.

In a preferred embodiment of the group represented by the formula (Y1) or the formula (Y²), for example, Rx₁ is a methyl group or an ethyl group, and Rx₂ and Rx₃ are bonded together to form the above-described cycloalkyl group.

When the actinic ray-sensitive or radiation-sensitive resin composition is, for example, a resist composition for EUV exposure, the alkyl group, the cycloalkyl group, the alkenyl group, and the aryl group represented by Rx₁ to Rx₃ and the ring formed by bonding two of Rx₁ to Rx₃ together also preferably further have, as a substituent, a fluorine atom or an iodine atom.

In the formula (Y3), R₃₆ to R₃₈ each independently represent a hydrogen atom or a monovalent organic group. R₃₇ and R₃₈ may be bonded together to form a ring. Examples of the monovalent organic group include alkyl groups, cycloalkyl groups, aryl groups, aralkyl groups, and alkenyl groups. It is also preferable that R₃₆ be a hydrogen atom.

The alkyl groups, cycloalkyl groups, aryl groups, and aralkyl groups each may include a heteroatom such as an oxygen atom and/or a group including a heteroatom, such as a carbonyl group. For example, in each of the above-mentioned alkyl groups, cycloalkyl groups, aryl groups, and aralkyl groups, one or more methylene groups may be substituted with a heteroatom such as an oxygen atom and/or a group including a heteroatom, such as a carbonyl group.

R₃₈ may be bonded to another substituent included in the main chain of the repeating unit to form a ring. The group formed by bonding R₃₈ and another substituent included in the main chain of the repeating unit is preferably an alkylene group such as a methylene group.

When the actinic ray-sensitive or radiation-sensitive resin composition is, for example, a resist composition for EUV exposure, the monovalent organic group represented by R₃₆ to R₃₈ and the ring formed by bonding R₃₇ and R₃₈ together also preferably further have, as a substituent, a fluorine atom or an iodine atom.

The formula (Y3) preferably represents a group represented by a formula (Y3-1) below.

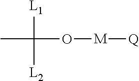

Here, L₁ and L₂ each independently represent a hydrogen atom, an alkyl group, a cycloalkyl group, an aryl group, or a combination thereof (for example, a combination of an alkyl group and an aryl group).

M represents a single bond or a divalent linking group.

Q represents an alkyl group that may include a heteroatom, a cycloalkyl group that may include a heteroatom, an aryl group that may include a heteroatom, an amino group, an ammonium group, a mercapto group, a cyano group, an aldehyde group, or a combination thereof (for example, a group provided by combining an alkyl group and a cycloalkyl group).

In the alkyl group and the cycloalkyl group, for example, one of methylene groups may be replaced by a heteroatom such as an oxygen atom or a group including a heteroatom, such as a carbonyl group.

Note that one of L₁ and L₂ is preferably a hydrogen atom, and the other is preferably an alkyl group, a cycloalkyl group, an aryl group, or a group that is a combination of an alkylene group and an aryl group.

At least two of Q, M, and L₁ may be bonded together to form a ring (preferably a five-membered or six-membered ring).

From the viewpoint of forming a finer pattern, L₂ is preferably a secondary or tertiary alkyl group, more preferably a tertiary alkyl group. Examples of the secondary alkyl group include an isopropyl group, a cyclohexyl group, and a norbornyl group, and examples of the tertiary alkyl group include a tert-butyl group and an adamantane group. In such embodiments, since Tg (glass transition temperature) and activation energy are high, the film hardness is ensured, and the occurrence of fogging can be suppressed.

When the actinic ray-sensitive or radiation-sensitive resin composition is, for example, a resist composition for EUV exposure, it is also preferable that the alkyl group, cycloalkyl group, aryl group, and combinations thereof, the groups being represented by L₁ and L₂, each further have a fluorine atom or an iodine atom as a substituent. It is also preferable that the alkyl group, the cycloalkyl group, the aryl group, and the aralkyl group include a heteroatom such as an oxygen atom in addition to a fluorine atom and an iodine atom. Specifically, in the alkyl group, the cycloalkyl group, the aryl group, and the aralkyl group, for example, one of methylene groups may be replaced by a heteroatom such as an oxygen atom or a group including a heteroatom, such as a carbonyl group.

When the actinic ray-sensitive or radiation-sensitive resin composition is, for example, a resist composition for EUV exposure, in the alkyl group that may include a heteroatom, the cycloalkyl group that may include a heteroatom, the aryl group that may include a heteroatom, the amino group, the ammonium group, the mercapto group, the cyano group, the aldehyde group, and a combination thereof, the groups being represented by Q, the heteroatom is also preferably a heteroatom selected from the group consisting of a fluorine atom, an iodine atom, and an oxygen atom.

In the formula (Y4), Ar represents an aromatic ring group. Rn represents an alkyl group, a cycloalkyl group, or an aryl group. Rn and Ar may be bonded together to form a non-aromatic ring. Ar is preferably an aryl group.

When the actinic ray-sensitive or radiation-sensitive resin composition is, for example, a resist composition for EUV exposure, it is also preferable that the aromatic ring group represented by Ar and the alkyl group, the cycloalkyl group, and the aryl group represented by Rn have a fluorine atom or an iodine atom as a substituent.

When, in the leaving group protecting the polar group, a non-aromatic ring is directly bonded to the polar group (or a residue thereof), it is also preferable that a ring member atom adjacent to the ring member atom directly bonded to the polar group (or a residue thereof) in the non-aromatic ring do not have a halogen atom such as a fluorine atom as a substituent, from the viewpoint that the repeating unit has excellent acid decomposability.

The group that leaves due to the action of an acid may be a 2-cyclopentenyl group having a substituent (e.g., an alkyl group), such as a 3-methyl-2-cyclopentenyl group; or a cyclohexyl group having a substituent (e.g., an alkyl group), such as a 1,1,4,4-tetramethylcyclohexyl group.

The repeating unit having an acid-decomposable group is also preferably a repeating unit represented by a formula (A).

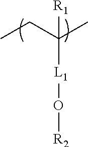

L₁ represents a divalent linking group that may have a fluorine atom or an iodine atom; R₁ represents a hydrogen atom, a fluorine atom, an iodine atom, an alkyl group that may have a fluorine atom or an iodine atom, or an aryl group that may have a fluorine atom or an iodine atom; and R₂ represents a leaving group that leaves due to the action of an acid and that may have a fluorine atom or an iodine atom. Note that at least one of L₁, R₁, or R₂ has a fluorine atom or an iodine atom.

Examples of the divalent linking group that may have a fluorine atom or an iodine atom and is represented by L₁ include —CO—, —O—, —S—, —SO—, —SO₂—, hydrocarbon groups that may have a fluorine atom or an iodine atom (for example, alkylene groups, cycloalkylene groups, alkenylene groups, and arylene groups), and linking groups provided by linking a plurality of these groups together. In particular, L₁ is preferably —CO—, an arylene group, or an -arylene group-fluorine or iodine atom-containing alkylene group-, more preferably —CO— or an -arylene group-fluorine or iodine atom-containing alkylene group-.

The arylene group is preferably a phenylene group.

The alkylene group may be linear or branched. The number of carbon atoms of the alkylene group is not particularly limited, but is preferably 1 to 10, more preferably 1 to 3.

The total number of fluorine atoms and iodine atoms included in the fluorine or iodine atom-containing alkylene group is not particularly limited, but is preferably 2 or more, more preferably 2 to 10, still more preferably 3 to 6.

The alkyl group represented by R₁ may be linear or branched. The number of carbon atoms of the alkyl group is not particularly limited, but is preferably 1 to 10, more preferably 1 to 3.

The total number of fluorine atoms and iodine atoms included in the fluorine or iodine atom-containing alkyl group represented by R₁ is not particularly limited, but is preferably 1 or more, more preferably 1 to 5, still more preferably 1 to 3.

The alkyl group represented by R₁ may include a heteroatom other than halogen atoms, such as an oxygen atom.

Examples of the leaving group that may have a fluorine atom or an iodine atom and is represented by R₂ include leaving groups that are represented by the formulae (Y1) to (Y4) and that have a fluorine atom or an iodine atom.

As the repeating unit having an acid-decomposable group, a repeating unit represented by a formula (AI) is also preferable.

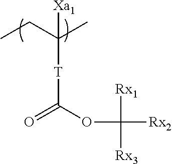

In the formula (AI), Xa₁ represents a hydrogen atom or an alkyl group that may have a substituent. T represents a single bond or a divalent linking group. Rx₁ to Rx₃ each independently represent an alkyl group (linear or branched), a cycloalkyl group (monocyclic or polycyclic), an alkenyl group (linear or branched), or an aryl group (monocyclic or polycyclic). Note that, when Rx₁ to Rx₃ are all alkyl groups (linear or branched), at least two of Rx₁ to Rx₃ are preferably methyl groups.

Two of Rx₁ to Rx₃ may be bonded together to form a monocyclic or polycyclic ring (such as a monocyclic or polycyclic cycloalkyl group).

The alkyl group that may have a substituent and is represented by Xa₁ may be, for example, a methyl group or a group represented by —CH₂—R₁₁. R₁₁ represents a halogen atom (such as a fluorine atom), a hydroxy group, or a monovalent organic group. Examples of the monovalent organic group represented by R₁₁ include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. The monovalent organic group represented by R₁₁ is preferably an alkyl group having 3 or less carbon atoms, more preferably a methyl group. Xa₁ is preferably a hydrogen atom, a methyl group, a trifluoromethyl group, or a hydroxymethyl group.

Examples of the divalent linking group in T include alkylene groups, aromatic ring groups, a —COO-Rt- group, and an —O-Rt- group. In the formulae, Rt represents an alkylene group or a cycloalkylene group.

T is preferably a single bond or a —COO-Rt- group. When T represents a —COO-Rt- group, Rt is preferably an alkylene group having 1 to 5 carbon atoms, more preferably a —CH₂— group, a —(CH₂)₂— group, or a —(CH₂)₃— group.

The alkyl group in Rx₁ to Rx₃ is preferably an alkyl group having 1 to 4 carbon atoms, such as a methyl group, an ethyl group, a n-propyl group, an isopropyl group, a n-butyl group, an isobutyl group, or a t-butyl group.

The cycloalkyl group in Rx₁ to Rx₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The aryl group in Rx₁ to Rx₃ is preferably an aryl group having 6 to 10 carbon atoms and may be, for example, a phenyl group, a naphthyl group, or an anthryl group.

The alkenyl group in Rx₁ to Rx₃ is preferably a vinyl group.

The cycloalkyl group formed by bonding two of Rx₁ to Rx₃ together is preferably a monocyclic cycloalkyl group such as a cyclopentyl group or a cyclohexyl group. A polycyclic cycloalkyl group such as a norbornyl group, a tetracyclodecanyl group, a tetracyclododecanyl group, or an adamantyl group is also preferred. Of these, a monocyclic cycloalkyl group having 5 to 6 carbon atoms is preferred.

In the cycloalkyl group formed by bonding two of Rx₁ to Rx₃ together, for example, one of methylene groups constituting the ring may be replaced by a heteroatom such as an oxygen atom, a group including a heteroatom, such as a carbonyl group, or a vinylidene group. In any of these cycloalkyl groups, one or more ethylene groups constituting the cycloalkane ring may each be replaced by a vinylene group.

In a preferred embodiment of the repeating unit represented by the formula (AI), for example, Rx₁ is a methyl group or an ethyl group, and Rx₂ and Rx₃ are bonded together to form the above-described cycloalkyl group.

When any of the above groups has a substituent, examples of the substituent include alkyl groups (having 1 to 4 carbon atoms), halogen atoms, a hydroxy group, alkoxy groups (having 1 to 4 carbon atoms), a carboxyl group, and alkoxycarbonyl groups (having 2 to 6 carbon atoms). The number of carbon atoms in the substituent is preferably 8 or less.

As the repeating unit represented by the formula (AI), an acid-decomposable tertiary alkyl (meth)acrylate-based repeating unit (a repeating unit in which Xa₁ represents a hydrogen atom or a methyl group, and T represents a single bond) is preferred.

The resin (A) may have a repeating unit having an acid-decomposable group including an unsaturated bond as the repeating unit having an acid-decomposable group.

The repeating unit having an acid-decomposable group including an unsaturated bond is preferably a repeating unit represented by a formula (B).

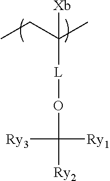

In the formula (B), Xb represents a hydrogen atom, a halogen atom, or an alkyl group that may have a substituent. L represents a single bond or a divalent linking group that may have a substituent. Ry₁ to Ry₃ each independently represent a linear or branched alkyl group, a monocyclic or polycyclic cycloalkyl group, an alkenyl group, an alkynyl group, or a monocyclic or polycyclic aryl group. Note that at least one of Ry₁ to Ry₃ represents an alkenyl group, an alkynyl group, a monocyclic or polycyclic cycloalkenyl group, or a monocyclic or polycyclic aryl group.

Two of Ry₁ to Ry₃ may be bonded together to form a monocyclic or polycyclic ring (such as a monocyclic or polycyclic cycloalkyl group or cycloalkenyl group).

The alkyl group that may have a substituent and is represented by Xb may be, for example, a methyl group or a group represented by —CH₂—R₁₁. R₁₁ represents a halogen atom (such as a fluorine atom), a hydroxy group, or a monovalent organic group, and examples thereof include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. R₁₁ is preferably an alkyl group having 3 or less carbon atoms, more preferably a methyl group. Xb is preferably a hydrogen atom, a fluorine atom, a methyl group, a trifluoromethyl group, or a hydroxymethyl group.

The divalent linking group in L may be an -Rt- group, a —CO— group, a —COO-Rt- group, a —COO-Rt-CO— group, an -Rt-CO— group, or an —O-Rt- group. In the formulae, Rt represents an alkylene group, a cycloalkylene group, or an aromatic ring group and is preferably an aromatic ring group.

L is preferably an -Rt- group, a —CO— group, a —COO-Rt-CO— group, or an -Rt-CO— group. Rt may have a substituent such as a halogen atom, a hydroxy group, or an alkoxy group.

The alkyl group in Ry₁ to Ry₃ is preferably an alkyl group having 1 to 4 carbon atoms such as a methyl group, an ethyl group, a n-propyl group, an isopropyl group, a n-butyl group, an isobutyl group, or a t-butyl group.

The cycloalkyl group in Ry₁ to Ry₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The aryl group in Ry₁ to Ry₃ is preferably an aryl group having 6 to 10 carbon atoms and may be, for example, a phenyl group, a naphthyl group, or an anthryl group.

The alkenyl group in Ry₁ to Ry₃ is preferably a vinyl group.

The alkynyl group in Ry₁ to Ry₃ is preferably an ethynyl group.

The cycloalkenyl group in Ry₁ to Ry₃ preferably has a structure in which a monocyclic cycloalkyl group, such as a cyclopentyl group or a cyclohexyl group, partially includes a double bond.

The cycloalkyl group formed by bonding two of Ry₁ to Ry₃ together is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group. Of these, a monocyclic cycloalkyl group having 5 to 6 carbon atoms is more preferred.

In the cycloalkyl group or the cycloalkenyl group formed by bonding two of Ry₁ to Ry₃ together, for example, one of methylene groups forming the ring may be replaced by a heteroatom, such as an oxygen atom, a group including a heteroatom, such as a carbonyl group, an —SO₂— group, or an —SO₃— group, a vinylidene group, or a combination thereof. In the cycloalkyl group or the cycloalkenyl group, one or more ethylene groups forming the cycloalkane ring or the cycloalkene ring may each be replaced by a vinylene group.

In a preferred embodiment of the repeating unit represented by the formula (B), for example, Ry₁ is a methyl group, an ethyl group, a vinyl group, an allyl group, or an aryl group, and Ry₂ and Ry₃ are bonded together to form the above-described cycloalkyl group or cycloalkenyl group.

When any of the above groups has a substituent, examples of the substituent include alkyl groups (having 1 to 4 carbon atoms), halogen atoms, a hydroxy group, alkoxy groups (having 1 to 4 carbon atoms), a carboxyl group, and alkoxycarbonyl groups (having 2 to 6 carbon atoms). The number of carbon atoms in the substituent is preferably 8 or less.

The repeating unit represented by the formula (B) is preferably an acid-decomposable (meth)acrylic acid tertiary ester-based repeating unit (a repeating unit in which Xb represents a hydrogen atom or a methyl group, and L represents a —CO— group), an acid-decomposable hydroxystyrene tertiary alkyl ether-based repeating unit (a repeating unit in which Xb represents a hydrogen atom or a methyl group, and L represents a phenylene group), or an acid-decomposable styrenecarboxylic acid tertiary ester-based repeating unit (a repeating unit in which Xb represents a hydrogen atom or a methyl group, and L represents an -Rt-CO— group (where Rt is an aromatic group)).

The content of the repeating unit having an acid-decomposable group including an unsaturated bond is preferably 15 mol % or more, more preferably 20 mol % or more, still more preferably 30 mol % or more relative to all the repeating units in the resin (A). The upper limit value of the content is preferably 80 mol % or less, more preferably 70 mol % or less, still more preferably 60 mol % or less relative to all the repeating units in the resin (A).

Specific examples of the repeating unit having an acid-decomposable group including an unsaturated bond include the repeating units described in [0067] to [0071] of WO2022/024928A. The above description is incorporated herein.

The following are specific examples of the repeating unit having an acid-decomposable group; however, the repeating unit having an acid-decomposable group is not limited thereto.

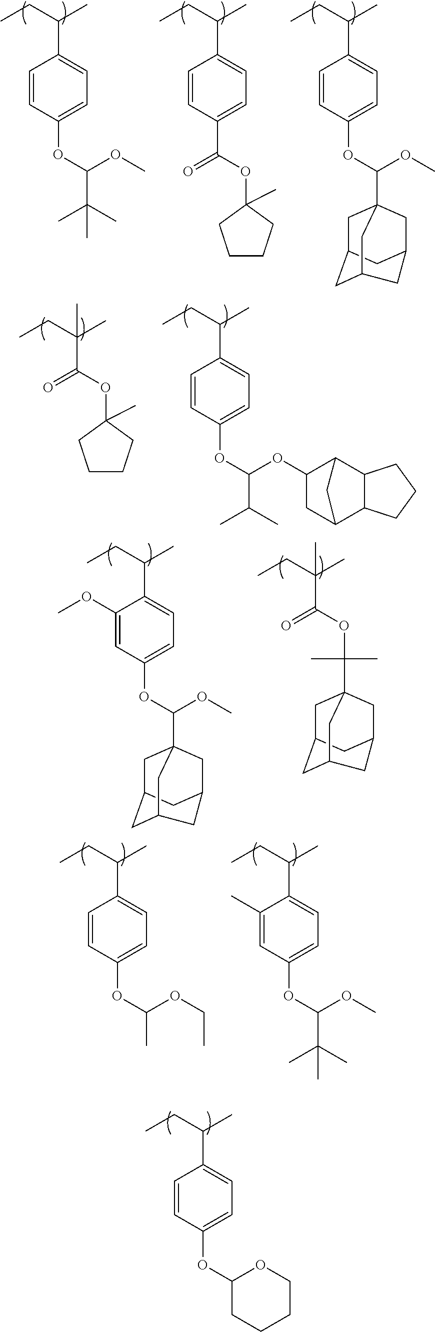

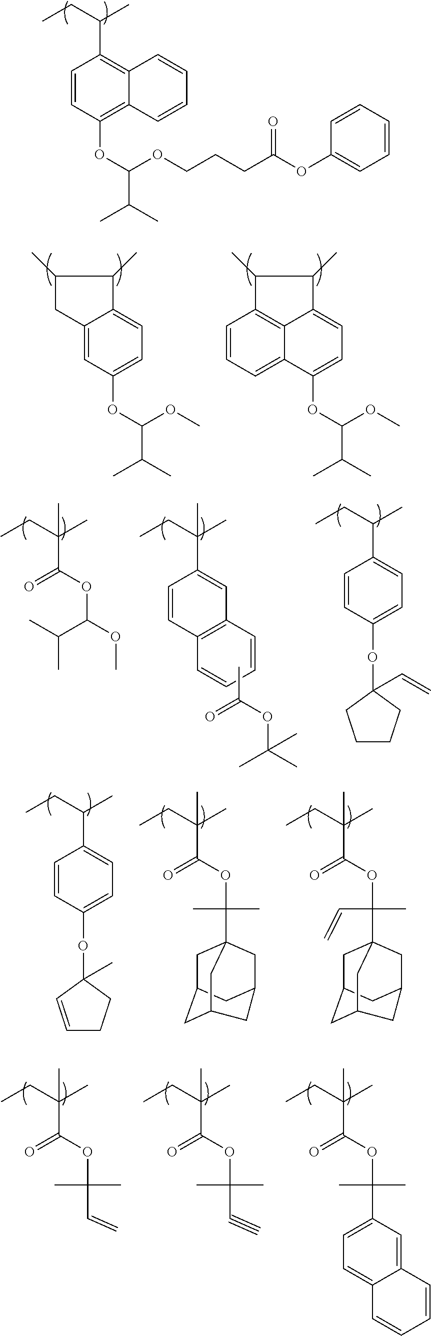

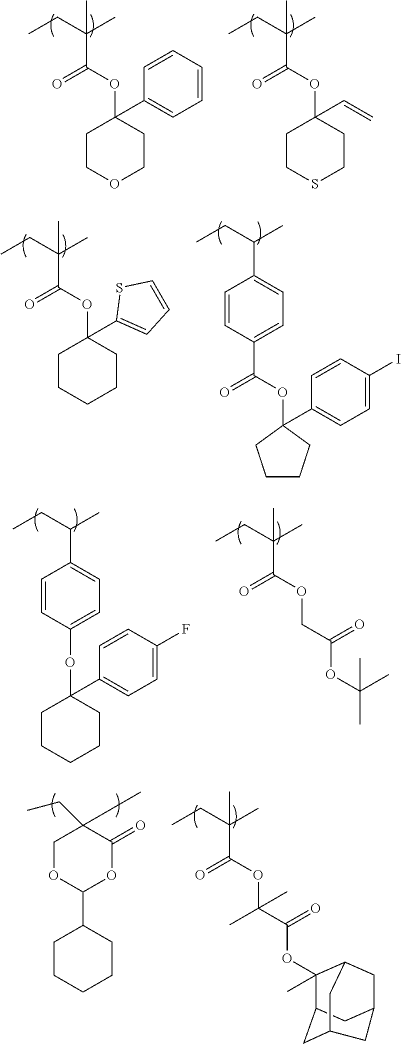

The content of the repeating unit having an acid-decomposable group is preferably 15 mol % or more, more preferably 20 mol % or more, still more preferably 30 mol % or more relative to all the repeating units in the resin (A). The upper limit value of the content is preferably 90 mol % or less, more preferably 80 mol % or less, still more preferably 70 mol % or less, particularly preferably 60 mol % or less relative to all the repeating units in the resin (A). The resin (A) may include at least one repeating unit selected from the group consisting of Group A below and/or at least one repeating unit selected from the group consisting of Group B below.

Group A: Group consisting of the following repeating units (20) to (25)
(20) A repeating unit having an acid group, which will be described later
(21) A repeating unit having neither an acid-decomposable group nor an acid group but having a fluorine atom, a bromine atom, or an iodine atom, which will be described later
(22) A repeating unit having a lactone group, a sultone group, or a carbonate group, which will be described later
(23) A repeating unit having a photoacid generating group, which will be described later
(24) A repeating unit represented by a formula (V-1) or a formula (V-2) below, which will be described later
(25) A repeating unit for reducing the mobility of the main chain

Note that repeating units represented by formulae (A) to (E), which will be described later, each correspond to the (25) repeating unit for reducing the mobility of the main chain.

Group B: Group consisting of the following repeating units (30) to (32)
(30) A repeating unit having at least one group selected from the group consisting of lactone groups, sultone groups, carbonate groups, a hydroxy group, a cyano group, and alkali-soluble groups, which will be described later
(31) A repeating unit that has an alicyclic hydrocarbon structure and that does not exhibit acid decomposability, which will be described later
(32) A repeating unit having neither a hydroxy group nor a cyano group and represented by a formula (III), which will be described later

The resin (A) preferably has an acid group and preferably includes a repeating unit having an acid group, as described later. The definition of the acid group will be described in a later part together with preferred embodiments of the repeating unit having an acid group. When the resin (A) has an acid group, a better interaction between the resin (A) and the acid generated from the photoacid generator is provided. As a result, the diffusion of the acid is further suppressed, and a pattern to be formed can have a more rectangular sectional shape.

The resin (A) may have at least one repeating unit selected from the group consisting of Group A described above. When the actinic ray-sensitive or radiation-sensitive resin composition is used as an actinic ray-sensitive or radiation-sensitive resin composition for EUV exposure, the resin (A) preferably has at least one repeating unit selected from the group consisting of Group A described above.

The resin (A) may include at least one of a fluorine atom or an iodine atom. When the actinic ray-sensitive or radiation-sensitive resin composition is used as an actinic ray-sensitive or radiation-sensitive resin composition for EUV exposure, the resin (A) preferably includes at least one of a fluorine atom or an iodine atom. When the resin (A) includes both a fluorine atom and an iodine atom, the resin (A) may have one type of repeating unit including both a fluorine atom and an iodine atom, or the resin (A) may include two types of repeating units including a repeating unit having a fluorine atom and a repeating unit including an iodine atom.

The resin (A) may have a repeating unit having an aromatic group. When the actinic ray-sensitive or radiation-sensitive resin composition is used as an actinic ray-sensitive or radiation-sensitive resin composition for EUV exposure, it is also preferable that the resin (A) have a repeating unit having an aromatic group.

The resin (A) may have at least one repeating unit selected from the group consisting of Group B described above. When the actinic ray-sensitive or radiation-sensitive resin composition is used as an actinic ray-sensitive or radiation-sensitive resin composition for ArF, the resin (A) preferably has at least one repeating unit selected from the group consisting of Group B described above.

When the actinic ray-sensitive or radiation-sensitive resin composition is used as an actinic ray-sensitive or radiation-sensitive resin composition for ArF, the resin (A) preferably includes neither a fluorine atom nor a silicon atom.

When the actinic ray-sensitive or radiation-sensitive resin composition is used as an actinic ray-sensitive or radiation-sensitive resin composition for ArF, the resin (A) preferably does not have an aromatic group.

### (Repeating Unit Having Acid Group)

The resin (A) may have a repeating unit having an acid group.

The acid group is preferably an acid group having a pKa of 13 or less. The acid dissociation constant of the acid group is preferably 13 or less, more preferably 3 to 13, still more preferably 5 to 10.

When the resin (A) has an acid group having a pKa of 13 or less, the content of the acid group in the resin (A) is not particularly limited but is often 0.2 to 6.0 mmol/g. In particular, the content of the acid group is preferably 0.8 to 6.0 mmol/g, more preferably 1.2 to 5.0 mmol/g, still more preferably 1.6 to 4.0 mmol/g. When the content of the acid group is within the above range, development proceeds satisfactorily, a pattern to be formed has a good shape, and thus high resolution is also achieved.

The acid group is preferably, for example, a carboxyl group, a phenolic hydroxy group, a fluorinated alcohol group (preferably a hexafluoroisopropanol group), a sulfonic group, a sulfonamide group, or an isopropanol group.

In the hexafluoroisopropanol group, one or more (preferably one or two) of the fluorine atoms may each be substituted with a group other than a fluorine atom (such as an alkoxycarbonyl group). The acid group is also preferably —C(CF₃)(OH)—CF₂— formed in this manner. Alternatively, one or more of the fluorine atoms may each be substituted with a group other than a fluorine atom to form a ring including —C(CF₃)(OH)—CF₂—.

The repeating unit having an acid group is preferably a repeating unit different from the above-described repeating unit having a structure in which a polar group is protected with a group that leaves due to the action of an acid and a repeating unit having a lactone group, a sultone group, or a carbonate group described later.

The repeating unit having an acid group may have a fluorine atom or an iodine atom.

Specific examples of the repeating unit having an acid group include the repeating units described in [0088] to [0089] and [0103] to [0110] of WO2022/024928A. The above description is incorporated herein.

The repeating unit having an acid group is preferably a repeating unit having a phenolic hydroxy group.

The repeating unit having a phenolic hydroxy group is preferably a repeating unit different from the above-described repeating unit having an acid-decomposable group.

The repeating unit having a phenolic hydroxy group is preferably a repeating unit represented by a formula (Pa2) below.

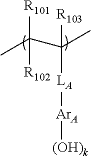

In the formula (Pa2), R₁₀₁, R₁₀₂, and R₁₀₃ each independently represent a hydrogen atom, an alkyl group, a cycloalkyl group, a halogen atom, a cyano group, or an alkoxycarbonyl group. R₁₀₂ may be bonded to Ar_{A} to form a ring, and R₁₀₂ in this case represents a single bond or an alkylene group.

L_{A} represents a single bond or a divalent linking group.

Ar_{A} represents an aromatic ring group.

k represents an integer of 1 to 5.

R₁₀₁, R₁₀₂, and R₁₀₃ in the formula (Pa2) each independently represent a hydrogen atom, an alkyl group, a cycloalkyl group, a halogen atom, a cyano group, or an alkoxycarbonyl group.

The alkyl group in R₁₀₁, R₁₀₂, and R₁₀₃ may be linear or branched. The number of carbon atoms of the alkyl group is not particularly limited, but is preferably 1 to 10, more preferably 1 to 5, particularly preferably 1 to 3. Examples of the alkyl group include a methyl group, an ethyl group, a n-propyl group, an isopropyl group, a n-butyl group, an isobutyl group, and a t-butyl group.

The number of carbon atoms of the cycloalkyl group in R₁₀₁, R₁₀₂, and R₁₀₃ is not particularly limited, but is preferably 3 to 20, more preferably 5 to 15. The cycloalkyl group in R₁₀₁, R₁₀₂, and R₁₀₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The halogen atom in R₁₀₁, R₁₀₂, and R₁₀₃ may be a fluorine atom, a chlorine atom, a bromine atom, or an iodine atom and is preferably a fluorine atom or an iodine atom.

The alkyl group included in the alkoxycarbonyl group in R₁₀₁, R₁₀₂, and R₁₀₃ may be linear or branched. The number of carbon atoms of the alkyl group included in the alkoxycarbonyl group is not particularly limited, but is preferably 1 to 5, more preferably 1 to 3.

Ar_{A} in the formula (Pa2) represents an aromatic ring group and more specifically represents a (k+1)-valent aromatic ring group. When k is 1, the divalent aromatic ring group is preferably, for example, an arylene group having 6 to 18 carbon atoms, such as a phenylene group, a tolylene group, a naphthylene group, or an anthracenylene group, or a divalent aromatic ring group including a heterocycle such as a thiophene ring, a furan ring, a pyrrole ring, a benzothiophene ring, a benzofuran ring, a benzopyrrole ring, a triazine ring, an imidazole ring, a benzimidazole ring, a triazole ring, a thiadiazole ring, or a thiazole ring. The aromatic ring group may have a substituent.

When k is an integer of 2 or more, specific examples of the (k+1)-valent aromatic ring group include groups provided by removing any (k−1) hydrogen atoms from the foregoing specific examples of the divalent aromatic ring group.

The (k+1)-valent aromatic ring group may further have a substituent.

The substituent that the (k+1)-valent aromatic ring group may have is not particularly limited, and examples thereof include alkyl groups such as a methyl group, an ethyl group, a propyl group, an isopropyl group, a n-butyl group, a sec-butyl group, a hexyl group, a 2-ethylhexyl group, an octyl group, and a dodecyl group; alkoxy groups such as a methoxy group, an ethoxy group, a hydroxyethoxy group, a propoxy group, a hydroxypropoxy group, and a butoxy group; and aryl groups such as a phenyl group.

Ar_{A} preferably represents an aromatic ring group having 6 to 18 carbon atoms, and more preferably represents a benzene ring group, a naphthalene ring group, or a biphenylene ring group.

L_{A} in the formula (Pa2) represents a single bond or a divalent linking group.

Examples of the divalent linking group represented by L_{A} include, but are not particularly limited to, —COO—, —CONR₁₀₄—, alkylene groups, and groups provided by combining two or more of these groups. R₁₀₄ above represents a hydrogen atom or an alkyl group.

The alkylene group is not particularly limited, but is preferably an alkylene group having 1 to 8 carbon atoms, such as a methylene group, an ethylene group, a propylene group, a butylene group, a hexylene group, or an octylene group.

When R₁₀₄ represents an alkyl group, the alkyl group may be an alkyl group having 20 or less carbon atoms, such as a methyl group, an ethyl group, a propyl group, an isopropyl group, a n-butyl group, a sec-butyl group, a hexyl group, a 2-ethylhexyl group, an octyl group, or a dodecyl group and is preferably an alkyl group having 8 or less carbon atoms.

The repeating unit represented by the formula (Pa2) preferably includes a hydroxystyrene structure. That is, Ar_{A} preferably represents a benzene ring group.

k preferably represents an integer of 1 to 3 and more preferably represents 1 or 2.

The following are specific examples of the repeating unit having an acid group; however, the repeating unit having an acid group is not limited thereto. In the following structural formulae, G¹ and G² each independently represent a hydrogen atom, a methyl group, a fluorine atom, a chlorine atom, a trifluoromethyl group, a cyano group, a hydroxy group, or a hydroxymethyl group. f1 represents an integer of 1 to 3.

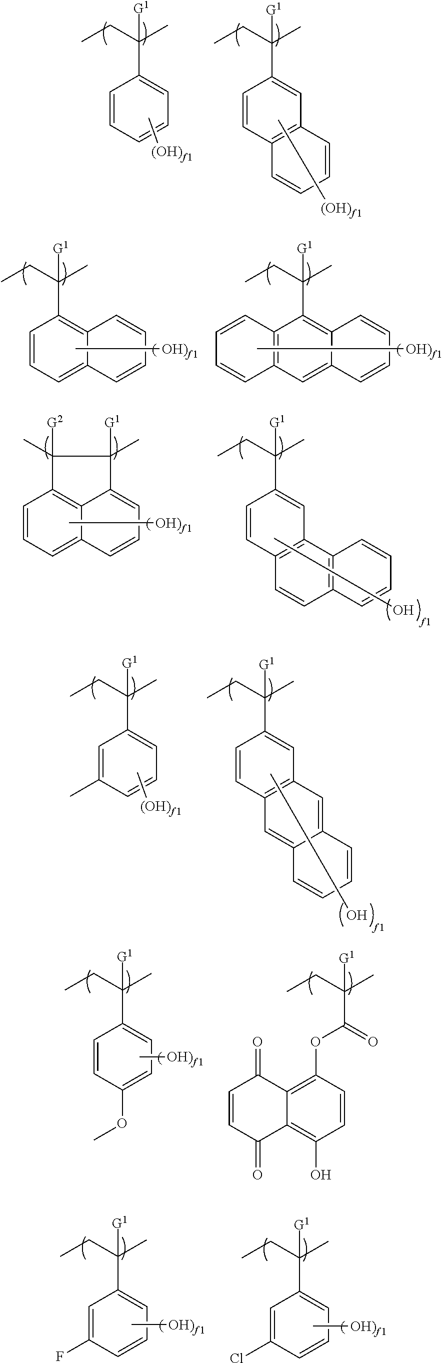

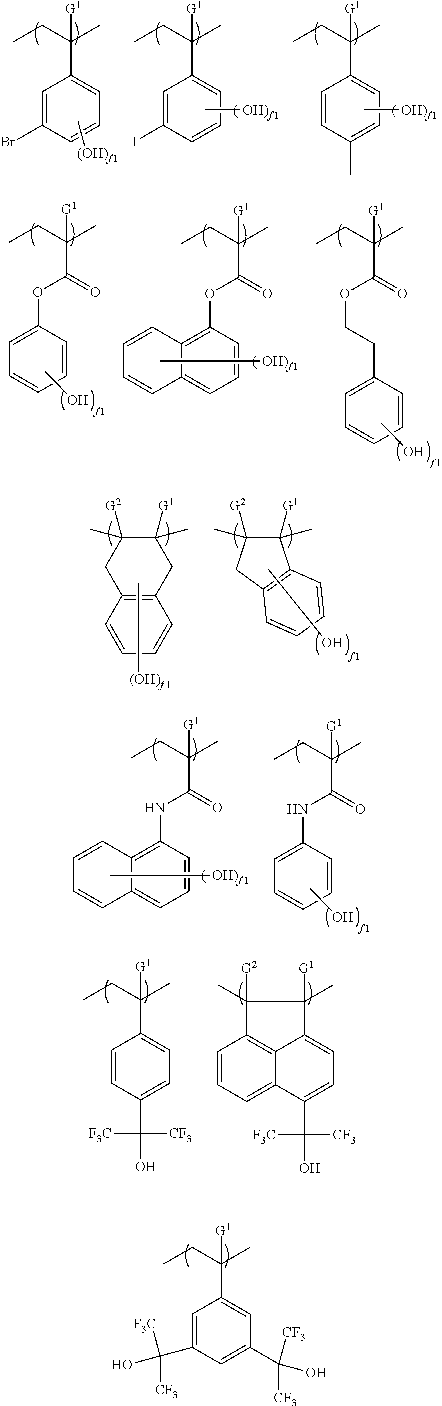

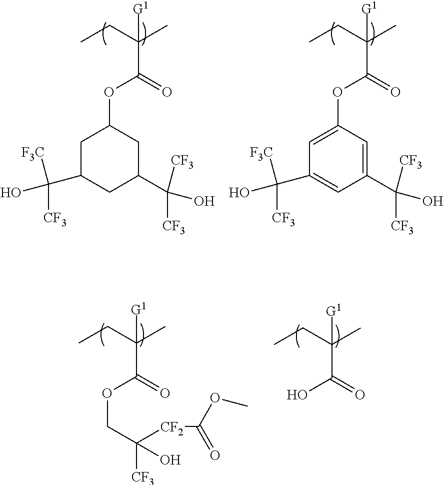

When the resin (A) includes a repeating unit having an acid group, the content of the repeating unit having an acid group is preferably 10 mol % or more, more preferably 15 mol % or more relative to all the repeating units in the resin (A). The upper limit value of the content is preferably 70 mol % or less, more preferably 65 mol % or less, still more preferably 60 mol % or less relative to all the repeating units in the resin (A).

### (Repeating Unit Having Neither Acid-Decomposable Group Nor Acid Group but Having Fluorine Atom, Bromine Atom, or Iodine Atom)

The resin (A) may have a repeating unit having neither an acid-decomposable group nor an acid group but having a fluorine atom, a bromine atom, or an iodine atom (hereinafter, also referred to as a unit X), in addition to the <Repeating unit having acid-decomposable group> and <Repeating unit having acid group> described above. The <Repeating unit having neither acid-decomposable group nor acid group but having fluorine atom, bromine atom, or iodine atom> referred to here is preferably different from other types of repeating units belonging to Group A, such as the <Repeating unit having lactone group, sultone group, or carbonate group> and the <Repeating unit having photoacid generating group>, which will be described later.

The unit X is preferably a repeating unit represented by a formula (C).

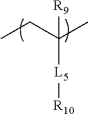

L₅ represents a single bond or an ester group. R₉ represents a hydrogen atom or an alkyl group that may have a fluorine atom or an iodine atom. R₁₀ represents a hydrogen atom, an alkyl group that may have a fluorine atom or an iodine atom, a cycloalkyl group that may have a fluorine atom or an iodine atom, an aryl group that may have a fluorine atom or an iodine atom, or a group provided by combining any of these groups.

Specific examples of repeating units having a fluorine atom or an iodine atom include the repeating units described in paragraphs [0116] to [0117] of WO2022/024928A. The above description is incorporated herein.

The content of the unit X is preferably 0 mol % or more, more preferably 5 mol % or more, still more preferably 10 mol % or more relative to all the repeating units in the resin (A). The upper limit value of the content is preferably 50 mol % or less, more preferably 45 mol % or less, still more preferably 40 mol % or less relative to all the repeating units in the resin (A).

Among the repeating units of the resin (A), the total content of repeating units including at least one of a fluorine atom, a bromine atom, or an iodine atom is preferably 10 mol % or more, more preferably 20 mol % or more, still more preferably 30 mol % or more, particularly preferably 40 mol % or more relative to all the repeating units in the resin (A). The upper limit value is not particularly limited, but is, for example, 100 mol % or less relative to all the repeating units in the resin (A).

Examples of the repeating units including at least one of a fluorine atom, a bromine atom, or an iodine atom include a repeating unit having a fluorine atom, a bromine atom, or an iodine atom and having an acid-decomposable group, a repeating unit having a fluorine atom, a bromine atom, or an iodine atom and having an acid group, and a repeating unit having a fluorine atom, a bromine atom, or an iodine atom.

### (Repeating Unit Having Lactone Group, Sultone Group, or Carbonate Group)

The resin (A) may have a repeating unit having at least one selected from the group consisting of a lactone group, a sultone group, and a carbonate group (hereinafter, also referred to as a “unit Y”).

It is also preferable that the unit Y do not have an acid group such as a hydroxy group or a hexafluoropropanol group.

The lactone group or the sultone group at least has a lactone structure or a sultone structure. The lactone structure or the sultone structure is preferably a five- to seven-membered lactone structure or a five- to seven-membered sultone structure. In particular, a five- to seven-membered lactone structure to which another ring structure is fused so as to form a bicyclo structure or a spiro structure, or a five- to seven-membered sultone structure to which another ring structure is fused so as to form a bicyclo structure or a spiro structure is more preferred.

The resin (A) preferably has a repeating unit having a lactone group, a sultone group, or a carbonate group provided by abstracting one or more hydrogen atoms from ring member atoms of a lactone structure represented by any one of formulae (LC1-1) to (LC1-22) below, a sultone structure represented by any one of formulae (SL1-1) to (SL1-3) below, or a cyclic carbonate ester structure represented by any one of formulae (CC1-1) and (CC1-2) below, and the lactone group, the sultone group, or the carbonate group may be directly bonded to the main chain. For example, a ring member atom of the lactone group, the sultone group, or the carbonate group may constitute the main chain of the resin (A). The lactone group, the sultone group, and the carbonate group may have substituents.

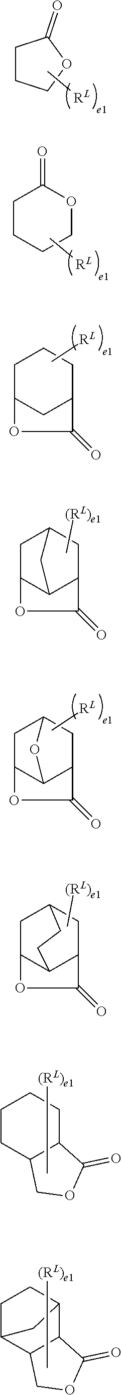

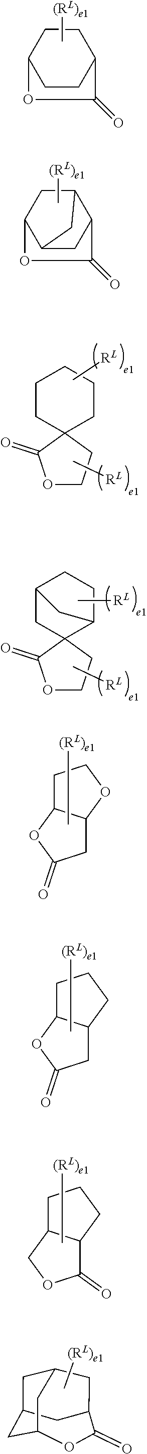

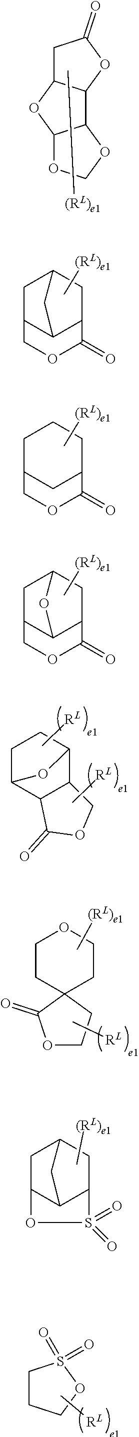

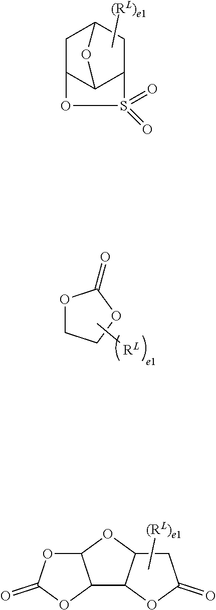

R^{L} in the above structural formulae represents a substituent. When a plurality of R^{L}'s are present, the plurality of R^{L}'s may be the same or different. Preferred examples of R^{L} include alkyl groups having 1 to 8 carbon atoms, cycloalkyl groups having 4 to 7 carbon atoms, alkoxy groups having 1 to 8 carbon atoms, alkoxycarbonyl groups having 1 to 8 carbon atoms, a carboxyl group, halogen atoms, a cyano group, and acid-decomposable groups. e1 represents an integer of 0 to 4. When e1 is 2 or more, a plurality of R^{L}'s present may be different from each other, and the plurality of R^{L}'s present may be bonded together to form a ring.

The repeating unit having a group including the lactone structure represented by any one of the formulae (LC1-1) to (LC1-22), the sultone structure represented by any one of the formulae (SL1-1) to (SL1-3), or the cyclic carbonate ester structure represented by any one of the formulae (CC1-1) and (CC1-2) may be, for example, a repeating unit represented by a formula (AI-2) below.

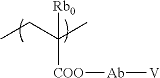

In the formula (AI-2), Rb₀ represents a hydrogen atom, a halogen atom, or an alkyl group having 1 to 4 carbon atoms. A preferred substituent that the alkyl group in Rb₀ may have may be a hydroxy group or a halogen atom.

Examples of the halogen atom in Rb₀ include a fluorine atom, a chlorine atom, a bromine atom, and an iodine atom. Rb₀ is preferably a hydrogen atom or a methyl group.

Ab represents a single bond, an alkylene group, a divalent linking group having a monocyclic or polycyclic alicyclic hydrocarbon structure, an ether group, an ester group, a carbonyl group, a carboxyl group, or a divalent linking group provided by combining any of these groups. Among these, Ab is preferably a single bond or a linking group represented by -Ab₁-CO₂—. Ab₁ is a linear or branched alkylene group or a monocyclic or polycyclic cycloalkylene group and is preferably a methylene group, an ethylene group, a cyclohexylene group, an adamantylene group, or a norbornylene group.

V represents a group provided by abstracting one hydrogen atom from a ring member atom of the lactone structure represented by any one of the formulae (LC1-1) to (LC1-22), a group provided by abstracting one hydrogen atom from a ring member atom of the sultone structure represented by any one of the formulae (SL1-1) to (SL1-3), or a group provided by abstracting one hydrogen atom from a ring member atom of the cyclic carbonate ester structure represented by any one of the formulae (CC1-1) and (CC1-2).

When the repeating unit having a lactone group or a sultone group has an optical isomer, any optical isomer may be used. A single optical isomer may be used alone, or a mixture of a plurality of optical isomers may be used. When a single optical isomer is mainly used, its optical purity (ee) is preferably 90 or more, more preferably 95 or more.

The carbonate group is preferably a cyclic carbonate ester group.

As the repeating unit having a cyclic carbonate ester group, for example, the description in [0127] to [0133] of WO2022/024928A can be referred to. The above description is incorporated herein.

When the resin (A) includes the unit Y, the content of the unit Y is preferably 1 mol % or more, more preferably 10 mol % or more relative to all the repeating units in the resin (A). The upper limit value of the content is preferably 85 mol % or less, more preferably 80 mol % or less, still more preferably 70 mol % or less, particularly preferably 60 mol % or less relative to all the repeating units in the resin (A).

### (Repeating Unit Having Photoacid Generating Group)

The resin (A) may have a repeating unit having a group that generates an acid upon irradiation with actinic rays or radiation (also referred to as a “photoacid generating group”) as a repeating unit other than the above repeating units.

The repeating unit having a photoacid generating group may be a repeating unit represented by a formula (4).

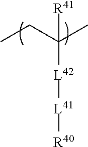

R⁴¹ represents a hydrogen atom or a methyl group. L⁴¹ represents a single bond or a divalent linking group. L⁴² represents a divalent linking group. R⁴⁰ represents a structural moiety that is decomposed by irradiation with actinic rays or radiation to generate an acid in the side chain.

Specific examples of the repeating unit having a photoacid generating group include the repeating units described in [0094] to [0105] of JP2014-041327A, the repeating units described in [0094] of WO2018/193954A, and the repeating units described in [0138] of WO2022/024928A. The above descriptions are incorporated herein.

Examples of the repeating unit represented by the formula (4) include the repeating units described in paragraphs [0094] to [0105] of JP2014-041327A and the repeating units described in paragraph [0094] of WO2018/193954A.

The content of the repeating unit having a photoacid generating group is preferably 1 mol % or more, more preferably 5 mol % or more relative to all the repeating units in the resin (A). The upper limit value of the content is preferably 40 mol % or less, more preferably 35 mol % or less, still more preferably 30 mol % or less relative to all the repeating units in the resin (A).

### (Repeating Unit Represented by Formula (V-1) or Formula (V-2) Below)

The resin (A) may have a repeating unit represented by a formula (V-1) below or a formula (V-2) below.

The repeating units represented by the formula (V-1) below and the formula (V-2) below are preferably repeating units different from the repeating units described above.

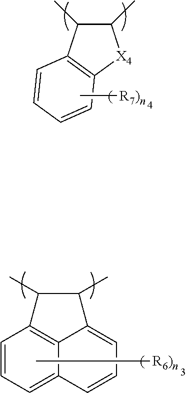

In the formulae,

R₆ and R₇ each independently represent a hydrogen atom, a hydroxy group, an alkyl group, an alkoxy group, an acyloxy group, a cyano group, a nitro group, an amino group, a halogen atom, an ester group (—OCOR or —COOR: R represents an alkyl group or fluorinated alkyl group having 1 to 6 carbon atoms), or a carboxyl group. The alkyl group is preferably a linear, branched, or cyclic alkyl group having 1 to 10 carbon atoms.
n₃ represents an integer of 0 to 6.
n₄ represents an integer of 0 to 4.
X₄ is a methylene group, an oxygen atom, or a sulfur atom.

Examples of the repeating unit represented by the formula (V-1) or (V-2) are as follows.

Examples of the repeating unit represented by the formula (V-1) or (V-2) include the repeating units described in paragraph [0100] of WO2018/193954A.

### (Repeating Unit for Reducing Mobility of Main Chain)

The resin (A) preferably has a high glass transition temperature (Tg) from the viewpoint that excessive diffusion of the generated acid or pattern collapse during development can be suppressed. Tg is preferably higher than 90 °C, more preferably higher than 100 °C, still more preferably higher than 110 °C, particularly preferably higher than 125 °C. Tg is preferably 400 °C or lower, more preferably 350 °C or lower from the viewpoint of a high dissolution rate in a developer.

In the present specification, the glass transition temperature (Tg) of a polymer such as the resin (A) (hereinafter, “Tg of repeating units”) is calculated by the following method. First, Tg's of homopolymers composed only of the repeating units included in the polymer are calculated by the Bicerano method. Next, the mass ratios (%) of the repeating units relative to all the repeating units in the polymer are calculated. Next, Tg's at the mass ratios are calculated using the Fox equation (described in, for example, Materials Letters 62 (2008) 3152), and the sum total of the Tg's is defined as the Tg (°C) of the polymer.

The Bicerano method is described in Prediction of polymer properties, Marcel Dekker Inc., New York (1993). The calculation of Tg by the Bicerano method can be performed using software for estimating physical properties of polymers, MDL Polymer (MDL Information Systems, Inc.).

In order to increase Tg of the resin (A) (preferably, to make Tg higher than 90 °C), the mobility of the main chain of the resin (A) is preferably reduced. Examples of the method for reducing the mobility of the main chain of the resin (A) include the following methods (a) to (e):
(a) Introduction of a bulky substituent into the main chain
(b) Introduction of a plurality of substituents into the main chain
(c) Introduction of a substituent that induces an interaction between molecules of the resin (A) into the vicinity of the main chain
(d) Formation of the main chain with a ring structure
(e) Linking of a ring structure to the main chain

The resin (A) preferably has a repeating unit whose homopolymer has a Tg of 130 °C or higher.

The type of the repeating unit whose homopolymer has a Tg of 130 °C or higher is not particularly limited as long as it is a repeating unit whose homopolymer has a Tg of 130 °C or higher as calculated by the Bicerano method. Note that repeating units represented by a formula (A) to a formula (E) described below may each correspond to the repeating unit whose homopolymer has a Tg of 130 °C or higher, although it depends on the types of functional groups in the repeating units.

An example of specific means for achieving (a) above is a method of introducing a repeating unit represented by a formula (A) into the resin (A).

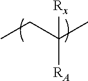

In the formula (A), RA represents a group including a polycyclic structure. Rₓ represents a hydrogen atom, a methyl group, or an ethyl group. The group including a polycyclic structure is a group including a plurality of ring structures, and the plurality of ring structures may be fused together or may not be fused together.

Specific examples of the repeating unit represented by the formula (A) include those described in paragraphs [0107] to [0119] of WO2018/193954A.

An example of specific means for achieving (b) above is a method of introducing a repeating unit represented by a formula (B) into the resin (A).

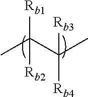

In the formula (B), R_{b1} to R_{b4} each independently represent a hydrogen atom or an organic group, and at least two or more of R_{b1} to R_{b4} represent organic groups.

When at least one of the organic groups is a group whose ring structure is directly linked to the main chain in the repeating unit, the types of other organic groups are not particularly limited.

When none of the organic groups is a group whose ring structure is directly linked to the main chain in the repeating unit, at least two or more of the organic groups are substituents in which the number of constituent atoms excluding hydrogen atoms is 3 or more.

Specific examples of the repeating unit represented by the formula (B) include those described in paragraphs [0113] to [0115] of WO2018/193954A.

An example of specific means for achieving (c) above is a method of introducing a repeating unit represented by a formula (C) into the resin (A).

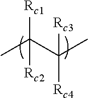

In the formula (C), R_{c1} to R_{c4} each independently represent a hydrogen atom or an organic group, and at least one of R_{c1} to R_{c4} is a group including a hydrogen-bond-forming hydrogen atom positioned within three atoms from a carbon atom in the main chain. In particular, from the viewpoint of inducing the interaction between the main chains of molecules of the resin (A), it is preferable to have a hydrogen-bond-forming hydrogen atom positioned within two atoms (closer to the main chain).

Specific examples of the repeating unit represented by the formula (C) include those described in paragraphs [0119] to [0121] of WO2018/193954A.

An example of specific means for achieving (d) above is a method of introducing a repeating unit represented by a formula (D) into the resin (A).

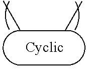

In the formula (D), “Cyclic” represents a group that forms the main chain with a ring structure. The number of atoms constituting the ring is not particularly limited.

Specific examples of the repeating unit represented by the formula (D) include those described in paragraphs [0126] to [0127] of WO2018/193954A.

An example of specific means for achieving (e) above is a method of introducing a repeating unit represented by a formula (E) into the resin (A).

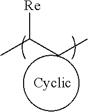

In the formula (E), each Re independently represents a hydrogen atom or an organic group. The organic group may be, for example, an alkyl group, cycloalkyl group, aryl group, aralkyl group, or alkenyl group that may have a substituent.

“Cyclic” is a cyclic group including a carbon atom of the main chain. The number of atoms included in the cyclic group is not particularly limited.

Specific examples of the repeating unit represented by the formula (E) include those described in paragraphs [0131] to [0133] of WO2018/193954A.

(Repeating Unit Having at Least One Group Selected from the Group Consisting of Lactone Groups, Sultone Groups, Carbonate Groups, Hydroxy Group, Cyano Group, and Alkali-Soluble Groups)

The resin (A) may have a repeating unit having at least one group selected from the group consisting of lactone groups, sultone groups, carbonate groups, a hydroxy group, a cyano group, and alkali-soluble groups.

Examples of the repeating unit that has a lactone group, a sultone group, or a carbonate group and that the resin (A) has include the repeating units described in the <Repeating unit having lactone group, sultone group, or carbonate group> above. The preferred content of the repeating unit is also as described in the <Repeating unit having lactone group, sultone group, or carbonate group> above.

The resin (A) may have a repeating unit having a hydroxy group or a cyano group. This results in improvement in adhesiveness to a substrate and affinity for a developer.

The repeating unit having a hydroxy group or a cyano group is preferably a repeating unit having an alicyclic hydrocarbon structure substituted with a hydroxy group or a cyano group.

Preferably, the repeating unit having a hydroxy group or a cyano group has no acid-decomposable group. Examples of the repeating unit having a hydroxy group or a cyano group include those described in paragraphs [0081] to [0084] of JP2014-098921A.

The resin (A) may have a repeating unit having an alkali-soluble group.

Examples of the alkali-soluble group include a carboxyl group, a sulfonamide group, a sulfonylimide group, a bissulfonylimide group, and aliphatic alcohol groups substituted with an electron-withdrawing group at the α-position (for example, a hexafluoroisopropanol group), and the alkali-soluble group is preferably a carboxyl group. When the resin (A) includes a repeating unit having an alkali-soluble group, the resolution is increased in contact hole applications. Examples of the repeating unit having an alkali-soluble group include those described in paragraphs [0085] and [0086] of JP2014-098921A.

(Repeating Unit that has Alicyclic Hydrocarbon Structure and that does not Exhibit Acid Decomposability)

The resin (A) may have a repeating unit that has an alicyclic hydrocarbon structure and that does not exhibit acid decomposability. This makes it possible to reduce leaching of low-molecular-weight components, during liquid immersion exposure, from the resist film into the immersion liquid. The repeating unit that has an alicyclic hydrocarbon structure and that does not exhibit acid decomposability may be, for example, a repeating unit derived from 1-adamantyl (meth)acrylate, diamantyl (meth)acrylate, tricyclodecanyl (meth)acrylate, or cyclohexyl (meth)acrylate.

### (Repeating Unit Having Neither Hydroxy Group Nor Cyano Group and Represented by Formula (III))

The resin (A) may have a repeating unit having neither a hydroxy group nor a cyano group and represented by a formula (III).

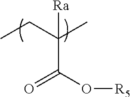

In the formula (III), R₅ represents a hydrocarbon group having at least one ring structure and having neither a hydroxy group nor a cyano group.

Ra represents a hydrogen atom, an alkyl group, or a —CH₂—O—Ra₂ group. In the formula, Raz represents a hydrogen atom, an alkyl group, or an acyl group.

Examples of the repeating unit having neither a hydroxy group nor a cyano group and represented by the formula (III) include those described in paragraphs [0087] to [0094] of JP2014-098921A.

### (Other Repeating Units)

Furthermore, the resin (A) may have another repeating unit other than the above-described repeating units.

For example, the resin (A) may have a repeating unit selected from the group consisting of a repeating unit having an oxathiane ring group, a repeating unit having an oxazolone ring group, a repeating unit having a dioxane ring group, and a repeating unit having a hydantoin ring group.

The resin (A) may have, in addition to the above-described repeating units, various repeating units for the purpose of adjusting dry etching resistance, suitability for a standard developer, adhesiveness to a substrate, resist profile, resolution, heat resistance, sensitivity, and the like.

For the resin (A), particularly in the case where the composition of the present invention is used as an actinic ray-sensitive or radiation-sensitive resin composition for ArF, all the repeating units are preferably constituted by repeating units derived from compounds having ethylenically unsaturated bonds. In particular, all the repeating units are also preferably constituted by (meth)acrylate-based repeating units. When all the repeating units are constituted by (meth)acrylate-based repeating units, all the repeating units may be methacrylate-based repeating units, all the repeating units may be acrylate-based repeating units, or all the repeating units may each be a methacrylate-based repeating unit or an acrylate-based repeating unit. The acrylate-based repeating units preferably account for 50 mol % or less of all the repeating units.

The resin (A) can be synthesized by an ordinary method (for example, radical polymerization).

The resin (A) preferably has a weight-average molecular weight (Mw) of 30,000 or less, more preferably 1,000 to 30,000, still more preferably 3,000 to 30,000, particularly preferably 5,000 to 15,000 in terms of polystyrene as determined by the GPC method.

The dispersity (molecular weight distribution, Mw/Mn) of the resin (A) is preferably 1 to 5, more preferably 1 to 3, still more preferably 1.2 to 3.0, particularly preferably 1.2 to 2.0. The smaller the dispersity, the better the resolution and the resist profile, the smoother the sidewalls of the resist pattern, and the better the roughness performance.

The content of the resin (A) in the composition of the present invention is preferably 30.0 to 99.9 mass %, more preferably 40.0 to 99.9 mass %, still more preferably 60.0 to 90.0 mass % relative to the total solid contents of the composition of the present invention.

One of such resins (A) may be used alone, or two or more of such resins (A) may be used.

When two or more resins (A) are used, the total content thereof is preferably within the above preferred content ranges.

[Compound (B) that Generates Acid Upon Irradiation with Actinic Rays or Radiation]

The composition of the present invention may further include a compound (B) which is a compound that generates an acid upon irradiation with actinic rays or radiation and is different from the compound (N).

The compound (B) may be in the form of a low-molecular-weight compound, or may be in the form of being incorporated into a part of a polymer. Alternatively, the form of a low-molecular-weight compound and the form of being incorporated into a part of a polymer may be used in combination.

When the compound (B) is in the form of a low-molecular-weight compound, the molecular weight of the compound (B) is preferably 5,000 or less, more preferably 4,000 or less, still more preferably 3,000 or less. The lower limit is not particularly limited, but is preferably 100 or more.

When the compound (B) is in the form of being incorporated into a part of a polymer, the compound (B) may be incorporated into a part of the resin (A) or incorporated into a resin different from the resin (A).

The compound (B) is preferably in the form of a low-molecular-weight compound.

The compound (B) is, for example, a compound (onium salt) represented by “M⁺ X⁻” and is preferably a compound that generates an organic acid upon exposure.

Examples of the organic acid include sulfonic acids (such as aliphatic sulfonic acids, aromatic sulfonic acids, and camphorsulfonic acid), carboxylic acids (such as aliphatic carboxylic acids, aromatic carboxylic acids, and aralkyl carboxylic acids), carbonylsulfonylimidic acid, bis(alkylsulfonyl) imidic acids, and tris(alkylsulfonyl) methide acids.

In the compound represented by “M⁺ X⁻”, M⁺ represents a cation, and preferably represents an organic cation.

The description, specific examples, and preferred ranges of M⁺ are the same as those of M⁺ in the formula (a-1) above.

In the compound represented by “M⁺ X⁻”, X⁻ represents an anion, and preferably represents an organic anion.

The organic anion is not particularly limited, and may be a mono-, di-, or higher valent organic anion.

The organic anion is preferably an anion having an extremely low ability to cause a nucleophilic reaction, more preferably a non-nucleophilic anion.

Examples of the non-nucleophilic anion include sulfonate anions (such as aliphatic sulfonate anions, aromatic sulfonate anions, and a camphorsulfonate anion), carboxylate anions (such as aliphatic carboxylate anions, aromatic carboxylate anions, and aralkyl carboxylate anions), sulfonylimide anions, bis(alkylsulfonyl)imide anions, and tris(alkylsulfonyl) methide anions.

The aliphatic moieties in the aliphatic sulfonate anions and the aliphatic carboxylate anions may each be a linear or branched alkyl group or a cycloalkyl group, and are each preferably a linear or branched alkyl group having 1 to 30 carbon atoms, or a cycloalkyl group having 3 to 30 carbon atoms.

The above alkyl group may be, for example, a fluoroalkyl group (that may have a substituent other than a fluorine atom or may be a perfluoroalkyl group).

The aryl groups in the aromatic sulfonate anions and the aromatic carboxylate anions are each preferably an aryl group having 6 to 14 carbon atoms, and examples thereof include a phenyl group, a tolyl group, and a naphthyl group.

The above-mentioned alkyl group, cycloalkyl group, and aryl group may have a substituent. Examples of the substituent include, but are not particularly limited to, a nitro group, halogen atoms such as a fluorine atom and a chlorine atom, a carboxyl group, a hydroxy group, an amino group, a cyano group, alkoxy groups (preferably having 1 to 15 carbon atoms), alkyl groups (preferably having 1 to 10 carbon atoms), cycloalkyl groups (preferably having 3 to 15 carbon atoms), aryl groups (preferably having 6 to 14 carbon atoms), alkoxycarbonyl groups (preferably having 2 to 7 carbon atoms), acyl groups (preferably having 2 to 12 carbon atoms), alkoxycarbonyloxy groups (preferably having 2 to 7 carbon atoms), alkylthio groups (preferably having 1 to 15 carbon atoms), alkylsulfonyl groups (preferably having 1 to 15 carbon atoms), alkyliminosulfonyl groups (preferably having 1 to 15 carbon atoms), and aryloxysulfonyl groups (preferably having 6 to 20 carbon atoms).

The aralkyl groups in the aralkyl carboxylate anions are each preferably an aralkyl group having 7 to 14 carbon atoms.

Examples of the aralkyl group having 7 to 14 carbon atoms include a benzyl group, a phenethyl group, a naphthylmethyl group, a naphthylethyl group, and a naphthylbutyl group. An example of the sulfonylimide anions is a saccharin anion.

The alkyl groups in the bis(alkylsulfonyl)imide anions and the tris(alkylsulfonyl) methide anions are preferably alkyl groups having 1 to 5 carbon atoms. A substituent of such an alkyl group may be a halogen atom, an alkyl group substituted with a halogen atom, an alkoxy group, an alkylthio group, an alkyloxysulfonyl group, an aryloxysulfonyl group, or a cycloalkylaryloxysulfonyl group, and is preferably a fluorine atom or an alkyl group substituted with a fluorine atom.

The alkyl groups in such a bis(alkylsulfonyl)imide anion may be bonded together to form a ring structure. This increases the acid strength.

Other examples of the non-nucleophilic anion include fluorinated phosphorus (for example, PF₆⁻), fluorinated boron (for example, BF₄⁻), and fluorinated antimony (for example, SbF₆⁻).

As the non-nucleophilic anion, an anion represented by a formula (AN1) below is also preferred.

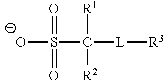

In the formula (AN1), R¹ and R² each independently represent a hydrogen atom or a substituent.

The substituent is not particularly limited, but is preferably a group that is not an electron-withdrawing group. Examples of the group that is not an electron-withdrawing group include hydrocarbon groups, a hydroxy group, oxyhydrocarbon groups, oxycarbonyl hydrocarbon groups, an amino group, hydrocarbon-substituted amino groups, and hydrocarbon-substituted amide groups.

Each group that is not an electron-withdrawing group is preferably independently −R′, −OH, —OR′, —OCOR′, —NH₂, —NR′₂, —NHR′, or —NHCOR′. R′ is a monovalent hydrocarbon group.

Examples of the monovalent hydrocarbon group represented by R′include monovalent linear or branched hydrocarbon groups such as alkyl groups, e.g., a methyl group, an ethyl group, a propyl group, and a butyl group, alkenyl groups, e.g., an ethenyl group, a propenyl group, and a butenyl group, and alkynyl groups, e.g., an ethynyl group, a propynyl group, and a butynyl group; monovalent alicyclic hydrocarbon groups such as cycloalkyl groups, e.g., a cyclopropyl group, a cyclobutyl group, a cyclopentyl group, a cyclohexyl group, a norbornyl group, and an adamantyl group, and cycloalkenyl groups, e.g., a cyclopropenyl group, a cyclobutenyl group, a cyclopentenyl group, and a norbornenyl group; and monovalent aromatic hydrocarbon groups such as aryl groups, e.g., a phenyl group, a tolyl group, a xylyl group, a mesityl group, a naphthyl group, a methylnaphthyl group, an anthryl group, and a methylanthryl group, and aralkyl groups, e.g., a benzyl group, a phenethyl group, a phenylpropyl group, a naphthylmethyl group, and an anthrylmethyl group.

Of these, R¹ and R² are each independently preferably a hydrocarbon group (preferably a cycloalkyl group) or a hydrogen atom.

L represents a divalent linking group.

When a plurality of L's are present, L's may be the same or different.

Examples of the divalent linking group include —O—CO—O—, —COO—, —CONH—, —CO—, —O—, —S—, —SO—, —SO₂—, alkylene groups (preferably having 1 to 6 carbon atoms), cycloalkylene groups (preferably having 3 to 15 carbon atoms), alkenylene groups (preferably having 2 to 6 carbon atoms), and divalent linking groups provided by combining a plurality of these groups. Among these, the divalent linking group is preferably —O—CO—O—, —COO—, —CONH—, —CO—, —O—, —SO₂—, —O—CO—O-alkylene group-, —COO-alkylene group-, or —CONH-alkylene group-, more preferably —O—CO—O—, —O—CO—O-alkylene group-, —COO—, —CONH—, —SO₂—, or —COO-alkylene group-.

L is preferably, for example, a group represented by a formula (AN1-1) below.

*^{a}—(CR^{2a}₂)_{X}-Q-(CR^{2b}₂)_{y}—*^{b} (AN1-1)

In the formula (AN1-1), *^{a} represents a bonding site to R³ in the formula (AN1).

*^{b} represents a bonding site to —C(R¹)(R²)— in the formula (AN1).

X and Y each independently represent an integer of 0 to 10 and preferably an integer of 0 to 3.

R^{2a} and R^{2b} each independently represent a hydrogen atom or a substituent.

When a plurality of R^{2a}'s and a plurality of R^{2b}'s are present, the plurality of R^{2a}'s present may be the same or different and the plurality of R^{2b}'s present may be the same or different.

However, when Y is 1 or more, R^{2b}'s in CR^{2b}₂ directly bonded to —C(R¹)(R²)— in the formula (AN1) are not fluorine atoms.

Q represents *^{A}—O—CO—O—*^{B}, *^{A}—CO—*^{B}, *^{A}—CO—O—*^{B}, *^{A}—O—CO—*^{B}, *^{A}—O—*^{B}, *^{A}—S—*^{B}, or *^{A}—SO₂—*^{B}.

However, when X+Y in the formula (AN1-1) is 1 or more and R^{2a}'s and R^{2b}'s in the formula (AN1-1) are all hydrogen atoms, Q represents *^{A}—O—CO—O—*^{B}, *^{A}—CO—*^{B}, *^{A}O—CO—*^{B}, *^{A}—O—*^{B}, *^{A}—S—*^{B}, or *^{A}—SO₂—*^{B}.

*^{A} represents a bonding site on the R³ side in the formula (AN1), and *^{B} represents a bonding site on the —SO₃ side in the formula (AN1).

In the formula (AN1), R³ represents an organic group.

The organic group is not particularly limited as long as it has 1 or more carbon atoms, and may be a linear group (for example, a linear alkyl group), a branched group (for example, a branched alkyl group such as a t-butyl group), or a cyclic group. The organic group may have or may not have a substituent. The organic group may have or may not have a heteroatom (such as an oxygen atom, a sulfur atom, and/or a nitrogen atom).

In particular, R³ is preferably an organic group having a ring structure. The ring structure may be monocyclic or polycyclic, and may have a substituent. The ring in the organic group including a ring structure is preferably directly bonded to L in the formula (AN1).

The organic group having a ring structure may have or may not have, for example, a heteroatom (such as an oxygen atom, a sulfur atom, and/or a nitrogen atom). One or more carbon atoms forming the ring structure may each be substituted with a heteroatom.

The organic group having a ring structure is preferably, for example, a hydrocarbon group having a ring structure, a lactone ring group, or a sultone ring group. In particular, the organic group having a ring structure is preferably a hydrocarbon group having a ring structure.

The hydrocarbon group having a ring structure is preferably a monocyclic or polycyclic cycloalkyl group. These groups may have a substituent.

The cycloalkyl group may be monocyclic (such as a cyclohexyl group) or polycyclic (such as an adamantyl group), and preferably has 5 to 12 carbon atoms.

The lactone group and sultone group are preferably, for example, groups formed by removing one hydrogen atom from a ring member atom constituting the lactone structure or the sultone structure in any of the structures represented by the formulae (LC1-1) to (LC1-22) described above and the structures represented by the formulae (SL1-1) to (SL1-3) described above.

The non-nucleophilic anion may be a benzenesulfonate anion, and is preferably a benzenesulfonate anion substituted with a branched alkyl group or a cycloalkyl group.

As the non-nucleophilic anion, an anion represented by a formula (AN2) below is also preferred.

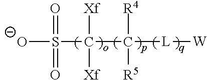

In the formula (AN2), o represents an integer of 1 to 3. p represents an integer of 0 to 10. q represents an integer of 0 to 10.

Xf's each represent a hydrogen atom, a fluorine atom, an alkyl group substituted with at least one fluorine atom, or an organic group having no fluorine atom. The number of carbon atoms of the alkyl group is preferably 1 to 10, more preferably 1 to 4. The alkyl group substituted with at least one fluorine atom is preferably a perfluoroalkyl group.

Xf's are each preferably a fluorine atom or a perfluoroalkyl group having 1 to 4 carbon atoms, more preferably a fluorine atom or CF₃. Still more preferably, both Xf's are fluorine atoms.

R⁴ and R⁵ each independently represent a hydrogen atom, a fluorine atom, an alkyl group, or an alkyl group substituted with at least one fluorine atom. When a plurality of R⁴'s and a plurality of R⁵'s are present, the plurality of R⁴'s may be the same or different and the plurality of R⁵'s may be the same or different.

The alkyl group represented by R⁴ and R⁵ preferably has 1 to 4 carbon atoms. The alkyl group may have a substituent. R⁴ and R⁵ are preferably hydrogen atoms.

L represents a divalent linking group. The definition of L is the same as that of L in the formula (AN1).

W represents an organic group including a ring structure. In particular, a cyclic organic group is preferable.

The cyclic organic group may be, for example, an alicyclic group, an aryl group, or a heterocyclic group.

The alicyclic group may be monocyclic or polycyclic. Examples of the monocyclic alicyclic group include monocyclic cycloalkyl groups such as a cyclopentyl group, a cyclohexyl group, and a cyclooctyl group. Examples of the polycyclic alicyclic group include polycyclic cycloalkyl groups such as a norbornyl group, a tricyclodecanyl group, a tetracyclodecanyl group, a tetracyclododecanyl group, and an adamantyl group. Of these, alicyclic groups having a bulky structure with 7 or more carbon atoms, such as a norbornyl group, a tricyclodecanyl group, a tetracyclodecanyl group, a tetracyclododecanyl group, and an adamantyl group, are preferred.

The aryl group may be monocyclic or polycyclic. Examples of the aryl group include a phenyl group, a naphthyl group, a phenanthryl group, and an anthryl group.

The heterocyclic group may be monocyclic or polycyclic. In particular, in the case of a polycyclic heterocyclic group, diffusion of the acid can be further suppressed. The heterocyclic group may have aromaticity or may not have aromaticity. Examples of the heterocycle having aromaticity include a furan ring, a thiophene ring, a benzofuran ring, a benzothiophene ring, a dibenzofuran ring, a dibenzothiophene ring, and a pyridine ring. Examples of the heterocycle having no aromaticity include a tetrahydropyran ring, a lactone ring, a sultone ring, and a decahydroisoquinoline ring. The heterocycle in the heterocyclic group is preferably a furan ring, a thiophene ring, a pyridine ring, or a decahydroisoquinoline ring.

The cyclic organic group may have a substituent. Examples of the substituent include alkyl groups (which may be linear or branched and preferably have 1 to 12 carbon atoms), cycloalkyl groups (which may be monocyclic, polycyclic, or spirocyclic and preferably have 3 to 20 carbon atoms), aryl groups (preferably having 6 to 14 carbon atoms), a hydroxy group, alkoxy groups, an ester group, an amide group, a urethane group, a ureido group, a thioether group, a sulfonamide group, and a sulfonic acid ester group. The carbon constituting the cyclic organic group (carbon that contributes to ring formation) may be a carbonyl carbon.

The anion represented by the formula (AN2) is preferably SO₃⁻—CF₂—CH₂—OCO-(L)_{q′}-W, SO₃⁻—CF₂—CHF—CH₂—OCO-(L)_{q′}-W, SO₃⁻—CF₂—COO-(L)_{q′}—W, SO₃⁻—CF₂—CF₂—CH₂—CH₂-(L)_{q}-W, or SO₃⁻—CF₂—CH(CF₃)—OCO-(L)_{q}-W. Here, L, q, and W are the same as those in the formula (AN2). q′ represents an integer of 0 to 10.

As the non-nucleophilic anion, an aromatic sulfonate anion represented by a formula (AN3) below is also preferred.

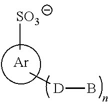

In the formula (AN3), Ar represents an aryl group (such as a phenyl group) and may further have a substituent other than the sulfonate anion and the -(D-B) group. Examples of the substituent that Ar may further have include a fluorine atom and a hydroxy group.

n represents an integer of 0 or more. n is preferably 1 to 4, more preferably 2 to 3, still more preferably 3.

D represents a single bond or a divalent linking group. Examples of the divalent linking group include an ether group, a thioether group, a carbonyl group, a sulfoxide group, a sulfone group, a sulfonic acid ester group, an ester group, and groups provided by combining two or more of these groups.

B represents a hydrocarbon group.

B is preferably an aliphatic hydrocarbon group, more preferably an isopropyl group, a cyclohexyl group, or an aryl group that may further have a substituent (such as a tricyclohexylphenyl group).

The non-nucleophilic anion is also preferably a disulfonamide anion.

The disulfonamide anion is, for example, an anion represented by N—(SO₂—R^{q})₂.

Here, each R^{q} represents an alkyl group that may have a substituent and is preferably a fluoroalkyl group, more preferably a perfluoroalkyl group. Two R″'s may be bonded together to form a ring. The group formed by bonding two Ra's together is preferably an alkylene group that may have a substituent, more preferably a fluoroalkylene group, still more preferably a perfluoroalkylene group. The alkylene group preferably has 2 to 4 carbon atoms.

The compound (B) is also preferably at least one selected from the group consisting of compounds (I) and (II).

### (Compound (I))

The compound (I) is a compound that has one or more structural moieties X described below and one or more structural moieties Y described below and that generates an acid including a first acidic moiety described below and derived from the structural moiety X and a second acidic moiety described below and derived from the structural moiety Y upon irradiation with actinic rays or radiation.

Structural moiety X: A structural moiety that is constituted by an anionic moiety A₁⁻ and a cationic moiety M₁⁺ and that forms the first acidic moiety represented by HA₁ upon irradiation with actinic rays or radiation

Structural moiety Y: A structural moiety that is constituted by an anionic moiety A₂⁻ and a cationic moiety M₂⁺ and that forms the second acidic moiety represented by HA₂ upon irradiation with actinic rays or radiation

The compound (I) satisfies the following condition I.

Condition I: A compound PI obtained by replacing the cationic moiety M₁⁺ in the structural moiety X and the cationic moiety M₂⁺ in the structural moiety Y in the compound (I) with H⁺ has an acid dissociation constant a1 derived from an acidic moiety represented by HA₁ obtained by replacing the cationic moiety M₁⁺ in the structural moiety X with H⁺ and an acid dissociation constant a2 derived from an acidic moiety represented by HA₂ obtained by replacing the cationic moiety M2⁺ in the structural moiety Y with H⁺, and the acid dissociation constant a2 is larger than the acid dissociation constant a1.

The condition I will be more specifically described below.

When the compound (I) is, for example, a compound that generates an acid having one first acidic moiety derived from the structural moiety X and one second acidic moiety derived from the structural moiety Y, the compound PI corresponds to a “compound having HA₁ and HA₂”.

The acid dissociation constant a1 and the acid dissociation constant a2 of the compound PI are more specifically described as follows. In determination of the acid dissociation constants of the compound PI, the pKa determined when the compound PI turns into a “compound having A₁⁻ and HA₂” is the acid dissociation constant a1, and the pKa determined when the “compound having A₁⁻ and HA₂” turns into a “compound having A₁⁻ and A₂⁻” is the acid dissociation constant a2.

When the compound (I) is, for example, a compound that generates an acid having two first acidic moieties derived from the structural moiety X and one second acidic moiety derived from the structural moiety Y, the compound PI corresponds to a “compound having two HA₁'s and one HA₂”.

In determination of the acid dissociation constants of the compound PI, the acid dissociation constant determined when the compound PI turns into a “compound having one A₁⁻, one HA₁, and one HA₂” and the acid dissociation constant determined when the “compound having one A₁⁻, one HA₁, and one HA₂” turns into a “compound having two A₁⁻'s and one HA₂” each correspond to the above-described acid dissociation constant a1. The acid dissociation constant determined when the “compound having two A₁⁻ 's and one HA₂” turns into a “compound having two A₁⁻'s and A₂⁻” corresponds to the acid dissociation constant a2. That is, when the compound PI has a plurality of acid dissociation constants derived from acidic moieties represented by HA₁ obtained by replacing the cationic moiety M1⁺ in the structural moiety X with H⁺, the value of the acid dissociation constant a2 is larger than the largest value among the plurality of acid dissociation constants a1. Note that, when the acid dissociation constant determined when the compound PI turns into a “compound having one A₁⁻, one HA₁, and one HA₂” is defined as aa and the acid dissociation constant determined when the “compound having one A₁⁻, one HA₁, and one HA₂” turns into a “compound having two A₁⁻ 's and one HA₂” is defined as ab, the relation between aa and ab satisfies aa<ab.

The acid dissociation constant a1 and the acid dissociation constant a2 can be determined by the above-described method of measuring an acid dissociation constant.

The compound PI corresponds to an acid generated when the compound (I) is irradiated with actinic rays or radiation.

When the compound (I) has two or more structural moieties X, the structural moieties X may be the same or different. Two or more A₁⁻'s and two or more M₁⁺'s may be individually the same or different.

In the compound (I), A₁⁻ and A₂⁻, and M₁⁺ and M₂⁺ may be individually the same or different, but A₁⁻ and A₂⁻ are preferably different.

In the compound PI, the difference (absolute value) between the acid dissociation constant a1 (in the case where a plurality of acid dissociation constants a1 are present, the maximum value thereof) and the acid dissociation constant a2 is preferably 0.1 or more, more preferably 0.5 or more, still more preferably 1.0 or more. The upper limit value of the difference (absolute value) between the acid dissociation constant a1 (in the case where a plurality of acid dissociation constants a1 are present, the maximum value thereof) and the acid dissociation constant a2 is not particularly limited, but is, for example, 16 or less.

In the compound PI, the acid dissociation constant a2 is preferably 20 or less, more preferably 15 or less. The lower limit value of the acid dissociation constant a2 is preferably −4.0 or more.

In the compound PI, the acid dissociation constant a1 is preferably 2.0 or less, more preferably 0 or less. The lower limit value of the acid dissociation constant a1 is preferably −20.0 or more.

The anionic moiety A₁⁻ and the anionic moiety A₂⁻ are structural moieties including a negatively charged atom or atomic group and are, for example, structural moieties selected from the group consisting of formulae (AA-1) to (AA-3) and formulae (BB-1) to (BB-6) below.

The anionic moiety A₁⁻ is preferably one that can form an acidic moiety having a small acid dissociation constant, in particular, more preferably any one of the formulae (AA-1) to (AA-3), still more preferably any one of the formulae (AA-1) and (AA-3).

The anionic moiety A₂⁻ is preferably one that can form an acidic moiety having a larger acid dissociation constant than the anionic moiety A₁⁻, more preferably any one of the formulae (BB-1) to (BB-6), still more preferably any one of the formulae (BB-1) and (BB-4).

In the formulae (AA-1) to (AA-3) and the formulae (BB-1) to (BB-6) below, * represents a bonding site.

In the formula (AA-2), each RA represents a monovalent organic group. The monovalent organic group represented by RA is not particularly limited and may be, for example, a cyano group, a trifluoromethyl group, or a methanesulfonyl group.

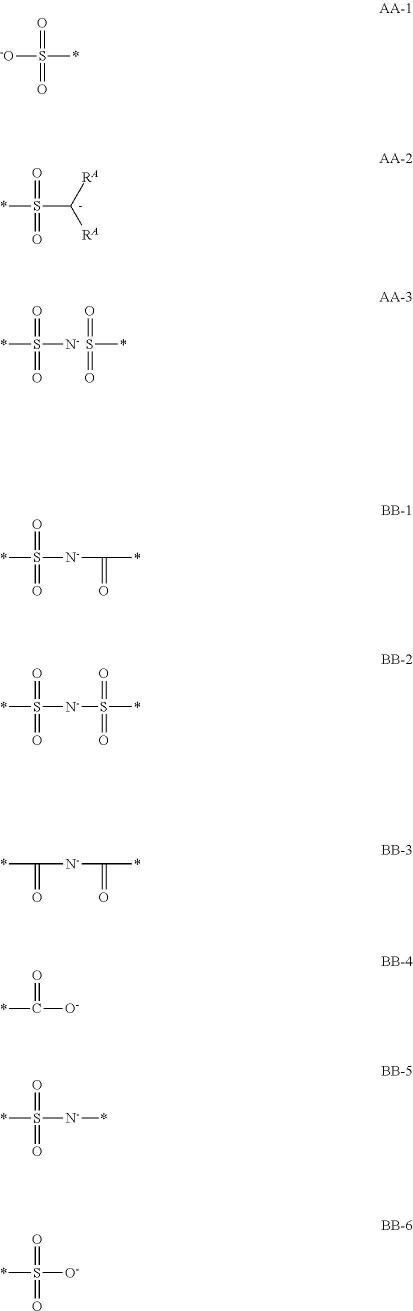

The cationic moiety M1+ and the cationic moiety M2+ are structural moieties including a positively charged atom or atomic group and are, for example, singly charged organic cations. Examples of organic cations include the above-described organic cations represented by M⁺.

### (Compound (II))

The compound (II) is a compound that has two or more structural moieties X described above and one or more structural moieties Z described below and that generates an acid including two or more first acidic moieties derived from the structural moieties X and the structural moiety Z upon irradiation with actinic rays or radiation.

Structural moiety Z: A nonionic moiety capable of neutralizing an acid

In the compound (II), the definition of the structural moiety X, and the definitions of A₁⁻ and M₁⁺ are the same as the definition of the structural moiety X, and the definitions of A₁⁻ and M₁⁺, respectively, in the compound (I) described above, and preferred embodiments are also the same as those of the structural moiety X, A₁⁻, and M₁⁺ in the compound (I).

In a compound PII obtained by replacing each of the cationic moieties M₁⁺ in the structural moieties X in the compound (II) with H⁺, a preferred range of the acid dissociation constant a1 derived from the acidic moiety represented by HA₁ obtained by replacing the cationic moiety M1⁺ in one of the structural moieties X by H⁺ is the same as that of the acid dissociation constant a1 in the compound PI.

When the compound (II) is, for example, a compound that generates an acid having two first acidic moieties derived from the structural moieties X and the structural moiety Z, the compound PII corresponds to a “compound having two HA₁'s”. In determination of the acid dissociation constants of this compound PII, the acid dissociation constant determined when the compound PII turns into a “compound having one A₁⁻ and one HA₁” and the acid dissociation constant determined when the “compound having one A₁⁻ and one HA₁” turns into a “compound having two A₁⁻'s” each correspond to the acid dissociation constant a1.

The acid dissociation constant a1 can be determined by the above-described method of measuring an acid dissociation constant.

The compound PII corresponds to an acid generated when the compound (II) is irradiated with actinic rays or radiation.

The two or more structural moieties X may be the same or different. Two or more A₁⁻'s and two or more M₁⁺'s may be individually the same or different.

The nonionic moiety capable of neutralizing an acid in the structural moiety Z is not particularly limited, and is preferably, for example, a moiety including a functional group having an electron or a group that can electrostatically interact with a proton.

Examples of the functional group having an electron or a group that can electrostatically interact with a proton include a functional group having a macrocyclic structure such as a cyclic polyether, and a functional group having a nitrogen atom having an unshared electron pair that does not contribute to x-conjugation. The nitrogen atom having an unshared electron pair that does not contribute to x-conjugation is, for example, a nitrogen atom having a partial structure represented by any of the following formulae.

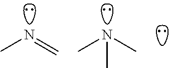

Unshared electron pair

Examples of the partial structure of the functional group having an electron or the group that can electrostatically interact with a proton include crown ether structures, azacrown ether structures, primary to tertiary amine structures, a pyridine structure, an imidazole structure, and a pyrazine structure. Among these, primary to tertiary amine structures are preferable.

Examples of moieties that are not cations and that the compound (I) and the compound (II) may have are shown below.

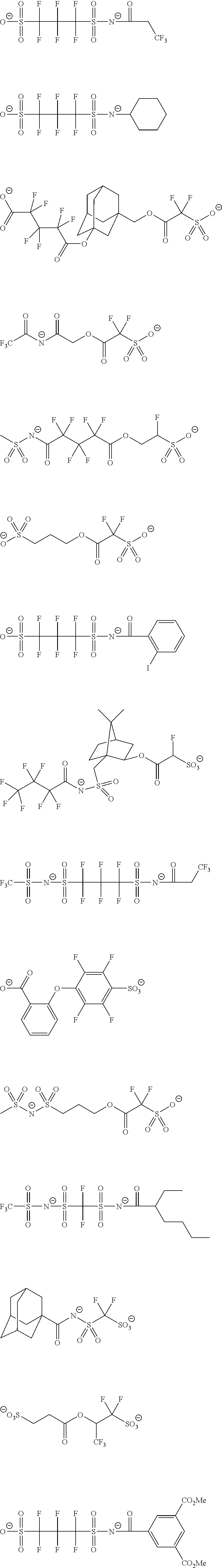

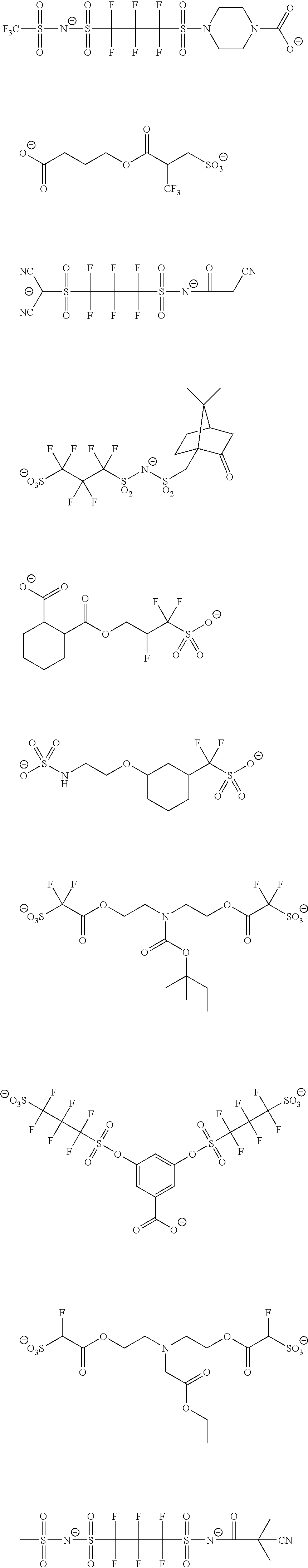

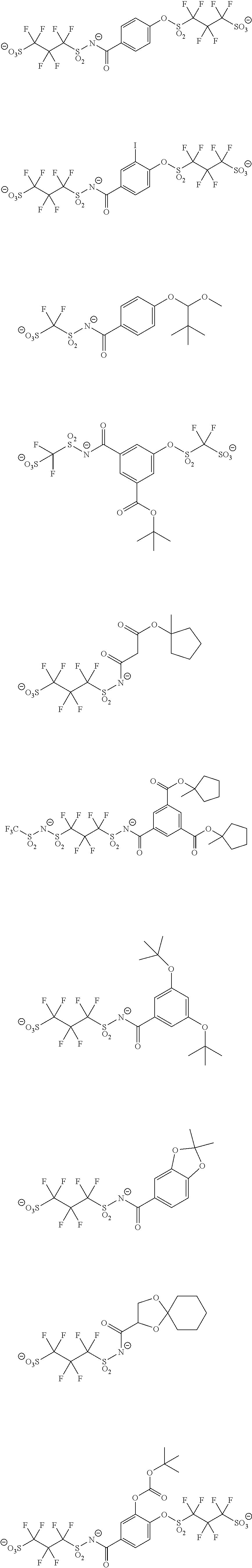

Specific examples of the compound (B) include the compounds described in paragraphs [0320] to [0321] of WO2022/172715A. The above description is incorporated herein.

The composition of the present invention may or may not include the compound (B). When the composition of the present invention includes the compound (B), the content of the compound (B) is not particularly limited, but is preferably 0.1 mass % or more, more preferably 1 mass % or more relative to the total solid contents of the composition of the present invention. When the composition of the present invention includes the compound (B), the content of the compound (B) is preferably 30 mass % or less, more preferably 20 mass % or less relative to the total solid contents of the composition of the present invention.

Such compounds (B) may be used alone or in combination of two or more thereof. When two or more compounds (B) are used, the total content thereof is preferably within the above preferred content ranges.

### [Acid Diffusion Control Agent]

The composition of the present invention may further include an acid diffusion control agent (also referred to as a “compound (C)”).

The compound (C) is preferably a compound different from the compound (N), the resin (A), and the compound (B).

The acid diffusion control agent can act as a quencher that traps an acid generated from the photoacid generator or the like upon exposure to suppress a reaction of the resin whose polarity is increased by the action of an acid in unexposed portions, the reaction being caused by an excess of generated acid.

The type of compound (C) is not particularly limited, and examples thereof include a basic compound (CA), a low-molecular-weight compound (CB) having a nitrogen atom and having a group that leaves due to the action of an acid, and a compound (CC) that undergoes a reduction or loss of the acid diffusion control ability upon irradiation with actinic rays or radiation.

Examples of the compound (CC) include an onium salt compound (CD) that becomes a weak acid relative to the photoacid generator, and a basic compound (CE) that undergoes a reduction or loss of the basicity upon irradiation with actinic rays or radiation.

Specific examples of the basic compound (CA) include those described in paragraphs [0132] to [0136] of WO2020/066824A. Specific examples of the basic compound (CE) that undergoes a reduction or loss of the basicity upon irradiation with actinic rays or radiation include those described in paragraphs [0137] to [0155] of WO2020/066824A and those described in paragraph [0164] of WO2020/066824A. Specific examples of the low-molecular-weight compound (CB) having a nitrogen atom and having a group that leaves due to the action of an acid include those described in paragraphs [0156] to [0163] of WO2020/066824A.

Specific examples of the onium salt compound (CD) that becomes a weak acid relative to the photoacid generator include those described in paragraphs [0305] to [0314] of WO2020/158337A.

In addition to the above, publicly known compounds disclosed in, for example, paragraphs [0627] to [0664] of US2016/0070167A, paragraphs [0095] to [0187] of US2015/0004544A, paragraphs [0403] to [0423] of US2016/0237190A, and paragraphs [0259] to [0328] of US2016/0274458A can be suitably used as the acid diffusion control agent.

The composition of the present invention may or may not include the compound (C). When the composition of the present invention includes the compound (C), the content of the compound (C) is preferably 0.01 to 30 mass %, more preferably 0.05 to 20 mass %, still more preferably 0.1 to 15 mass % relative to the total solid contents of the composition of the present invention.

Such compounds (C) may be used alone or in combination of two or more thereof. When two or more compounds (C) are used, the total content thereof is preferably within the above preferred content ranges.

### [Hydrophobic Resin]

The composition of the present invention may further include a hydrophobic resin (also referred to as a “hydrophobic resin”) different from the resin (A).

The hydrophobic resin is preferably designed so as to be localized in the surface of a resist film. However, unlike surfactants, the hydrophobic resin does not necessarily need to have a hydrophilic group in the molecule, and does not necessarily contribute to homogeneous mixing of a polar substance and a nonpolar substance.

Advantages due to the addition of the hydrophobic resin may be control of static and dynamic contact angles at the surface of the resist film for water, and suppression of outgassing.

From the viewpoint of localization in the surface layer of the film, the hydrophobic resin preferably has one or more, more preferably two or more selected from the group consisting of a fluorine atom, a silicon atom, and a CH₃ partial structure included in a side chain moiety of the resin. The hydrophobic resin preferably has a hydrocarbon group having 5 or more carbon atoms. The resin may have such a group in the main chain thereof or, as a substituent, in a side chain thereof.

Examples of the hydrophobic resin include the compounds described in paragraphs [0275] to [0279] of WO2020/004306A.

The composition of the present invention may or may not include the hydrophobic resin. When the composition of the present invention includes a hydrophobic resin, the content of the hydrophobic resin is preferably 0.01 to 20 mass %, more preferably 0.1 to 15 mass % relative to the total solid contents of the composition of the present invention.

Such hydrophobic resins may be used alone or in combination of two or more thereof. When two or more hydrophobic resins are used, the total content thereof is preferably within the above preferred content ranges.

### [Surfactant]

The composition of the present invention may include a surfactant. The composition including a surfactant enables the formation of a pattern having higher adhesiveness and less development defects.

The surfactant is preferably a fluorine-based and/or silicon-based surfactant.

Examples of the fluorine-based and/or silicon-based surfactant include the surfactants disclosed in paragraphs and of WO2018/193954A.

Such surfactants may be used alone or in combination of two or more thereof.

The composition of the present invention may or may not include the surfactant. When the composition of the present invention includes a surfactant, the content of the surfactant is preferably 0.0001 to 2.0 mass %, more preferably 0.0005 to 1.0 mass %, still more preferably 0.1 to 1.0 mass % relative to the total solid contents of the composition of the present invention.

Such surfactants may be used alone or in combination of two or more thereof. When two or more surfactants are used, the total content thereof is preferably within the above preferred content ranges.

### [Solvent]

The composition of the present invention preferably includes a solvent.

The solvent preferably includes at least one of (M1) a propylene glycol monoalkyl ether carboxylate or (M2) at least one selected from the group consisting of propylene glycol monoalkyl ethers, lactates, acetates, alkoxypropionates, chain ketones, cyclic ketones, lactones, and alkylene carbonates. The solvent may further include a component other than the components (M1) and (M2).

It is preferable to combine the above-described solvent and the above-described resin from the viewpoints of improving coatability of the composition of the present invention and reducing the number of development defects in a pattern. Since the above-described solvent is well-balanced in terms of solubility of the resin described above, boiling point, and viscosity, for example, unevenness of the film thickness of the resist film can be reduced, and the generation of precipitates during spin coating can be suppressed.

Details of the component (M1) and the component (M2) are described in paragraphs [0218] to [0226] of WO2020/004306A, the contents of which are incorporated herein by reference.

When the solvent further includes a component other than the components (M1) and (M2), the content of the component other than the components (M1) and (M2) is preferably 5 to 30 mass % relative to the total amount of the solvent.

The content of the solvent in the composition of the present invention is determined such that the concentration of solid contents is preferably 0.5 to 30 mass %, more preferably 1 to 20 mass %. This further improves the coatability of the composition of the present invention. [Other additives]

The composition of the present invention may further include a dissolution inhibiting compound, a dye, a plasticizer, a photosensitizer, a light absorbent, and/or a compound that improves solubility in developers (for example, a phenolic compound having a molecular weight of 1,000 or less or an alicyclic or aliphatic compound including a carboxyl group).

The “dissolution inhibiting compound” refers to a compound that has a molecular weight of 3,000 or less and that is decomposed by the action of an acid to undergo a decrease in the degree of solubility in organic-based developers.

The content of fluorine atoms included in the total solid contents of the composition of the present invention is preferably 1 mass % or less, more preferably 0.5 mass % or less, still more preferably 0.1 mass % or less.

The composition of the present invention is also suitably used as a photosensitive composition for EUV exposure.

### <Actinic Ray-Sensitive or Radiation-Sensitive Film and Pattern Forming Method>

The present invention also relates to an actinic ray-sensitive or radiation-sensitive film formed using the composition of the present invention. The actinic ray-sensitive or radiation-sensitive film of the present invention is preferably a resist film.

The procedure of a pattern forming method using the composition of the present invention is not particularly limited, but the pattern forming method preferably has steps below.
Step 1: A step of forming a resist film on a substrate using a composition of the present invention
Step 2: A step of exposing the resist film
Step 3: A step of developing the exposed resist film using a developer

The procedure of each of the steps will be described in detail below.

### <Step 1: Resist Film-Forming Step>

The step 1 is a step of forming a resist film on a substrate using a composition of the present invention.

The method of forming a resist film on a substrate using the composition of the present invention may be, for example, a method of applying the composition of the present invention to a substrate.

If necessary, the composition of the present invention is preferably filtered through a filter before the application. The filter preferably has a pore size of 0.1 μm or less, more preferably 0.05 μm or less, still more preferably 0.03 μm or less. The filter is preferably formed of polytetrafluoroethylene, polyethylene, or nylon.

The composition of the present invention can be applied to a substrate (e.g., silicon, or silicon covered with silicon dioxide) as used in the production of integrated circuit elements by an appropriate coating method using a spinner, a coater, or the like. The coating method is preferably spin-coating using a spinner. The rotational speed during spin coating using a spinner is preferably 1,000 to 3,000 rpm (rotations per minute).

After application of the composition of the present invention, the substrate may be dried to form a resist film. Note that, as needed, an underlying film (an inorganic film, an organic film, or an antireflection film) may be formed as an underlayer of the resist film.

The drying method may be, for example, a method of performing drying by heating. The heating can be performed using means included in an ordinary exposure apparatus and/or an ordinary developing apparatus, or may be performed using a hot plate or the like. The heating temperature is preferably 80 °C to 150 °C, more preferably 80 °C to 140 °C, still more preferably 80 °C to 130 °C. The heating time is preferably 30 to 1,000 seconds, more preferably 60 to 800 seconds, still more preferably 60 to 600 seconds.

The film thickness of the resist film is not particularly limited, but is preferably 10 to 120 nm from the viewpoint that a fine pattern with higher accuracy can be formed. In particular, in the case of EUV exposure, the film thickness of the resist film is more preferably 10 to 65 nm, still more preferably 15 to 50 nm. In the case of ArF liquid immersion exposure, the film thickness of the resist film is more preferably 10 to 120 nm, still more preferably 15 to 90 nm.

A topcoat may be formed, as an overlying layer of the resist film, using a topcoat composition.

Preferably, the topcoat composition does not mix with the resist film and can be uniformly applied as an overlying layer of the resist film. The topcoat is not particularly limited, a publicly known topcoat can be formed by a publicly known method, and, for example, a topcoat can be formed on the basis of the description of paragraphs [0072] to [0082] of JP2014-059543A.

For example, a topcoat including a basic compound and described in JP2013-61648A is preferably formed on the resist film. Specific examples of the basic compound that can be included in the topcoat include the basic compounds that may be included in the composition of the present invention.

The topcoat also preferably includes a compound including at least one group or bond selected from the group consisting of an ether bond, a thioether bond, a hydroxy group, a thiol group, a carbonyl bond, and an ester bond.

### (Step 2: Exposure Step)

The step 2 is a step of exposing the resist film.

The exposure method may be a method of irradiating the formed resist film with actinic rays or radiation through a predetermined mask.

Examples of the actinic rays or radiation include infrared rays, visible rays, ultraviolet rays, far-ultraviolet rays, extreme ultraviolet rays, X-rays, and electron beams. A wavelength of 250 nm or less is preferred, a wavelength of 220 nm or less is more preferred, and far-ultraviolet rays having a wavelength of 1 to 200 nm are particularly preferred. Specific examples thereof include KrF excimer laser (248 nm), ArF excimer laser (193 nm), F2 excimer laser (157 nm), EUV (13.5 nm), X-rays, and electron beams.

After the exposure and before development, baking (heating) is preferably performed. The baking accelerates the reaction in exposed portions to provide higher sensitivity and a better pattern shape.

The heating temperature is preferably 80 °C to 150 °C, more preferably 80 °C to 140 °C, still more preferably 80 °C to 130 °C.

The heating time is preferably 10 to 1,000 seconds, more preferably 10 to 180 seconds, still more preferably 30 to 120 seconds.

The heating can be performed using means included in an ordinary exposure apparatus and/or an ordinary developing apparatus, or may be performed using a hot plate or the like.

This step is also referred to as post-exposure baking.

### (Step 3: Developing Step)

The step 3 is a step of developing the exposed resist film using a developer to form a pattern.

The developer may be an alkali developer or a developer including an organic solvent (also referred to as an “organic-based developer”).

Examples of the developing method include a method of immersing the substrate for a predetermined time in a tank filled with a developer (dipping method), a method of puddling a developer over a surface of the substrate using surface tension and allowing the developer to stand for a predetermined time to perform development (puddling method), a method of spraying a developer onto a surface of the substrate (spraying method), and a method of continuously ejecting a developer over the substrate rotating at a constant rate, while scanning a developer ejection nozzle at a constant rate (dynamic dispensing method).

After the step of performing development, a step of stopping the development by performing replacement with another solvent may be performed.

The development time is not particularly limited as long as the resin in unexposed portions is sufficiently dissolved within the time, and is preferably 10 to 300 seconds, more preferably 20 to 120 seconds.

The temperature of the developer is preferably 0 °C to 50 °C, more preferably 15 °C to 35 °C.

The alkali developer used is preferably an alkaline aqueous solution including an alkali. The type of the alkaline aqueous solution is not particularly limited, but may be, for example, an alkaline aqueous solution including a quaternary ammonium salt typified by tetramethylammonium hydroxide, an inorganic alkali, a primary amine, a secondary amine, a tertiary amine, an alcoholamine, a cyclic amine, or the like. In particular, the alkali developer is preferably an aqueous solution of a quaternary ammonium salt typified by tetramethylammonium hydroxide (TMAH). An appropriate amount of an alcohol, a surfactant, or the like may be added to the alkali developer. Typically, the alkali developer preferably has an alkali concentration of 0.1 to 20 mass %. Typically, the alkali developer preferably has a pH of 10.0 to 15.0.

The organic-based developer is preferably a developer including at least one organic solvent selected from the group consisting of ketone-based solvents, ester-based solvents, alcohol-based solvents, amide-based solvents, ether-based solvents, and hydrocarbon-based solvents.

A plurality of such solvents may be mixed together, or such a solvent may be mixed with a solvent other than those described above or water. The developer as a whole preferably has a moisture content of less than 50 mass %, more preferably less than 20 mass %, still more preferably less than 10 mass %, particularly preferably contains substantially no moisture.

In the organic-based developer, the content of the organic solvent relative to the total amount of the developer is preferably 50 mass % or more and 100 mass % or less, more preferably 80 mass % or more and 100 mass % or less, still more preferably 90 mass % or more and 100 mass % or less, particularly preferably 95 mass % or more and 100 mass % or less.

### (Other Steps)

The pattern forming method preferably includes, after the step 3, a step of performing washing with a rinsing liquid.

The rinsing liquid used in the rinsing step after the developing step using the alkali developer may be, for example, pure water. Note that an appropriate amount of surfactant may be added to the pure water.

An appropriate amount of surfactant may be added to the rinsing liquid.

The rinsing liquid used in the rinsing step after the developing step using an organic-based developer is not particularly limited as long as it does not dissolve the pattern, and may be a solution including a typical organic solvent. The rinsing liquid used is preferably a rinsing liquid including at least one organic solvent selected from the group consisting of hydrocarbon-based solvents, ketone-based solvents, ester-based solvents, alcohol-based solvents, amide-based solvents, and ether-based solvents.

Examples of the method of performing the rinsing step include, but are not particularly limited to, a method of continuously ejecting a rinsing liquid onto the substrate rotating at a constant rate (spin-coating method), a method of immersing the substrate for a predetermined time in a tank filled with a rinsing liquid (dipping method), and a method of spraying a rinsing liquid onto a surface of the substrate (spraying method).

The pattern forming method may include a heating step (Post Bake) after the rinsing step. In this step, the developer and the rinsing liquid remaining between patterns and inside patterns are removed by baking. In addition, this step also provides an effect of annealing the resist pattern to improve the surface roughness of the pattern. The heating step after the rinsing step is performed usually at 40 °C to 250 °C (preferably 90 °C to 200 °C) for usually 10 seconds to 3 minutes (preferably 30 seconds to 120 seconds).

The formed pattern may be used as a mask to perform etching treatment of the substrate. Specifically, the pattern formed in the step 3 may be used as a mask to process the substrate (or the underlayer film and the substrate), thereby forming a pattern in the substrate.

The method of processing the substrate (or the underlayer film and the substrate) is not particularly limited, but is preferably a method of subjecting the substrate (or the underlayer film and the substrate) to dry etching using the pattern formed in the step 3 as a mask, thereby forming a pattern in the substrate. The dry etching is preferably oxygen plasma etching.

It is preferable that various materials used in the composition and the pattern forming method of the present invention (for example, a solvent, a developer, a rinsing liquid, an antireflection film-forming composition, a topcoat-forming composition, and the like) do not include impurities such as metals. The content of impurities included in such materials is preferably 1 mass ppm (parts per million) or less, more preferably 10 mass ppb (parts per billion) or less, still more preferably 100 mass ppt or less, particularly preferably 10 mass ppt or less, most preferably 1 mass ppt or less. The lower limit is not particularly limited, and is preferably 0 mass ppt or more. Examples of the metal impurities include Na, K, Ca, Fe, Cu, Mg, Al, Li, Cr, Ni, Sn, Ag, As, Au, Ba, Cd, Co, Pb, Ti, V, W, and Zn.

The method of removing impurities, such as metals, from the various materials may be, for example, filtration using a filter. Details of filtration using a filter are described in paragraph [0321] of WO2020/004306A.

Examples of a method for reducing the amount of impurities such as metals included in the various materials include a method of selecting, as raw materials constituting the various materials, raw materials having low metal contents, a method of subjecting raw materials constituting the various materials to filter filtration, and a method of performing distillation under conditions where contamination is reduced as much as possible by, for example, lining the inside of apparatuses with Teflon (registered trademark).

Instead of filter filtration, an adsorbing material may be used to remove impurities; alternatively, filter filtration and an adsorbing material may be used in combination. Publicly known adsorbing materials can be used as such adsorbing materials, and, for example, inorganic adsorbing materials such as silica gel and zeolite, and organic adsorbing materials such as activated carbon can be used. In order to reduce the amount of impurities such as metals included in the various materials, mixing of metal impurities in the production process needs to be prevented. Whether or not metal impurities are sufficiently removed from the production apparatuses can be checked by measuring the content of metal components included in a washing liquid that has been used for washing the production apparatuses. The content of metal components included in the washing liquid that has been used is preferably 100 mass ppt (parts per trillion) or less, more preferably 10 mass ppt or less, still more preferably 1 mass ppt or less. The lower limit is not particularly limited, and is preferably 0 mass ppt or more.

A conductive compound may be added to an organic treatment liquid such as a rinsing liquid in order to prevent failure of a chemical liquid pipe and various parts (such as a filter, an O-ring, and a tube) caused by electrostatic charging and subsequent electrostatic discharging. The conductive compound is not particularly limited, but may be, for example, methanol. The amount of addition is not particularly limited, but is preferably 10 mass % or less, more preferably 5 mass % or less from the viewpoint of maintaining preferred developing performance or rinsing performance. The lower limit is not particularly limited, and is preferably 0.01 mass % or more.

Examples of the chemical liquid pipe that can be used include various pipes made of SUS (stainless steel) or a material coated with polyethylene, polypropylene, or a fluororesin (such as polytetrafluoroethylene or a perfluoroalkoxy resin) subjected to antistatic treatment. Similarly, for the filter and the O-ring, polyethylene, polypropylene, or a fluororesin (such as polytetrafluoroethylene or a perfluoroalkoxy resin) subjected to antistatic treatment can be used.

### <Method for Producing Electronic Device>

The present invention also relates to a method for producing an electronic device, the method including the pattern forming method described above, and an electronic device produced by the production method.

In preferred embodiments of the electronic device of the present invention, the electronic device is mounted on electric or electronic devices (such as home appliances, OA (Office Automation), media-related devices, optical devices, and communication devices).

### EXAMPLES

Hereinafter, the present invention will be described in more detail with reference to Examples. Materials, amounts used, ratios, details of processes, and procedures of processes described in the following Examples may be appropriately changed without departing from the spirit of the present invention. Accordingly, the scope of the present invention should not be construed as being limited to the following Examples.

Various components used in resist compositions of Examples and Comparative Examples will be described below.

### <Compound (N)>

(I)-1 to (I)-22 were used as the compound (N). (I)-1 to (I)-22 can function as a photoacid generator. In addition, (Z)-1 was used as a photoacid generator that was not the compound (N). For convenience, (Z)-1 is also described in the column of the compound (N) in Tables 1 and 5 below.

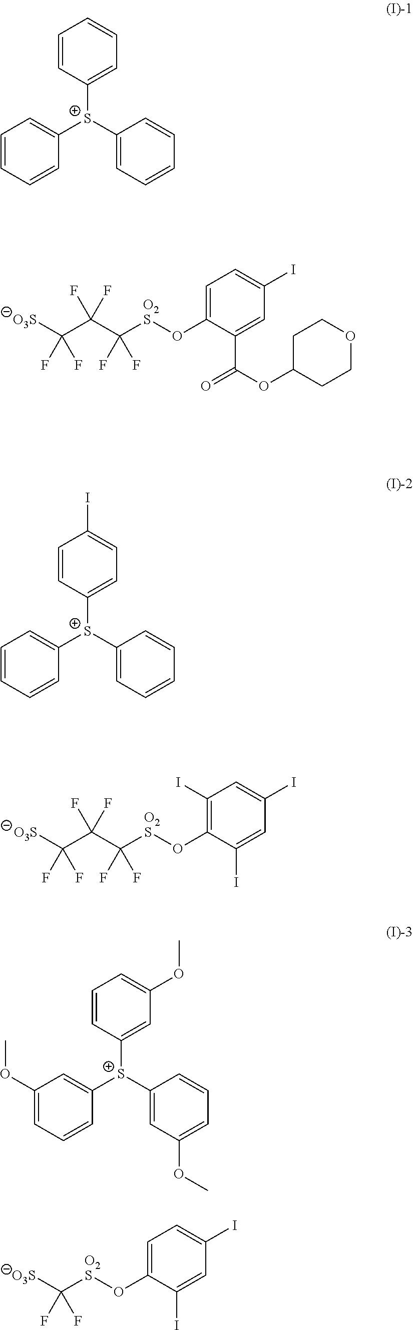

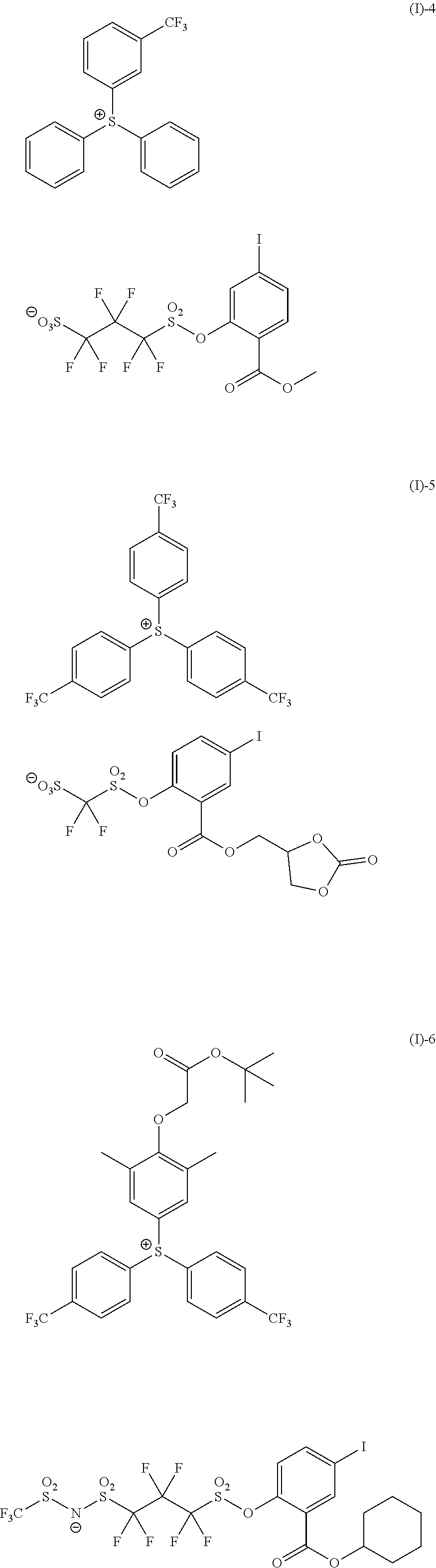

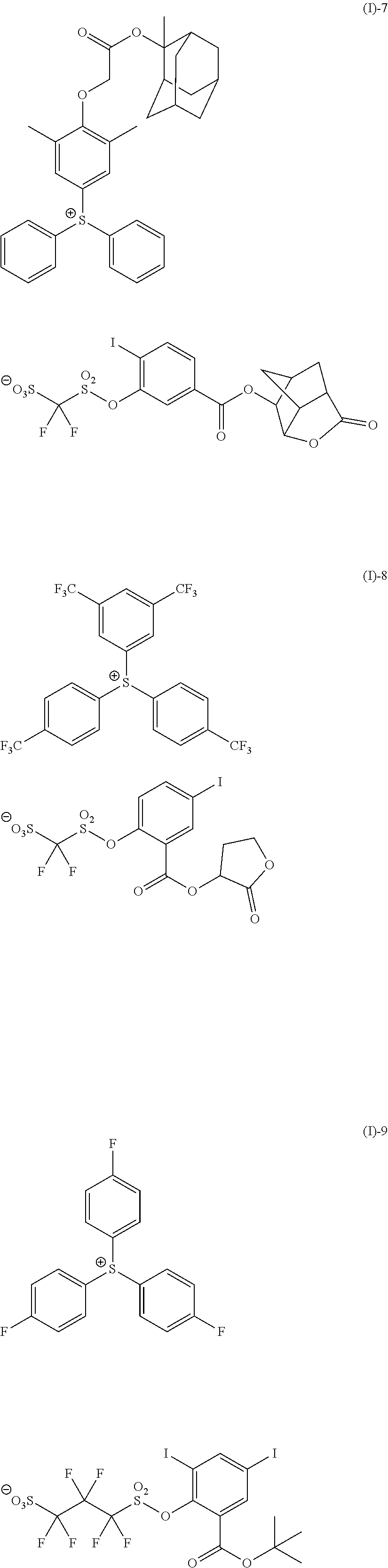

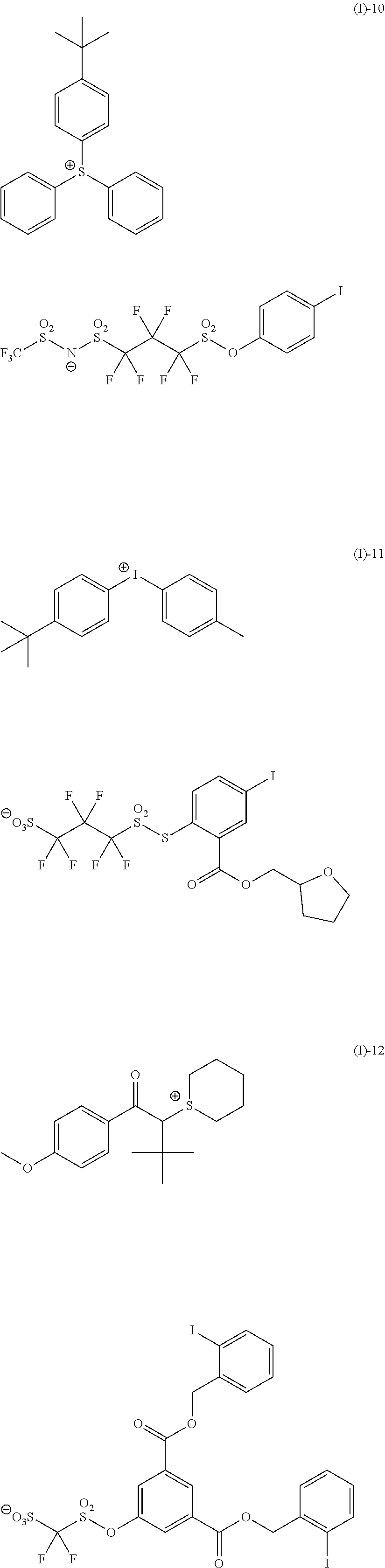

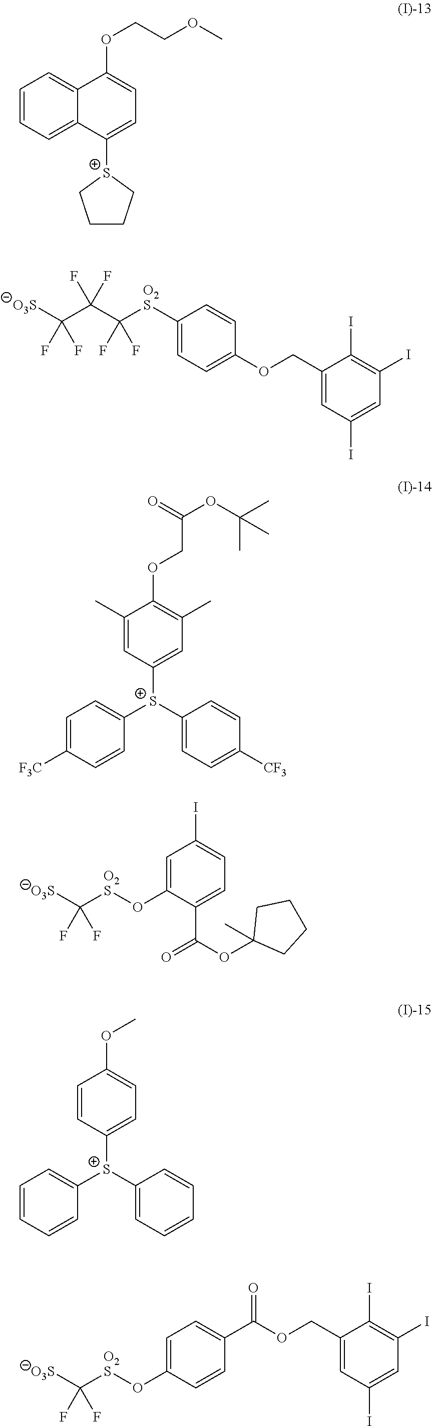

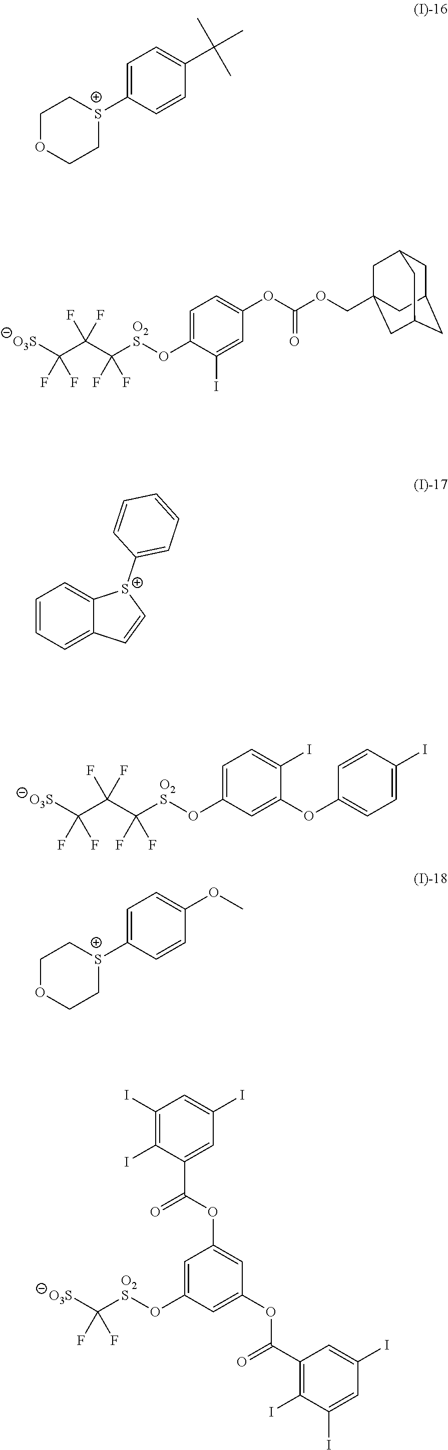

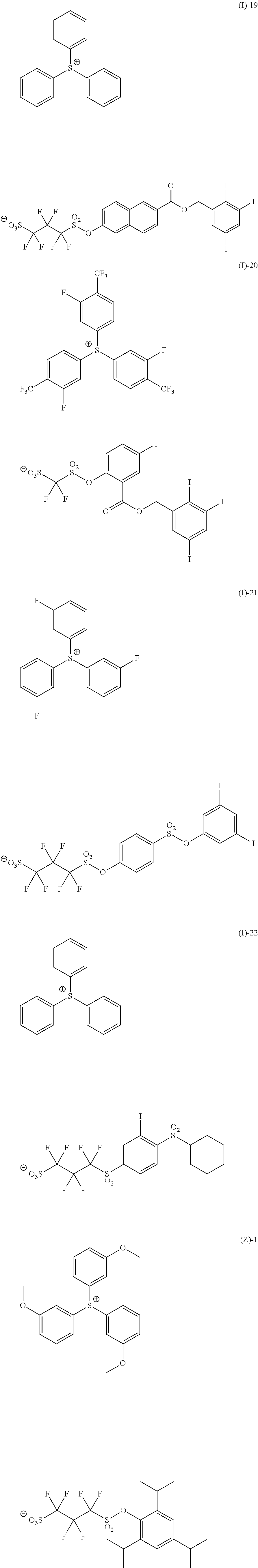

Table 1 below describes pKa of acids generated from (I)-1 to (I)-22 and (Z)-1 (generated acids) upon irradiation with actinic rays or radiation.

TABLE 1

| Compound (N) | pKa of generated acid |
|---|---|
| (I)-1 | −3.43 |
| (I)-2 | −3.44 |
| (I)-3 | −4.64 |
| (I)-4 | −3.42 |
| (I)-5 | −4.62 |
| (I)-6 | −10.73 |
| (I)-7 | −4.64 |
| (I)-8 | −4.62 |
| (I)-9 | −3.44 |
| (I)-10 | −10.72 |
| (I)-11 | −3.45 |
| (I)-12 | −4.60 |
| (I)-13 | −3.38 |
| (I)-14 | −4.59 |
| (I)-15 | −4.61 |
| (I)-16 | −3.42 |
| (I)-17 | −3.43 |
| (I)-18 | −4.67 |
| (I)-19 | −3.41 |
| (I)-20 | −4.62 |
| (I)-21 | −3.43 |
| (I)-22 | −3.44 |
| (Z)-1 | −3.40 |

A synthesis example of the compound (N) will be described below.

### (Synthesis of (I)-1)

In a three-necked flask, 120 g of 5-iodosalicylic acid (manufactured by Tokyo Chemical Industry Co., Ltd.) was dissolved in 1,350 mL of tetrahydrofuran (THF) under a nitrogen atmosphere, 73.7 g of 1,1-carbonyldiimidazole (manufactured by FUJIFILM Wako Pure Chemical Corporation) (CDI) was then added, and stirring was performed at 50 °C for 1.5 hours. To the flask, 93.0 g of tetrahydropyran-4-ol (manufactured by Tokyo Chemical Industry Co., Ltd.) was added dropwise, and the resulting reaction solution was stirred at 60 °C for five hours and cooled to room temperature (25 °C). After 1,100 mL of ethyl acetate and 1,000 mL of water were added to the reaction solution, the resulting mixture was transferred to a separatory funnel, and an organic layer was extracted. The organic layer was washed three times with 1,000 mL of ion-exchanged water, and the solvent was then distilled off to obtain 119 g of (I)-1-A as a white solid.

Under a nitrogen atmosphere in a three-necked flask, 69.5 g of (I)-1-A and the following formula (I)-1-B (129.5 g) were dissolved in 2,500 mL of THE, the resulting reaction mixture was cooled to 0 °C, and 68.4 g of cesium carbonate (manufactured by FUJIFILM Wako Pure Chemical Corporation) was then added. The temperature of the reaction mixture was increased to room temperature (25 °C), and stirring was performed for two hours to obtain a reaction liquid 1. To the reaction liquid 1, 1,200 mL of ethyl acetate was added, an organic layer was washed with 1 mol/L hydrochloric acid and water, and the solvent was then distilled off to obtain a crude product of a formula (I)-1-C below. Subsequently, the crude product of (I)-1-C was dissolved in 400 mL of THF, 400 mL of a 10 mass % aqueous sodium hydrogen carbonate solution was added to the solution, and the resulting mixture was stirred at 50 °C for three hours to obtain a reaction liquid 2. Ethyl acetate (1,000 mL) was added to the reaction liquid 2, an aqueous layer was then removed from the reaction liquid 2 with a separatory funnel, and the ethyl acetate was further distilled off to obtain a crude product of (I)-1-D. The crude product of (I)-1-D was crystallized from diisopropyl ether to obtain (I)-1-D (95 g) as a white solid.

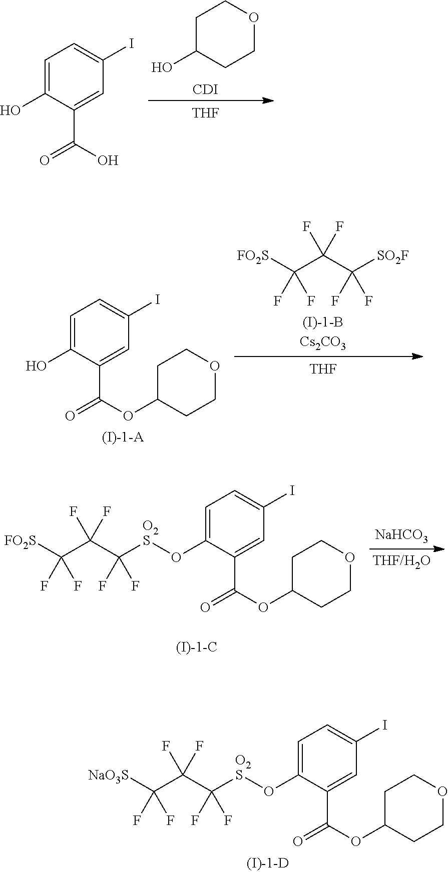

(I)-1-D (37.9 g), triphenylsulfonium bromide (manufactured by Tokyo Chemical Industry Co., Ltd.) (19.6 g), 380 mL of dichloromethane, and 500 mL of ion-exchanged water were added to a recovery flask, and the mixture was stirred at room temperature (25 °C) for 30 minutes to obtain a reaction liquid 3. An aqueous layer was removed from the reaction liquid 3 with a separatory funnel, and an organic layer was then washed twice with 350 mL of ion-exchanged water. The solvent was distilled off from the washed reaction liquid 3 under reduced pressure, and crystallization was then performed using diisopropyl ether to obtain 41 g of (I)-1 as a white solid. The obtained (I)-1 was identified by 1H-NMR (nuclear magnetic resonance).

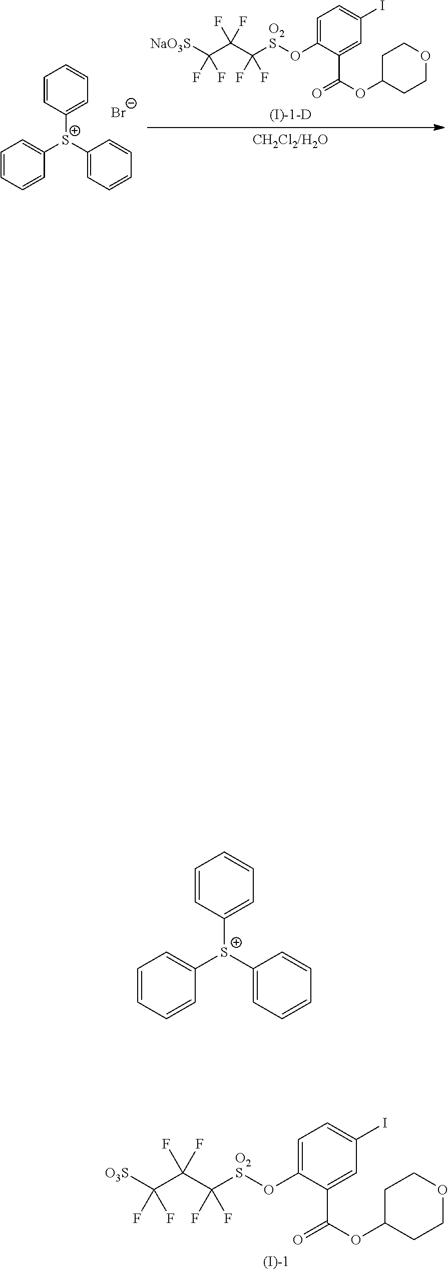

(I)-2 to (I)-22 were synthesized in accordance with the method for synthesizing (I)-1.

### <Resin (A)>

A-1 to A-35 were used as the resin (A) (the resin whose polarity is increased by the action of an acid). A-1 to A-35 used were synthesized in accordance with publicly known methods.

Table 2 below shows the content (mol %) of each repeating unit included in each resin, the weight-average molecular weight (Mw), and the dispersity (Mw/Mn). The content of the repeating unit is the ratio (molar ratio) of each repeating unit relative to all the repeating units included in each resin. Each repeating unit is shown by the structure of the corresponding monomer.

The weight-average molecular weight (Mw) and the dispersity (Mw/Mn) of the resin were (polystyrene-equivalent values) measured by GPC (carrier: tetrahydrofuran (THF)). The contents of the repeating units were measured by 13C-NMR (nuclear magnetic resonance).

TABLE 2

| | Structure of resin | | | | | | | | | |
|---|---|---|---|---|---|---|---|---|---|---|
| Resin | Repeating unit 1 | | Repeating unit 2 | | Repeating unit 3 | | Repeating unit 4 | | | |
| (A) | Type | Content (mol %) | Type | Content (mol %) | Type | Content (mol %) | Type | Content (mol %) | Mw | Mw/Mn |
| A-1 | MB-10 | 50 | MA-16 | 50 | — | — | — | — | 8500 | 1.60 |
| A-2 | MB-15 | 40 | MA-7 | 60 | — | — | — | — | 9000 | 1.70 |
| A-3 | MB-7 | 30 | MB-14 | 10 | MA-6 | 60 | — | — | 7000 | 1.55 |
| A-4 | MB-5 | 50 | MB-12 | 10 | MA-15 | 40 | — | — | 7500 | 1.55 |
| A-5 | MB-3 | 20 | MB-20 | 40 | MA-4 | 40 | — | — | 7000 | 1.60 |
| A-6 | MB-4 | 20 | MB-14 | 30 | MA-2 | 50 | — | — | 6500 | 1.63 |
| A-7 | MB-6 | 30 | MB-19 | 10 | MA-3 | 60 | — | — | 9500 | 1.45 |
| A-8 | MB-9 | 30 | MB-20 | 10 | MA-13 | 60 | — | — | 12000 | 1.65 |
| A-9 | MB-4 | 20 | MB-19 | 20 | MA-2 | 60 | — | — | 6000 | 1.55 |
| A-10 | MB-16 | 30 | MA-17 | 70 | — | — | — | — | 8000 | 1.40 |
| A-11 | MB-4 | 30 | MB-31 | 30 | MA-4 | 40 | — | — | 6500 | 1.65 |
| A-12 | MB-30 | 20 | MA-8 | 80 | — | — | — | — | 5500 | 1.65 |
| A-13 | MB-13 | 50 | MA-10 | 50 | — | — | — | — | 15000 | 1.75 |
| A-14 | MB-8 | 30 | MA-20 | 70 | — | — | — | — | 9000 | 1.60 |
| A-15 | MB-18 | 30 | MB-29 | 30 | MA-14 | 40 | — | — | 8000 | 1.55 |
| A-16 | MB-3 | 30 | MB-20 | 20 | MA-2 | 40 | MA-8 | 10 | 7500 | 1.70 |
| A-17 | MB-1 | 30 | MB-26 | 30 | MA-11 | 40 | — | — | 18000 | 1.80 |
| A-18 | MB-27 | 60 | MA-5 | 40 | — | — | — | — | 7500 | 1.65 |
| A-19 | MB-17 | 30 | MB-24 | 10 | MA-19 | 60 | — | — | 8000 | 1.70 |
| A-20 | MB-11 | 30 | MB-28 | 40 | MA-9 | 30 | — | — | 9500 | 1.80 |
| A-21 | MB-3 | 20 | MB-20 | 10 | MB-32 | 10 | MA-2 | 60 | 10000 | 1.70 |
| A-22 | MB-2 | 60 | MA-1 | 40 | — | — | — | — | 11000 | 1.65 |
| A-23 | MB-21 | 50 | MA-12 | 50 | — | — | — | — | 6500 | 1.60 |
| A-24 | MB-23 | 40 | MA-10 | 60 | — | — | — | — | 8000 | 1.55 |
| A-25 | MB-1 | 20 | MB-19 | 20 | MA-2 | 60 | — | — | 8000 | 1.55 |
| A-26 | MB-25 | 30 | MA-2 | 70 | — | — | — | — | 7500 | 1.60 |
| A-27 | MB-3 | 40 | MA-3 | 60 | — | — | — | — | 9500 | 1.60 |
| A-28 | MB-22 | 40 | MA-4 | 60 | — | — | — | — | 10000 | 1.70 |
| A-29 | MB-3 | 30 | MB-33 | 20 | MA-2 | 50 | — | — | 6600 | 1.63 |
| A-30 | MB-7 | 30 | MB-14 | 10 | MA-18 | 60 | — | — | 8100 | 1.65 |
| A-31 | MB-1 | 20 | MB-19 | 20 | MA-21 | 60 | — | — | 6600 | 1.65 |
| A-32 | MB-17 | 60 | MA-24 | 40 | — | — | — | — | 8200 | 1.71 |
| A-33 | MB-4 | 20 | MB-19 | 20 | MA-23 | 60 | — | — | 6500 | 1.60 |
| A-34 | MB-14 | 30 | MA-22 | 70 | — | — | — | — | 7200 | 1.58 |
| A-35 | MB-19 | 25 | MB-34 | 25 | MA-2 | 50 | — | — | 8400 | 1.59 |

The structural formulae of the monomers (monomers corresponding to the repeating units) described in Table 2 are shown below.

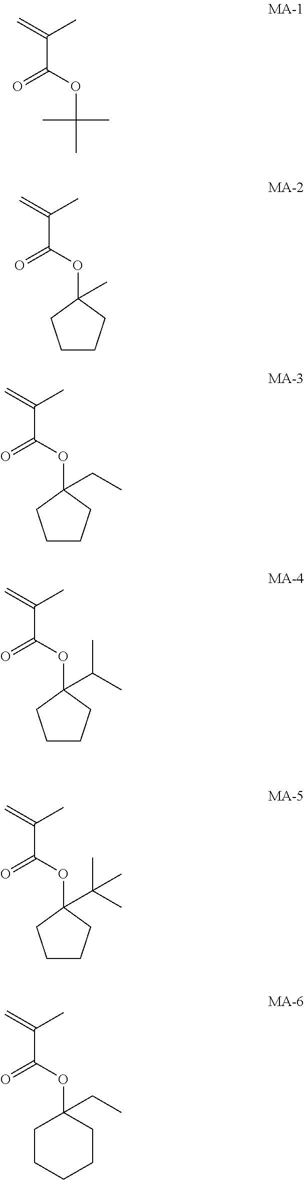

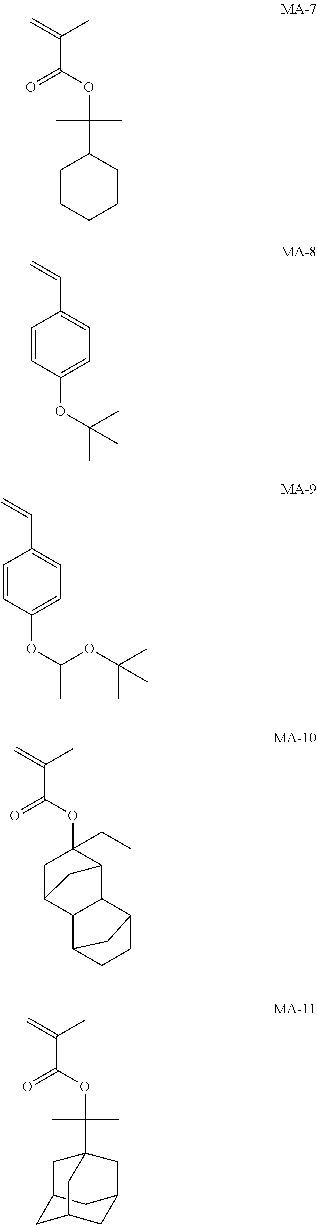

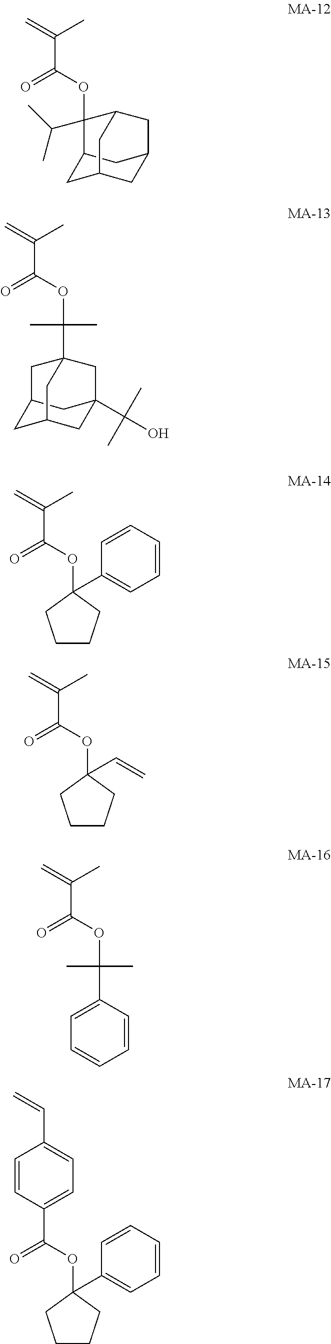

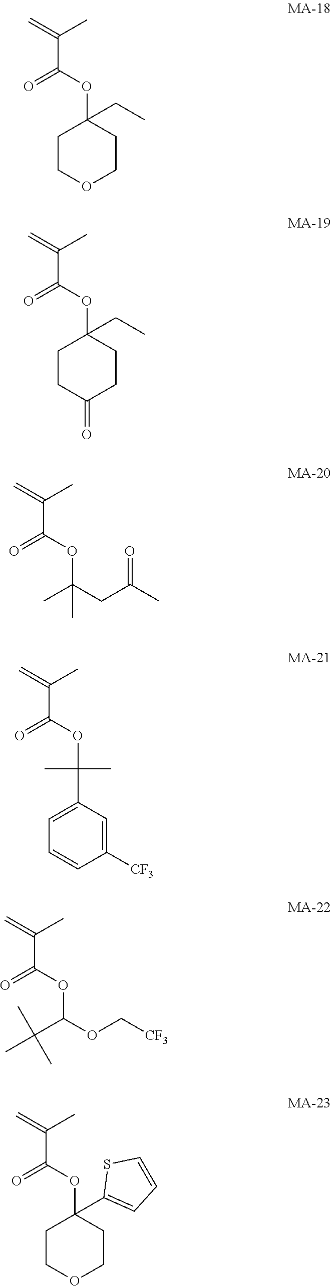

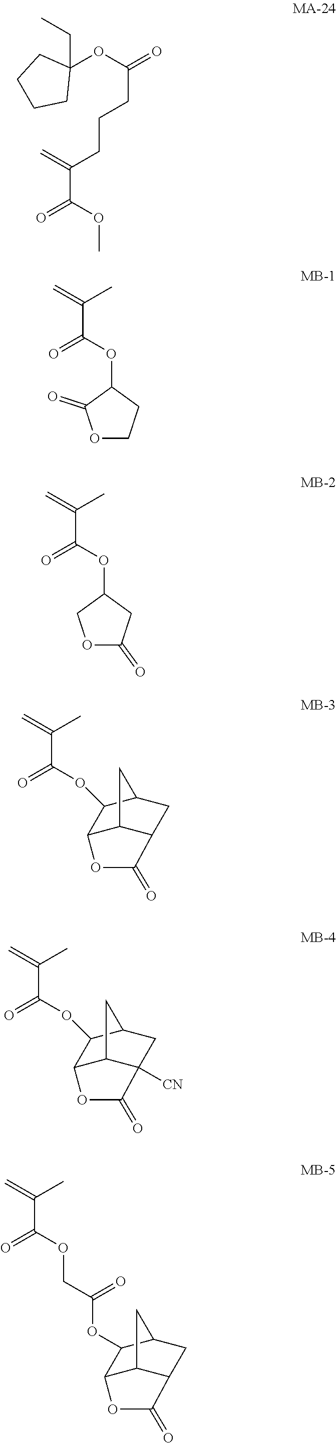

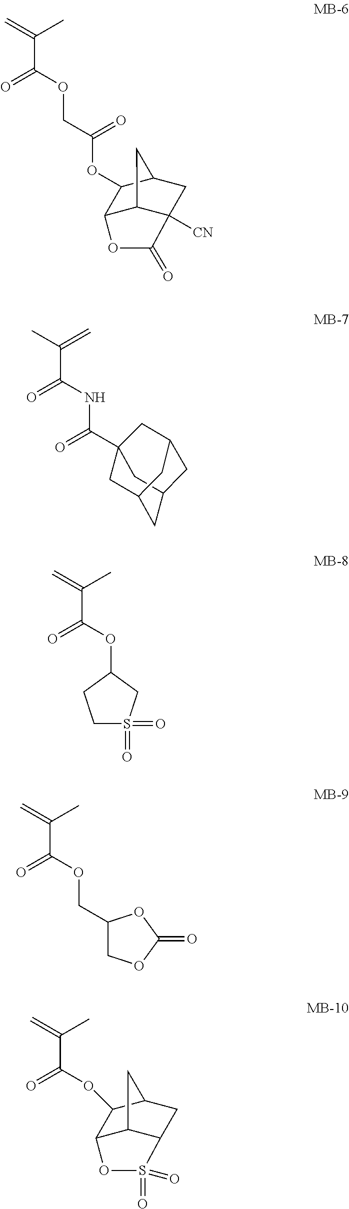

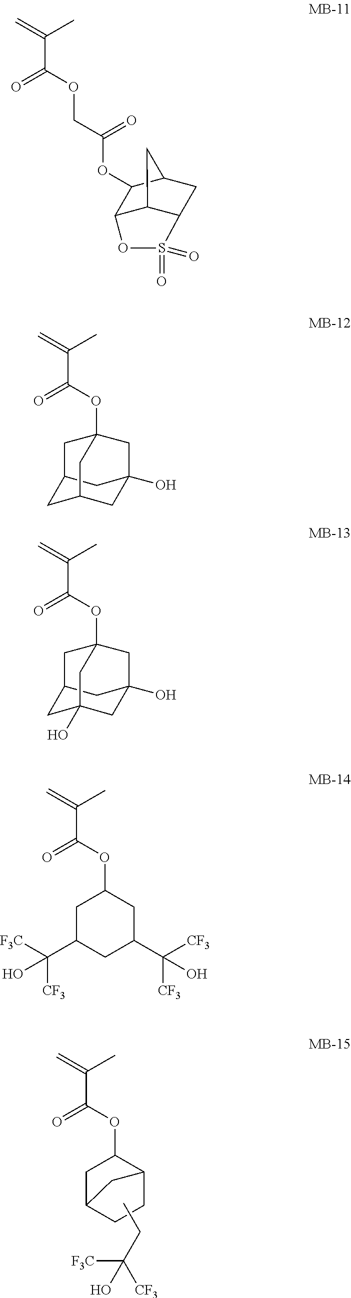

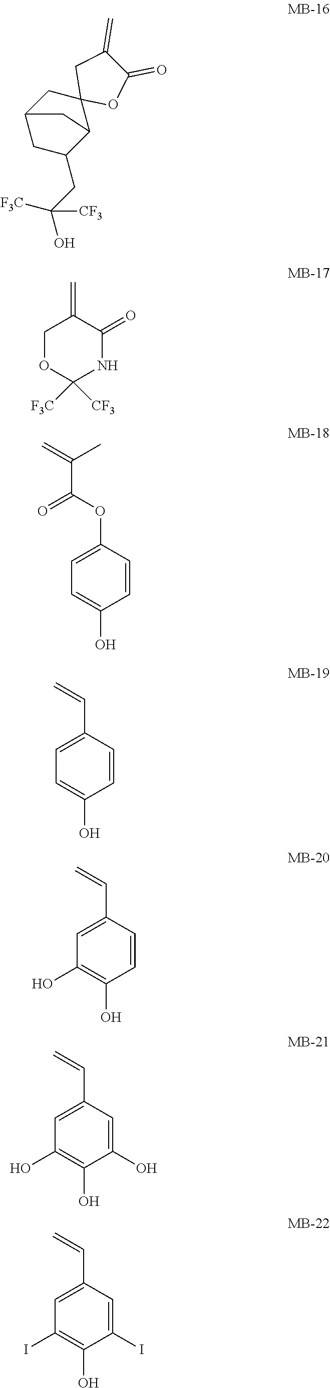

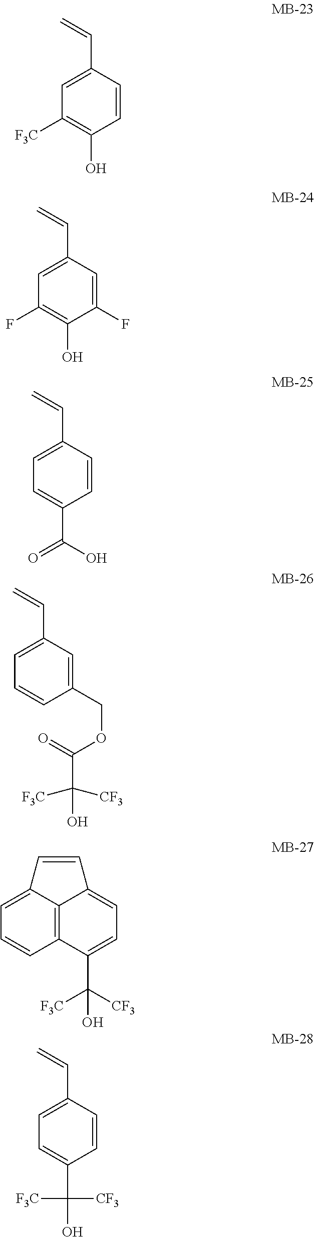

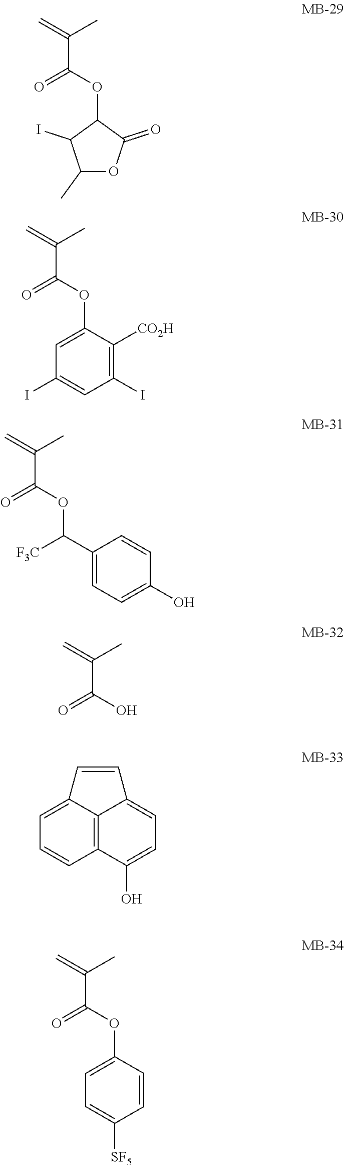

### <Compound (B)>

B-1 to B-20 were used as the compound (B).

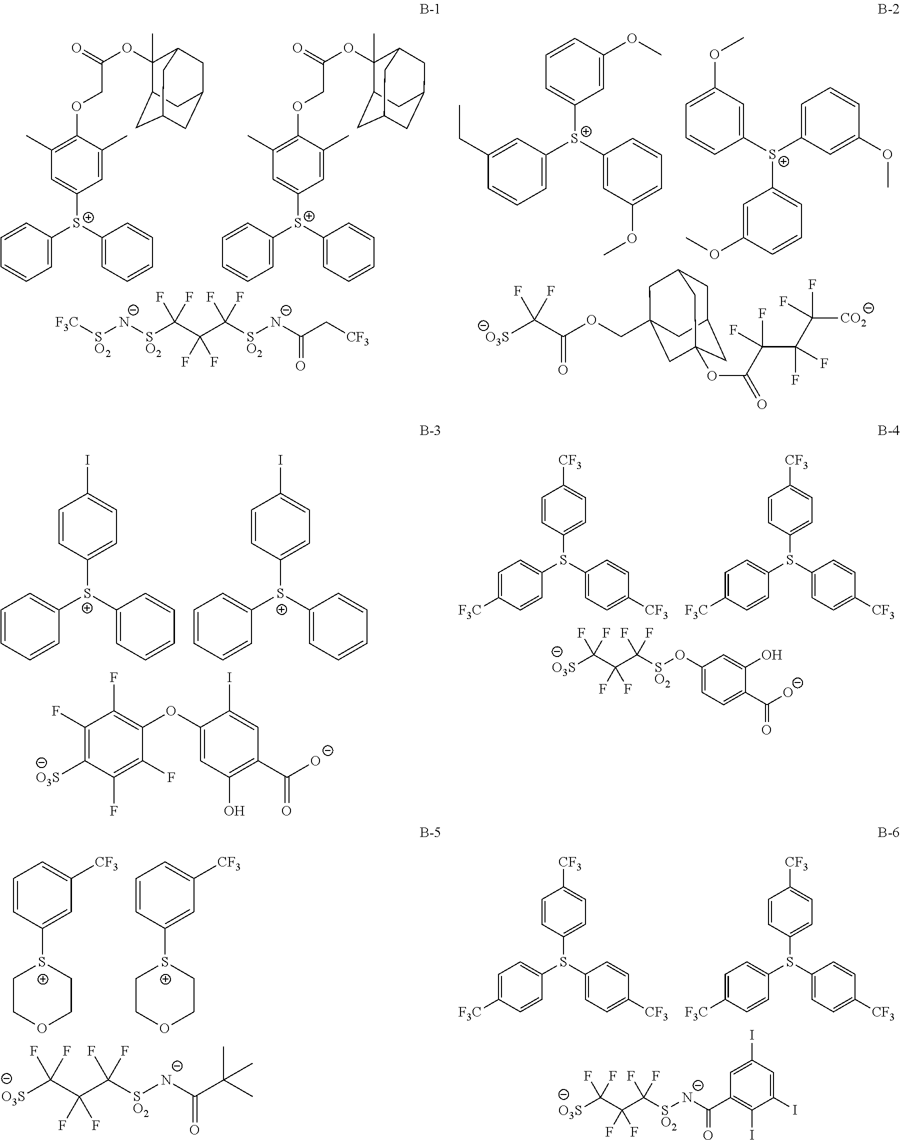

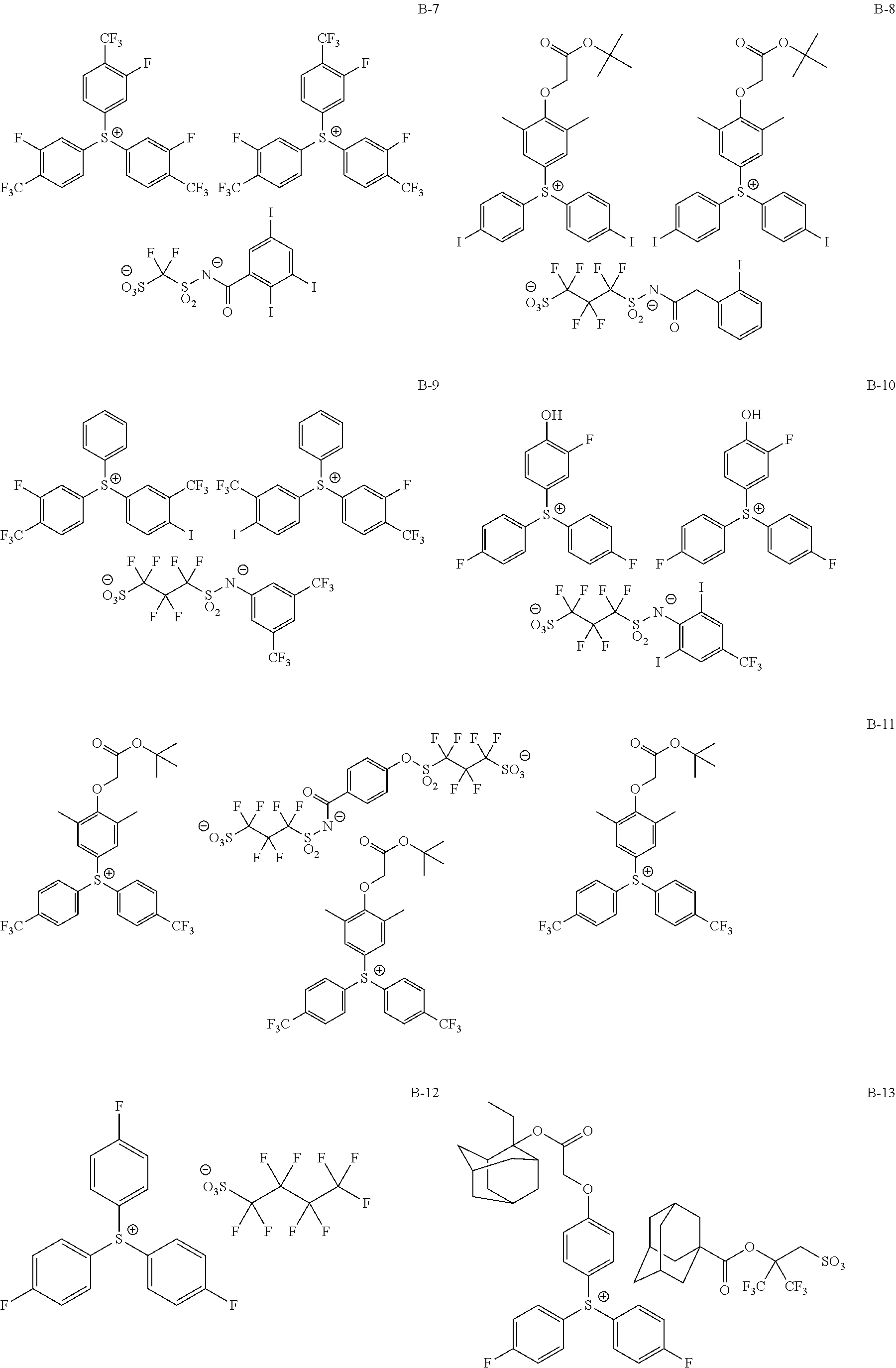

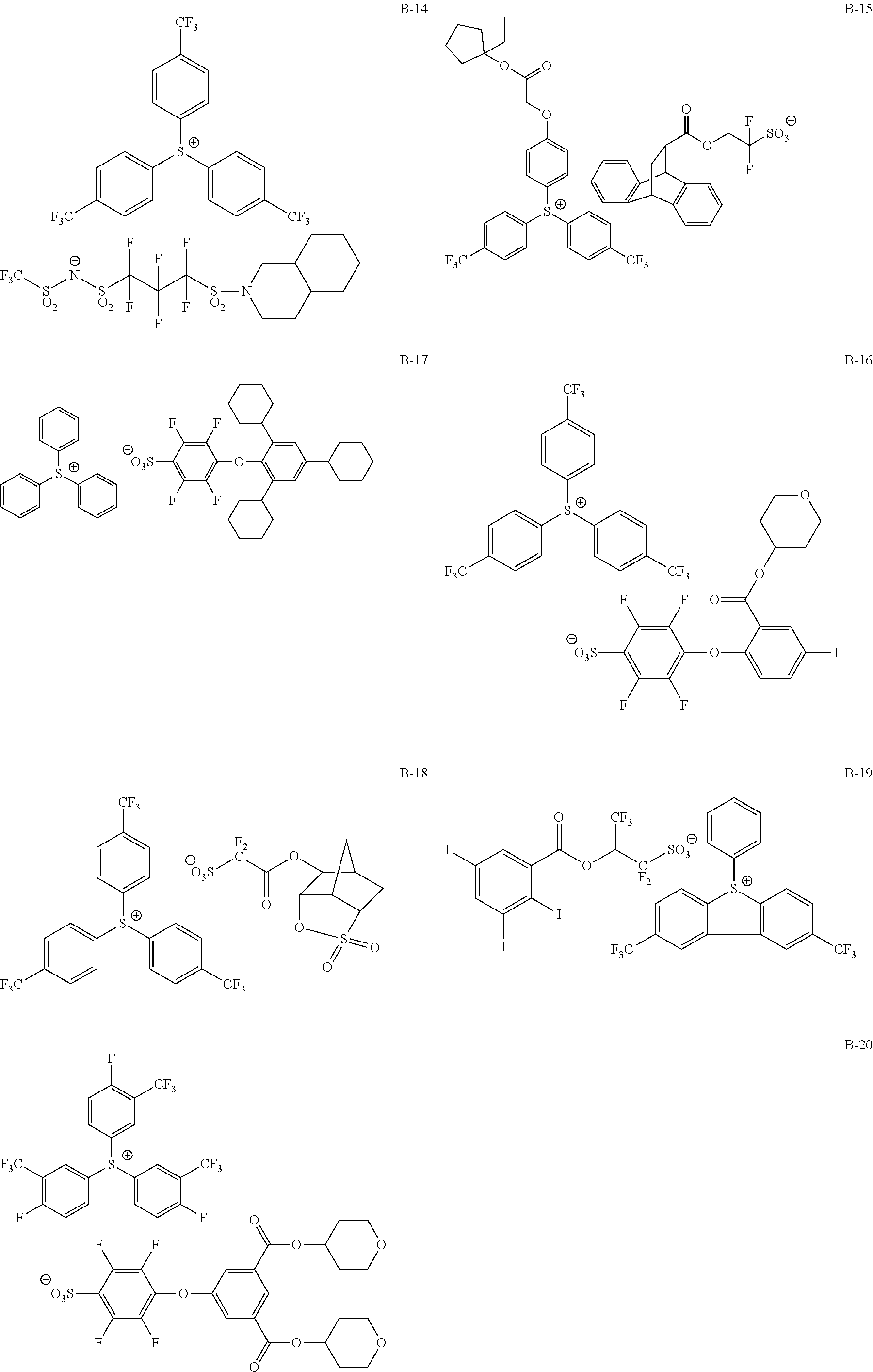

### <Compound (C)>

C-1 to C-14 and D-1 to D-5 were used as the compound (C) (acid diffusion control agent).

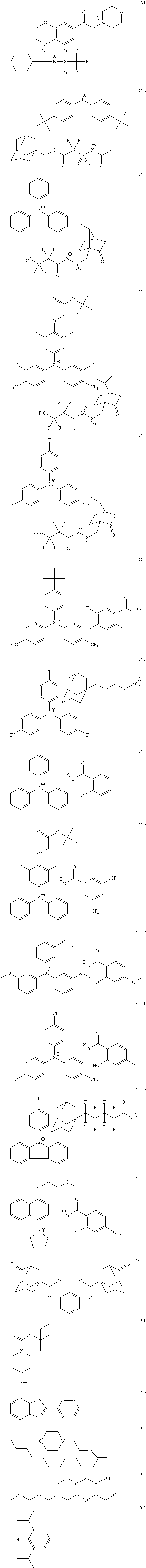

### <Hydrophobic Resin>

E-1 to E-8 were used as the hydrophobic resins.

The structural formulae of repeating units included in E-1 to E-8 are shown below. For each resin, the content (mol %; in order from the left) of each repeating unit based on all the repeating units included in the resin, the weight-average molecular weight (Mw), and the dispersity (Mw/Mn) are shown in Table 3 below.

The content of each repeating unit is the content ratio (molar ratio) of the repeating unit relative to all the repeating units included in the resin.

The weight-average molecular weight (Mw) and the dispersity (Mw/Mn) of the resin were (polystyrene-equivalent values) measured by GPC (carrier: tetrahydrofuran (THF)). The contents of the repeating units were measured by 13C-NMR.

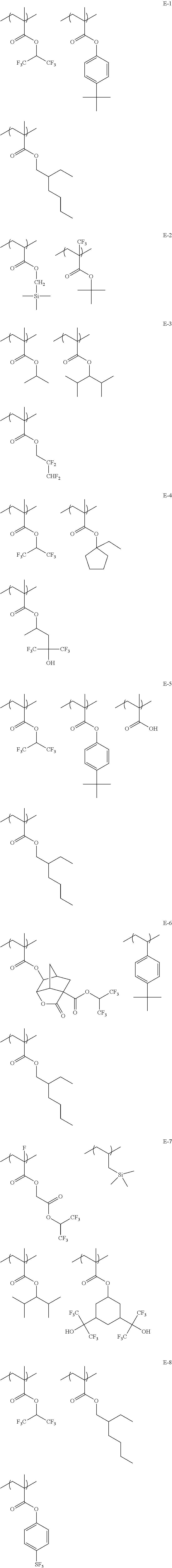

TABLE 3

| Hydrophobic resin | Contents of repeating units (mol %) | | | | Mw | Mw/Mn |
|---|---|---|---|---|---|---|
| E-1 | 50 | 45 | 5 | — | 6500 | 1.52 |
| E-2 | 50 | 50 | — | — | 25000 | 1.65 |
| E-3 | 30 | 65 | 5 | — | 22000 | 1.55 |
| E-4 | 40 | 40 | 20 | — | 12000 | 1.68 |
| E-5 | 40 | 50 | 5 | 5 | 5500 | 1.49 |
| E-6 | 90 | 8 | 2 | — | 12000 | 1.63 |
| E-7 | 20 | 30 | 40 | 10 | 13000 | 1.55 |
| E-8 | 50 | 10 | 40 | — | 9000 | 1.51 |

### <Surfactant>

The surfactants used are as follows.
F-1: MEGAFACE F176 (manufactured by DIC Corporation, fluorine-based surfactant)
F-2: MEGAFACE R08 (manufactured by DIC Corporation, fluorine- and silicon-based surfactant)
F-3: PF656 (manufactured by OMNOVA Solutions, Inc., fluorine-based surfactant)

### <Solvent>

The solvents used are as follows.
G-1: Propylene glycol monomethyl ether acetate (PGMEA)
G-2: Propylene glycol monomethyl ether (PGME)
G-3: Propylene glycol monoethyl ether (PGEE)
G-4: Cyclohexanone
G-5: Cyclopentanone
G-6:2-Heptanone
G-7: Ethyl lactate
G-8: γ-Butyrolactone
G-9: Propylene carbonate
   <Preparation and application of resist composition>

The components shown in Tables 4 and 5 below were mixed such that the concentration of solid contents was 2 mass %. The resulting liquid mixtures were each filtered through a polyethylene filter having a pore size of 0.02 μm to prepare resist compositions.

The “solid contents” mean components other than the solvent.

Such a resist compositions was applied to a 6-inch Si (silicon) wafer that had been treated with hexamethyldisilazane (HMDS) in advance, using a spin coater “Mark8” manufactured by Tokyo Electron Ltd., and dried on a hot plate at 130 °C for 300 seconds to obtain a resist film having a film thickness of 35 nm.

Here, one inch is 0.0254 m.

In Tables 4 and 5, the descriptions separated by “/” in the “Type” columns indicate that a plurality of compounds are included as the corresponding component, and the descriptions separated by “/” in the “mass %” columns show the contents of the plurality of compounds in order.

In Tables 4 and 5, the “mass %” columns each show the content (mass %) of a solid component relative to the total solid contents.

In Tables 4 and 5, the “Mixing ratio” column of “Solvent” shows the mixing ratio (mass ratio) of solvents.

TABLE 4

| | | | | | | | Acid | | | | | | | |
|---|---|---|---|---|---|---|---|---|---|---|---|---|---|---|
| | Compound | | | | Compound | | diffusion | | Hydrophobic | | | | | |
| | (N) | | Resin (A) | | (B) | | control agent | | resin | | Surfactant | | Solvent | |
| Resist | | mass | | mass | | mass | | mass | | mass | | mass | | Mixing |
| composition | Type | % | Type | % | Type | % | Type | % | Type | % | Type | % | Type | ratio |
| Re-1 | (I)-1 | 20.1 | A-25 | 76.3 | — | — | D-1 | 2.2 | E-4 | 1.4 | — | — | G-1/G-6 | 40/60 |
| Re-2 | (I)-2 | 19.5 | A-7 | 73.7 | — | — | C-1 | 4.9 | E-1 | 1.9 | — | — | G-4 | 100 |
| Re-3 | (I)-3 | 18.1 | A-34 | 79.2 | — | — | D-3 | 2.7 | — | — | — | — | G-1 | 100 |
| Re-4 | (I)-4 | 15.6 | A-3 | 77.3 | — | — | C-2 | 6.3 | E-7 | 0.8 | — | — | G-1 | 100 |
| Re-5 | (I)-5 | 28.1 | A-6 | 65.0 | — | — | C-10 | 6.2 | E-2 | 0.7 | — | — | G-1/G-3 | 80/20 |
| Re-6 | (I)-6 | 23.1 | A-20 | 71.8 | — | — | C-6 | 5.0 | — | — | F-1 | 0.1 | G-1/G-9 | 90/10 |
| Re-7 | (I)-7 | 12.1 | A-12 | 82.5 | — | — | C-4 | 4.9 | E-6 | 0.5 | — | — | G-1/G-7 | 80/20 |
| Re-8 | (I)-8 | 20.1 | A-9 | 73.4 | — | — | C-7 | 4.8 | E-3 | 1.7 | — | — | G-1/G-8 | 85/15 |
| Re-9 | (I)-9 | 17.5 | A-15 | 76.0 | — | — | C-3 | 5.2 | E-1 | 1.3 | — | — | G-4 | 100 |
| Re-10 | (I)-10 | 33.4 | A-30 | 61.6 | — | — | D-2 | 3.5 | E-8 | 1.5 | — | — | G-1/G-5 | 50/50 |
| Re-11 | (I)-11 | 16.2 | A-33 | 78.5 | — | — | C-5 | 5.2 | — | — | F-2 | 0.1 | G-1 | 100 |
| Re-12 | (I)-12 | 38.1 | A-12 | 56.8 | — | — | D-4 | 3.2 | E-8 | 1.9 | — | — | G-1/ | 70/25/5 |
| | | | | | | | | | | | | | G-2/G-8 | |
| Re-13 | (I)-13 | 32.9 | A-1 | 60.0 | — | — | C-8 | 4.9 | E-5 | 2.2 | — | — | G-1/G-6 | 40/60 |
| Re-14 | (I)-14 | 25.4 | A-2 | 67.6 | — | — | C-13 | 5.2 | E-2 | 1.8 | — | — | G-1/G-5 | 50/50 |
| Re-15 | (I)-15 | 20.0 | A-27 | 73.3 | — | — | C-12 | 5.1 | E-8 | 1.6 | — | — | G-1 | 100 |
| Re-16 | (I)-16 | 30.1 | A-31 | 58.7 | — | — | C-14 | 8.9 | E-1 | 2.3 | — | — | G-1/ | 70/25/5 |
| | | | | | | | | | | | | | G-2/G-8 | |
| Re-17 | (I)-17 | 39.8 | A-5 | 55.8 | — | — | D-5 | 4.3 | — | — | F-3 | 0.1 | G-7 | 100 |
| Re-18 | (I)-18 | 13.3 | A-23 | 79.5 | — | — | C-9 | 5.5 | E-3 | 1.7 | — | — | G-1/G-8 | 85/15 |
| Re-19 | (I)-19 | 22.0 | A-18 | 69.2 | — | — | C-6 | 6.5 | E-2 | 2.3 | — | — | G-1 | 100 |
| Re-20 | (I)-20 | 19.0 | A-3 | 73.9 | — | — | C-11 | 5.0 | E-3 | 2.1 | — | — | G-7 | 100 |
| Re-21 | (I)-21 | 33.0 | A-16 | 53.9 | — | — | C-5 | 11.2 | E-5 | 1.9 | — | — | G-1 | 100 |
| Re-22 | (I)-22 | 37.0 | A-23 | 45.8 | — | — | C-3 | 15.0 | E-1 | 2.2 | — | — | G-1 | 100 |

TABLE 5

| | Compound | | | | Compound | | | | Hydrophobic | | | | | |
|---|---|---|---|---|---|---|---|---|---|---|---|---|---|---|
| | (N) | | Resin (A) | | (B) | | Acid diffusion | | resin | | Surfactant | | Solvent | |
| Resist | | mass | | mass | | mass | control agent | | | mass | | mass | | Mixing |
| composition | Type | % | Type | % | Type | % | Type | mass % | Type | % | Type | % | Type | ratio |
| Re-23 | (I)-1/ | 6.2/ | A-29 | 81.5 | — | — | C-2 | 4.8 | E-2 | 1.2 | — | — | G-1/ | 70/30 |
| | (I)-4 | 6.3 | | | | | | | | | | | G-2 | |
| Re-24 | (I)-2/ | 13.0/ | A-17 | 59.6 | B-8 | 9.5 | C-5 | 5.6 | E-4 | 2.1 | — | — | G-1/ | 50/50 |
| | (I)-6 | 10.2 | | | | | | | | | | | G-5 | |
| Re-25 | (I)-7/ | 14.1/ | A-10 | 66.1 | B-7 | 10.9 | D-1 | 0.9 | E-2 | 2.2 | — | — | G-1/ | 70/30 |
| | (I)-8 | 5.8 | | | | | | | | | | | G-2 | |
| Re-26 | (I)-3 | 30.9 | A-4 | 61.5 | B-1 | 2.1 | D-2 | 2.8 | E-1 | 2.7 | — | — | G-1/G-2/ | 70/25/5 |
| | | | | | | | | | | | | | G-8 | |
| Re-27 | (I)-13 | 20.1 | A-32 | 59.4 | B-2/ | 12.0/ | C-3 | 4.1 | E-4 | 1.3 | — | — | G-1/ | 40/60 |
| | | | | | B-12 | 3.1 | | | | | | | G-6 | |
| Re-28 | (I)-4 | 24.5 | A-19 | 64.8 | B-3/ | 5.5/ | C-8 | 4.2 | — | — | — | — | G-1/ | 70/30 |
| | | | | | B-18 | 1.0 | | | | | | | G-2 | |
| Re-29 | (I)-5 | 22.1 | A-18 | 57.8 | B-4 | 15.1 | C-4 | 3.3 | E-2 | 1.7 | — | — | G-1/ | 80/20 |
| | | | | | | | | | | | | | G-7 | |
| Re-30 | (I)-7 | 24.7 | A-8 | 65.2 | B-5/ | 5.3/ | D-5 | 2.5 | — | — | — | — | G-1 | 100 |
| | | | | | B-13 | 2.3 | | | | | | | | |
| Re-31 | (I)-8 | 45.3 | A-14 | 48.5 | B-6 | 1.1 | D-4 | 5.1 | — | — | — | — | G-1/ | 85/15 |
| | | | | | | | | | | | | | G-8 | |
| Re-32 | (I)-2 | 23.8 | A-22/ | 24.6/ | B-7 | 9.6 | C-14 | 3.8 | E-3 | 1.4 | F-1 | 0.1 | G-1 | 100 |
| | | | A-24 | 36.7 | | | | | | | | | | |
| Re-33 | (I)-3 | 20.9 | A-11 | 63.8 | B-8/ | 5.3/ | D-3 | 3.8 | E-3 | 1.9 | — | — | G-4 | 100 |
| | | | | | B-19 | 4.3 | | | | | | | | |
| Re-34 | (I)-11 | 25.6 | A-16 | 60.6 | B-9 | 12.7 | D-2 | 1.1 | — | — | — | — | G-1/ | 40/60 |
| | | | | | | | | | | | | | G-6 | |
| Re-35 | (I)-5/ | 8.7/ | A-26 | 58.8 | B-10/ | 12.5/ | C-13 | 2.8 | E-4 | 1.1 | — | — | G-4 | 100 |
| | (I)-8 | 12.7 | | | B-14 | 3.4 | | | | | | | | |
| Re-36 | (I)-4 | 14.2 | A-13 | 62.2 | B-11/ | 15.8/ | C-11 | 4.5 | E-7 | 0.9 | — | — | G-4 | 100 |
| | | | | | B-15 | 2.4 | | | | | | | | |
| Re-37 | (I)-9 | 19.2 | A-21 | 56.5 | B-5/ | 15.3/ | D-3 | 4.3 | E-7 | 1.8 | F-2 | 0.1 | G-1 | 100 |
| | | | | | B-16 | 2.8 | | | | | | | | |
| Re-38 | (I)-12 | 20.1 | A-28 | 64.4 | B-3/ | 4.2/ | D-1 | 3.2 | E-8 | 1.9 | — | — | G-1/ | 85/15 |
| | | | | | B-17 | 6.2 | | | | | | | G-8 | |
| Re-39 | (I)-15 | 15.6 | A-1 | 66.8 | B-1/ | 15.9/ | C-10 | 0.5 | E-1 | 0.7 | F-1 | 0.1 | G-7 | 100 |
| | | | | | B-20 | 0.4 | | | | | | | | |
| Re-40 | (I)-1 | 31.2 | A-7 | 58.4 | B-4/ | 1.3/ | C-3/ | 1.7/ | E-4 | 2.1 | — | — | G-1/ | 50/50 |
| | | | | | B-6 | 1.4 | C-7/ | 1.4/ | | | | | G-5 | |
| | | | | | | | D-2 | 2.5 | | | | | | |
| Re-41 | (I)-2 | 19.8 | A-9 | 63.3 | B-7 | 12.5 | C-9/ | 0.9/ | E-8 | 1.6 | — | — | G-1/ | 70/30 |
| | | | | | | | C-14 | 1.9 | | | | | G-2 | |
| Re-42 | (I)-18 | 36.5 | A-25 | 60.2 | — | — | D-2/ | 1.4/ | E-5 | 0.8 | — | — | G-1 | 100 |
| | | | | | | | D-4 | 1.1 | | | | | | |
| Re-43 | (I)-5 | 22.3 | A-35 | 64.2 | B-4 | 9.1 | D-3 | 1.9 | E-6 | 2.3 | F-1/ | 0.1/ | G-1/ | 80/20 |
| | | | | | | | | | | | F-2 | 0.1 | G-3 | |
| Re-C1 | (Z)-1 | 21.5 | A-9 | 57.7 | B-3 | 15.6 | C-3 | 3.1 | E-3 | 2.1 | — | — | G-1/ | 40/60 |
| | | | | | | | | | | | | | G-6 | |

### <Exposure and Development>

### [EUV Exposure]

The wafer coated with the resist film obtained above was subjected to pattern exposure using an EUV exposure apparatus manufactured by Exitech Ltd. (Micro Exposure Tool, NA (numerical aperture): 0.3, Quadrupole, outer sigma: 0.68, inner sigma: 0.36). An exposure mask having a 1:1 line-and-space pattern with a line width of 20 nm was used.

### [Organic Solvent Development]

The exposed wafer was heated on a hot plate at 90 °C for 60 seconds, then developed with n-butyl acetate for 30 seconds, and spin-dried to obtain a negative resist pattern.

Through the above procedure, resist patterns of Examples 1-1 to 1-43 and Comparative Example 1-1 described in Table 6 below were obtained.

### [Alkali Development]

The exposed wafer was heated on a hot plate at 100 °C for 90 seconds, then immersed in a 2.38 mass % aqueous tetramethylammonium hydroxide (TMAH) solution for 60 seconds, and then rinsed with water for 30 seconds. Subsequently, the wafer was rotated at a rotational speed of 4,000 rpm for 30 seconds, and then dried by baking at 95 °C for 60 seconds to obtain a positive resist pattern.

Through the above procedure, resist patterns of Examples 2-1 to 2-43 and Comparative Example 2-1 described in Table 7 below were obtained.

### <Sensitivity Evaluation (Part 1)>

The resist patterns of Examples and Comparative Example (Examples 1-1 to 1-43 and Comparative Example 1-1) obtained by the organic solvent development were evaluated according to the following procedure.

A cross-sectional shape of each of the obtained patterns of Examples and Comparative Example was observed with a scanning electron microscope (S-9380II manufactured by Hitachi, Ltd.), and an optimum exposure dose when a 1:1 line-and-space resist pattern with a line width of 20 nm was resolved was defined as the sensitivity (Eopt) (mJ/cm²). The evaluation was performed according to the following evaluation criteria.

### [Evaluation Criteria]

A: (Eopt)≤45
B: 45<(Eopt)≤50
C: 50<(Eopt)
   <Pattern shape evaluation (Part 1)>

The resist patterns of Examples and Comparative Example (Examples 1-1 to 1-43 and Comparative Example 1-1) obtained by the organic solvent development were evaluated according to the following procedure.

For each of the Examples and Comparative Example, a cross-sectional shape of the obtained line pattern with an average line width of 20 nm was observed with a critical dimension scanning electron microscope (SEM, S-9380II manufactured by Hitachi, Ltd.), and a pattern line width Lb in a bottom portion of the resist pattern and a pattern line width La in an upper portion of the resist pattern were measured. The value of La/Lb was used as an indicator, and the cross-sectional rectangularity of the pattern shape was evaluated according to the following criteria. S is the best, and E is the worst. Practically, a rating of D or higher is desirable.

### [Evaluation Criteria]

S: 1.00≤(La/Lb)≤1.01
A: 1.01<(La/Lb)≤1.02
B: 1.02<(La/Lb)≤1.03
C: 1.03<(La/Lb)≤1.04
D: 1.04<(La/Lb)≤1.05
E: 1.05<(La/Lb)

### <Sensitivity Evaluation (Part 2)>

The resist patterns of Examples and Comparative Example (Examples 2-1 to 2-43 and Comparative Example 2-1) obtained by the alkaline aqueous solution development were evaluated according to the following procedure.

A cross-sectional shape of each of the obtained patterns of Examples and Comparative Example was observed with a scanning electron microscope (S-9380II manufactured by Hitachi, Ltd.), and an optimum exposure dose when a 1:1 line-and-space resist pattern with a line width of 20 nm was resolved was defined as the sensitivity (Eopt) (mJ/cm²).

### [Evaluation Criteria]

A: (Eopt)≤45
B: 45<(Eopt)≤50
C: 50<(Eopt)

### <Pattern Shape Evaluation (Part 2)>

The resist patterns of Examples and Comparative Example (Examples 2-1 to 2-43 and Comparative Example 2-1) obtained by the alkali development were evaluated according to the following procedure.

For each of the Examples and Comparative Example, a cross-sectional shape of the obtained line pattern with an average line width of 20 nm was observed with a critical dimension scanning electron microscope (SEM, S-9380II manufactured by Hitachi, Ltd.), and a pattern line width Lb in a bottom portion of the resist pattern and a pattern line width La in an upper portion of the resist pattern were measured. The value of Lb/La was used as an indicator, and the cross-sectional rectangularity of the pattern shape was evaluated according to the following criteria. S is the best, and E is the worst. Practically, a rating of D or higher is desirable.

### [Evaluation Criteria]

S: 1.00≤(Lb/La)≤1.01
A: 1.01<(Lb/La)≤1.02
B: 1.02<(Lb/La)≤1.03
C: 1.03<(Lb/La)≤1.04
D: 1.04<(Lb/La)≤1.05
E: 1.05<(Lb/La)

TABLE 6

| | Resist composition | Sensitivity | Pattern shape |
|---|---|---|---|
| Example 1-1 | Re-1 | A | S |
| Example 1-2 | Re-2 | A | B |
| Example 1-3 | Re-3 | A | B |
| Example 1-4 | Re-4 | A | S |
| Example 1-5 | Re-5 | A | S |
| Example 1-6 | Re-6 | B | C |
| Example 1-7 | Re-7 | A | S |
| Example 1-8 | Re-8 | A | S |
| Example 1-9 | Re-9 | A | S |
| Example 1-10 | Re-10 | A | D |
| Example 1-11 | Re-11 | A | S |
| Example 1-12 | Re-12 | A | A |
| Example 1-13 | Re-13 | A | A |
| Example 1-14 | Re-14 | A | S |
| Example 1-15 | Re-15 | A | A |
| Example 1-16 | Re-16 | A | S |
| Example 1-17 | Re-17 | A | B |
| Example 1-18 | Re-18 | A | A |
| Example 1-19 | Re-19 | A | A |
| Example 1-20 | Re-20 | A | S |
| Example 1-21 | Re-21 | A | B |
| Example 1-22 | Re-22 | A | A |
| Example 1-23 | Re-23 | A | A |
| Example 1-24 | Re-24 | A | B |
| Example 1-25 | Re-25 | A | S |
| Example 1-26 | Re-26 | A | S |
| Example 1-27 | Re-27 | A | A |
| Example 1-28 | Re-28 | A | S |
| Example 1-29 | Re-29 | A | S |
| Example 1-30 | Re-30 | A | S |
| Example 1-31 | Re-31 | A | S |
| Example 1-32 | Re-32 | A | B |
| Example 1-33 | Re-33 | A | S |
| Example 1-34 | Re-34 | A | S |
| Example 1-35 | Re-35 | A | S |
| Example 1-36 | Re-36 | A | S |
| Example 1-37 | Re-37 | A | S |
| Example 1-38 | Re-38 | A | A |
| Example 1-39 | Re-39 | A | A |
| Example 1-40 | Re-40 | A | B |
| Example 1-41 | Re-41 | A | B |
| Example 1-42 | Re-42 | A | A |
| Example 1-43 | Re-43 | A | S |
| Comparative | Re-C1 | C | E |
| Example 1-1 | | | |

TABLE 7

| | Resist composition | Sensitivity | Pattern shape |
|---|---|---|---|
| Example 2-1 | Re-1 | A | S |
| Example 2-2 | Re-2 | A | B |
| Example 2-3 | Re-3 | A | B |
| Example 2-4 | Re-4 | A | S |
| Example 2-5 | Re-5 | A | S |
| Example 2-6 | Re-6 | B | C |
| Example 2-7 | Re-7 | A | S |
| Example 2-8 | Re-8 | A | S |
| Example 2-9 | Re-9 | A | S |
| Example 2-10 | Re-10 | A | D |
| Example 2-11 | Re-11 | A | S |
| Example 2-12 | Re-12 | A | A |
| Example 2-13 | Re-13 | A | A |
| Example 2-14 | Re-14 | A | S |
| Example 2-15 | Re-15 | A | A |
| Example 2-16 | Re-16 | A | S |
| Example 2-17 | Re-17 | A | B |
| Example 2-18 | Re-18 | A | A |
| Example 2-19 | Re-19 | A | A |
| Example 2-20 | Re-20 | A | S |
| Example 2-21 | Re-21 | A | B |
| Example 2-22 | Re-22 | A | A |
| Example 2-23 | Re-23 | A | A |
| Example 2-24 | Re-24 | A | B |
| Example 2-25 | Re-25 | A | S |
| Example 2-26 | Re-26 | A | S |
| Example 2-27 | Re-27 | A | A |
| Example 2-28 | Re-28 | A | S |
| Example 2-29 | Re-29 | A | S |
| Example 2-30 | Re-30 | A | S |
| Example 2-31 | Re-31 | A | S |
| Example 2-32 | Re-32 | A | B |
| Example 2-33 | Re-33 | A | S |
| Example 2-34 | Re-34 | A | S |
| Example 2-35 | Re-35 | A | S |
| Example 2-36 | Re-36 | A | S |
| Example 2-37 | Re-37 | A | S |
| Example 2-38 | Re-38 | A | A |
| Example 2-39 | Re-39 | A | A |
| Example 2-40 | Re-40 | A | B |
| Example 2-41 | Re-41 | A | B |
| Example 2-42 | Re-42 | A | A |
| Example 2-43 | Re-43 | A | S |
| Comparative | Re-C1 | C | E |
| Example 2-1 | | | |

The results shown in Tables 6 and 7 showed that the resist compositions used in Examples each had high sensitivity and provided a pattern having a good shape.

The present invention can provide an actinic ray-sensitive or radiation-sensitive resin composition that has high sensitivity and that provides a pattern having a good shape.

Furthermore, the present invention can provide a resist film formed using the actinic ray-sensitive or radiation-sensitive resin composition, and a pattern forming method and a method for producing an electronic device that use the actinic ray-sensitive or radiation-sensitive resin composition.

While the present invention has been described in detail and with reference to specific embodiments, it will be apparent to those skilled in the art that various changes and modifications can be made without departing from the spirit and scope of the present invention.

What is claimed is:

1. An actinic ray-sensitive or radiation-sensitive resin composition comprising:
a compound (N) represented by a formula (a-1) below; and
a resin, 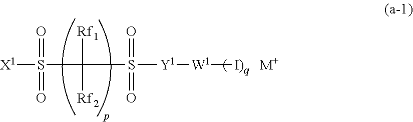
wherein in the formula (a-1),
X¹ represents —O⁻ or —N⁻—SO₂—Rf₃,
Rf₁, Rf₂, and Rf₃ each independently represent a fluorine atom or an alkyl group having at least one fluorine atom, when a plurality of Rf₁'s and a plurality of Rf₂'s are present, the plurality of Rf₁'s may be the same or different and the plurality of Rf₂'s may be the same or different,
Y¹ represents a single bond, —O—, or —S—,
W¹ represents an organic group,
p represents an integer of 1 or more,
q represents an integer of 1 or more, and
M⁺ represents a cation.

2. The actinic ray-sensitive or radiation-sensitive resin composition according to claim 1, wherein the compound (N) is represented by a formula (a-2) below: 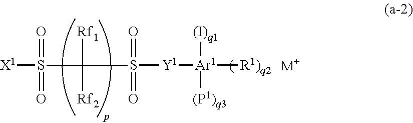
in the formula (a-2),
X¹ represents —O⁻ or —N⁻—SO₂—Rf₃,
Rf₁, Rf₂, and Rf₃ each independently represent a fluorine atom or an alkyl group having at least one fluorine atom, when a plurality of Rf₁'s and a plurality of Rf₂'s are present, the plurality of Rf₁'s may be the same or different and the plurality of Rf₂'s may be the same or different,
Y¹ represents a single bond, —O—, or —S—,
Ar¹ represents an aromatic group,
R¹ represents an organic group having an iodine atom,
p represents an integer of 1 or more,
q1 and q2 each independently represent an integer of 0 or more, provided that at least one of q1 or q2 represents an integer of 1 or more,
P¹ represents an organic group having no iodine atom,
q3 represents an integer of 0 or more, and
M⁺ represents a cation.

3. The actinic ray-sensitive or radiation-sensitive resin composition according to claim 1, wherein X¹ in the formula (a-1) represents —O⁻.

4. The actinic ray-sensitive or radiation-sensitive resin composition according to claim 2, wherein q1 in the formula (a-2) represents an integer of 1 or more.

5. The actinic ray-sensitive or radiation-sensitive resin composition according to claim 2, wherein q2 in the formula (a-2) represents an integer of 1 or more.

6. The actinic ray-sensitive or radiation-sensitive resin composition according to claim 1, wherein p in the formula (a-1) represents an integer of 1 to 3.

7. The actinic ray-sensitive or radiation-sensitive resin composition according to claim 5, wherein at least one R¹ in the formula (a-2) represents —CO—O—R², —O—CO—R², —O—CO—O—R², —SO₂—R², or —SO₃—R², where R² represents an organic group having an iodine atom.

8. The actinic ray-sensitive or radiation-sensitive resin composition according to claim 2, wherein q3 in the formula (a-2) represents an integer of 1 or more, and at least one P¹ represents —CO—O—P², —O—CO—P², —O—CO—O—P², —SO₂—P², or —SO₃—P², where P² represents an organic group having no iodine atom.

9. The actinic ray-sensitive or radiation-sensitive resin composition according to claim 1, wherein M⁺ in the formula (a-1) represents a sulfonium cation or an iodonium cation.

10. The actinic ray-sensitive or radiation-sensitive resin composition according to claim 1, wherein q in the formula (a-1) represents an integer of 1 to 20.

11. The actinic ray-sensitive or radiation-sensitive resin composition according to claim 2, wherein R¹ in the formula (a-2) has no aromatic ring.

12. A resist film formed using the actinic ray-sensitive or radiation-sensitive resin composition according to claim 1.

13. A pattern forming method comprising:
forming a resist film on a substrate using the actinic ray-sensitive or radiation-sensitive resin composition according to claim 1;
exposing the resist film; and
developing the exposed resist film using a developer.

14. A method for producing an electronic device, the method comprising the pattern forming method according to claim 13.
